SEQUENCE LISTING (226 sequences)

SEQ ID NO: 1 - protein, 159 aa, Homo sapiens
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 2 - protein, 137 aa, Homo sapiens
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 3 - protein, 137 aa, Artificial Sequence
  Description of Artificial Sequence Synthetic polypeptide
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 4 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 5 - protein, 77 aa, Homo sapiens
ITCPPPMSVEHADIWVKSYSLYSRERYICNSGFKRKAGTSSLTECVLNKATNVAHWTTPSLKCIRDPALVHQRPAPP

SEQ ID NO: 6 - protein, 361 aa, Artificial Sequence
  Description of Artificial Sequence Synthetic polypeptide
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 7 - protein, 361 aa, Artificial Sequence
  Description of Artificial Sequence Synthetic polypeptide
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSCPPPMSVEHADIWVKSYSLYSRERYICNSGFKRKAGTSSLTECVLNKATNVAHWTTPSLKCIRDPALVHQRPAPPSGGSGGGGSGGGSGGGGSLQDWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 8 - protein, 361 aa, Artificial Sequence
  Description of Artificial Sequence Synthetic polypeptide
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVDVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 9 - protein, 361 aa, Artificial Sequence
  Description of Artificial Sequence Synthetic polypeptide
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISNLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 10 - protein, 361 aa, Artificial Sequence
  Description of Artificial Sequence Synthetic polypeptide
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESNVHPSCKVTAMKCFLLELQVISLESGDASIHDTVENLIILANNSLSSNGNVTESGCKECEELEEKNIKEFLQSFVHIVQMFINTS

SEQ ID NO: 11 - protein, 361 aa, Artificial Sequence
  Description of Artificial Sequence Synthetic polypeptide
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHNTVENLIILANNSLSSNGNVTESGCKECEELEEKNIKEFLQSFVHIVQMFINTS

SEQ ID NO: 12 - protein, 361 aa, Artificial Sequence
  Description of Artificial Sequence Synthetic polypeptide
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVQNLIILANNSLSSNGNVTESGCKECEELEEKNIKEFLQSFVHIVQMFINTS

SEQ ID NO: 13 - protein, 361 aa, Artificial Sequence
  Description of Artificial Sequence Synthetic polypeptide
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTS

SEQ ID NO: 14 - protein, 361 aa, Artificial Sequence
  Description of Artificial Sequence Synthetic polypeptide
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 15 - protein, 361 aa, Artificial Sequence
  Description of Artificial Sequence Synthetic polypeptide
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVEMFINTS

SEQ ID NO: 16 - protein, 361 aa, Artificial Sequence
  Description of Artificial Sequence Synthetic polypeptide
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVDVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTS

SEQ ID NO: 17 - protein, 361 aa, Artificial Sequence
  Description of Artificial Sequence Synthetic polypeptide
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSCPPPMSVEHADIWVKSYSLYSRERYICNSGFKRKAGTSSLTECVLNKATNVAHWTTPSLKCIRDPALVHQRPAPPSGGSGGGGSGGGSGGGGSLQDWVNVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTS

SEQ ID NO: 18 - protein, 361 aa, Artificial Sequence
  Description of Artificial Sequence Synthetic polypeptide
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESNVHPSCKVTAMKCFLLELQVISLESGDASIHDTVQDLIILANNSLSSNGNVTESGCKECEELEEKNIKEFLQSFVHIVQMFINTS

SEQ ID NO: 19 - protein, 625 aa, Artificial Sequence
  Description of Artificial Sequence Synthetic polypeptide
EVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSSGGGGSGGGGSGGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 20 - protein, 369 aa, Artificial Sequence
  Description of Artificial Sequence Synthetic polypeptide
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPP

SEQ ID NO: 21 - protein, 369 aa, Artificial Sequence
  Description of Artificial Sequence Synthetic polypeptide
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 22 - protein, 363 aa, Artificial Sequence
  Description of Artificial Sequence Synthetic polypeptide
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 23 - protein, 374 aa, Artificial Sequence
  Description of Artificial Sequence Synthetic polypeptide
ITCPPPMSVEHADIWVKSYSLYSRERYICNSGFKRKAGTSSLTECVLNKATNVAHWTTPSLKCIRDPALVHQRPAPPSGGGSGGGGSGGGGSGGGGSGGGSLQQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 24 - protein, 362 aa, Artificial Sequence
  Description of Artificial Sequence Synthetic polypeptide
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGSGGGGSGGGSGGGGSLQCPPPMSVEHADIWVKSYSLYSRERYICNSGFKRKAGTSSLTECVLNKATNVAHWTTPSLKCIRDPALVHQRPAPPSG

SEQ ID NO: 25 - protein, 266 aa, Artificial Sequence
  Description of Artificial Sequence Synthetic polypeptide
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSGGGGSGGGGSQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 26 - protein, 266 aa, Artificial Sequence
  Description of Artificial Sequence Synthetic polypeptide
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 27 - protein, 809 aa, Artificial Sequence
  Description of Artificial Sequence Synthetic polypeptide
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFKQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 28 - protein, 817 aa, Artificial Sequence
  Description of Artificial Sequence Synthetic polypeptide
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFKQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 29 - protein, 646 aa, Artificial Sequence
  Description of Artificial Sequence Synthetic polypeptide
EVQLLESGGGLVQPGGSLRLSCAASGFTFSAYRMFWVRQAPGKGLEWVSSIYPSGGITFYADSVKGRFTISRDNSKNTLYLQMNSLRAEDTAIYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSCPPPMSVEHADIWVKSYSLYSRERYICNSGFKRKAGTSSLTECVLNKATNVAHWTTPSLKCIRDSGGSGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 30 - protein, 215 aa, Artificial Sequence
  Description of Artificial Sequence Synthetic polypeptide
QSALTQPASVSGSPGQSITISCTGTSSDVGAYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTEC

SEQ ID NO: 31 - protein, 133 aa, Artificial Sequence
  Description of Artificial Sequence Synthetic polypeptide
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 32 - protein, 285 aa, Artificial Sequence
  Description of Artificial Sequence Synthetic polypeptide
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 33 - protein, 117 aa, Artificial Sequence
  Description of Artificial Sequence Synthetic polypeptide
QVQLQESGPGLVKPSETLSLTCTVSGFSLTSYDISWIRQPPGKGLEWIGVIWTGVGTNYNPSLKSRVTISVDTSKNQFSLKLSSVTAADTAVYYCARDPYYYGMDYWGQGTLVTVSS

SEQ ID NO: 34 - protein, 107 aa, Artificial Sequence
  Description of Artificial Sequence Synthetic polypeptide
DIQMTQSPSSLSASVGDRVTITCRASQDISIWLSWYQQKPGKAPKLLIYKASNLHTGVPSRFSGSGSGTDFTLTISSLQPEDFATYYCLQSQSFPRTFGQGTKLEIK

SEQ ID NO: 35 - protein, 124 aa, Artificial Sequence
  Description of Artificial Sequence Synthetic polypeptide
QVQLVESGGGVVQPGRSLRLSCAASGFTFSSYGMHWVRQAPGKGLEWVAVIWYEGSNKYYADSVKGRFTISRDNSKNTLYLQMNSLRAEDTAVYYCARGGSMVRGDYYYGMDVWGQGTTVTVSS

SEQ ID NO: 36 - protein, 107 aa, Artificial Sequence
  Description of Artificial Sequence Synthetic polypeptide
AIQLTQSPSSLSASVGDRVTITCRASQGISSALAWYQQKPGKAPKLLIYDASSLESGVPSRFSGSGSGTDFTLTISSLQPEDFATYYCQQFNSYPYTFGQGTKLEIK

SEQ ID NO: 37 - protein, 123 aa, Artificial Sequence
  Description of Artificial Sequence Synthetic polypeptide
QVQLVESGGGVVQPGRSLRLSCAASGFTFSSYGMHWVRQAPGKGLEWMAVIWYVGSNKYYADSVKGRFTISRDNSKNTLYLQMNSLRAEDTAVYYCARGGELGRDYYSGMDVWGQGTTVTVSS

SEQ ID NO: 38 - protein, 108 aa, Artificial Sequence
  Description of Artificial Sequence Synthetic polypeptide
DIQMTQSPSSLSASVGDRVTITCRASQGIRNDLGWYQQKPGKAPKRLIYAASSLQSGVPSRFSGSGSGTEFTLTISSLQPEDFATYYCQQHNSYPWTFGQGTKVEIKR

SEQ ID NO: 39 - protein, 116 aa, Artificial Sequence
  Description of Artificial Sequence Synthetic polypeptide
EVQLLESGGGLVQPGGSLRLSCAASGFPFSTYAIHWVRQAPGKGLEWVSAISGSGGSTYYADSVKGRFTISRDNSKNTLYLQMNSLRAEDTAVYYCAGPDWYFDLWGRGTLVTVSS

SEQ ID NO: 40 - protein, 107 aa, Artificial Sequence
  Description of Artificial Sequence Synthetic polypeptide
DIQMTQSPSSLSASVGDRVTITCRASQAISNSLAWYQQKPGKAPKLLIYAASTLQRGVPSRFSGSGSGTDFTLTISSLQPEDFATYYCQQYYSTPYTFGQGTKLEIK

SEQ ID NO: 41 - protein, 107 aa, Artificial Sequence; identical to SEQ ID NO: 40
  Description of Artificial Sequence Synthetic polypeptide

SEQ ID NO: 42 - protein, 107 aa, Artificial Sequence
  Description of Artificial Sequence Synthetic polypeptide
DIQMTQSPSSLSASVGDRVTITCQASQDISNYLNWYQQKPGKAPKLLIFDASSLEAGVPSRFSGSGSGTDFTLTISSLQPEDFATYYCQQANSFPPTFGQGTEVEIK

SEQ ID NO: 43 - protein, 116 aa, Artificial Sequence
  Description of Artificial Sequence Synthetic polypeptide
QVQLVQSGAEVKKPGASVKVSCKASGYTFTSYAINWVRQAPGQGLEWMGILSPSGGGTSYAPKFQGRVTMTRDTSTSTVYMELSSLRSEDTAVYYCARGPWYFDLWGRGTLVTVSS

SEQ ID NO: 44 - protein, 107 aa, Artificial Sequence
  Description of Artificial Sequence Synthetic polypeptide
DIQMTQSPSSLSASVGDRVTITCRASQGISNSLAWYQQKPGKAPKLLIYAASSLQSGVPSRFSGSGSGTDFTLTISSLQPEDFATYYCQQSYSTPFTFGPGTKVDIK

SEQ ID NO: 45 - protein, 121 aa, Artificial Sequence
  Description of Artificial Sequence Synthetic polypeptide
QVQLQESGPGLVKPSQTLSLTCAVYGGSFSSGYWNWIRKHPGKGLEYIGYISYNGITYHNPSLKSRITINRDTSKNQYSLQLNSVTPEDTAVYYCARYKYDYDGGHAMDYWGQGTLVTVSS

SEQ ID NO: 46 - protein, 107 aa, Artificial Sequence
  Description of Artificial Sequence Synthetic polypeptide
DIQMTQSPSSLSASVGDRVTITCRASQDISNYLNWYQQKPGKAPKLLIYYTSKLHSGVPSRFSGSGSGTDYTLTISSLQPEDFATYYCQQGSALPWTFGQGTKVEIK

SEQ ID NO: 47 - protein, 117 aa, Artificial Sequence
  Description of Artificial Sequence Synthetic polypeptide
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRWYFSVWGQGTLVTVSS

SEQ ID NO: 48 - protein, 107 aa, Artificial Sequence
  Description of Artificial Sequence Synthetic polypeptide
DIQMTQSPSSLSASVGDRVTITCRASQDISNYLNWYQQKPGKAPKLLIYYTSRLRSGVPSRFSGSGSGTDFTLTISSLQPEDFATYYCQQGHTLPPTFGQGTKVEIK

SEQ ID NO: 49 - protein, 122 aa, Artificial Sequence
  Description of Artificial Sequence Synthetic polypeptide
EVQLVQSGAEVKKPGESLKISCKGSGYSFTSYWIGWVRQMPGKGLEWMGIIYPGDSDTRYSPSFQGQVTISADKSITTAYLQWSSLKASDTAMYYCARHPSYGSGSPNFDYWGQGTLVTVSS

SEQ ID NO: 50 - protein, 112 aa, Artificial Sequence
  Description of Artificial Sequence Synthetic polypeptide
DIVMTQTPLSSPVTLGQPASISCRSSQSLVYSDGNTYLSWLQQRPGQPPRLLIYKISNRFSGVPDRFSGSGAGTDFTLKISRVEAEDVGVYYCVQATQFPLTFGGGTKVEIK

SEQ ID NO: 51 - protein, 453 aa, Homo sapiens [sequence omitted]

SEQ ID NO: 52 - protein, 453 aa, Homo sapiens
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 53 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 54 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 55 - protein, 764 aa, Homo sapiens
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 56 - protein, 585 aa, Homo sapiens
KSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPR

SEQ ID NO: 57 - protein, 114 aa, Artificial Sequence
  Description of Artificial Sequence Synthetic polypeptide
DWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 58 - protein, 114 aa, Artificial Sequence
  Description of Artificial Sequence Synthetic polypeptide
NWVDVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 59 - protein, 114 aa, Artificial Sequence
  Description of Artificial Sequence Synthetic polypeptide
NWVNVISNLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 60 - protein, 114 aa, Artificial Sequence
  Description of Artificial Sequence Synthetic polypeptide
NWVNVISDLKKIEDLIQSMHIDATLYTESNVHPSCKVTAMKCFLLELQVISLESGDASIHDTVENLIILANNSLSSNGNVTESGCKECEELEEKNIKEFLQSFVHIVQMFINTS

SEQ ID NO: 61 - protein, 114 aa, Artificial Sequence
  Description of Artificial Sequence Synthetic polypeptide
NWVNVISDLKKIEDLIQSMHIDATLYTESDVHPSCKVTAMKCFLLELQVISLESGDASIHNTVENLIILANNSLSSNGNVTESGCKECEELEEKNIKEFLQSFVHIVQMFINTS

SEQ ID NO: 62 - protein, 114 aa, Artificial Sequence
  Description of Artificial Sequence Synthetic polypeptide
NWVNVISDLKKIEDLIQSMHIDATLYTESDVHPSCKVTAMKCFLLELQVISLESGDASIHDTVQNLIILANNSLSSNGNVTESGCKECEELEEKNIKEFLQSFVHIVQMFINTS

SEQ ID NO: 63 - protein, 114 aa, Artificial Sequence
  Description of Artificial Sequence Synthetic polypeptide
NWVNVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTS

SEQ ID NO: 64 - protein, 114 aa, Artificial Sequence
  Description of Artificial Sequence Synthetic polypeptide
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 65 - protein, 114 aa, Artificial Sequence
  Description of Artificial Sequence Synthetic polypeptide
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVEMFINTS

SEQ ID NO: 66 - protein, 114 aa, Artificial Sequence
  Description of Artificial Sequence Synthetic polypeptide
NWVDVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTS

SEQ ID NO: 67 - protein, 114 aa, Artificial Sequence
  Description of Artificial Sequence Synthetic polypeptide
DWVNVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTS

SEQ ID NO: 68 - protein, 114 aa, Artificial Sequence
  Description of Artificial Sequence Synthetic polypeptide
NWVNVISDLKKIEDLIQSMHIDATLYTESNVHPSCKVTAMKCFLLELQVISLESGDASIHDTVQNLIILANDSLSSNGNVTESGCKECEELEEKNIKEFLQSFVHIVQMFINTS

SEQ ID NO: 69 - protein, 159 aa, Macaca fascicularis
MKNHLLFWGVLAIFVKAVHVKAQEGERIVLVDNKCKCARITSRIIRSSEDPNEDIVERHIRIIVPLNNRENISDPTSPLRTKFVYHLSDLCKKCDPTEVELDNQIVTATQSNICDEDSATETCYTYDRNKCYTAVVPLTYGGETKMVQTALTPDSCYPD

SEQ ID NO: 70 - protein, 159 aa, Mus musculus
MKTHLLLWGVLAIFVKAVLVTGDDEATILADNKCMCTRVTSRIIPSTEDPNEDIVERNIRIVVPLNNRENISDPTSPLRRNFVYHLSDVCKKCDPVEVELEDQVVTATQSNICNEDDGVPETCYMYDRNKCYTTMVPLRYHGETKMVQAALTPDSCYPD

SEQ ID NO: 71 - protein, 161 aa, Macaca fascicularis
IFQKPHLRSVSIHCYLCLLLNSHFLTEAGIHVFILGCFSAGLPKTEANWVNVISDLKKIEDLIQSMHIDATLYTESDVHPSCKVTAMKCFLLELQVISHESGDTDIHDTVENLIILANNILSSNGNITESGCKECEELEEKNIKEFLQSFVHIVQMFINTS

SEQ ID NO: 72 - protein, 162 aa, Mus musculus
MKILKPYMRNTSISCYLCFLLNSHFLTEAGIHVFILGCVSVGLPKTEANWIDVRYDLEKIESLIQSIHIDTTLYTDSDFHPSCKVTAMNCFLLELQVILHEYSNMTLNETVRNVLYLANSTLSSNKNVAESGCKECEELEEKTFTEFLQSFIRIVQMFINTS

SEQ ID NO: 73 - protein, 239 aa, Macaca fascicularis
DHGITCPPPVSVEHADIRVKSYSLYSRERYICNSGFKRKAGTSSLTECVLNKATNIAHWTTPSLKCIRDPLLARQRPAPPFTVTTAGVTPQPESLSPSGKEPAASSPSSNTTAATTAAIVPSSRLMPSTSSSTGTTEIGSHESSHGPSQTTAKTWELTASASHQPPGVYPQGHSDTTVAISTSTVLLCGLSAVSLLACYIKSRQTAPPASIEMEAMEALPVTGETSSRDEDLENCSHDL

SEQ ID NO: 74 - protein, 263 aa, Mus musculus
MASPQLRGYGVQAIPVLLLLLLLLLLPLRVTPGTTCPPPVSIEHADIRVKNYSVNSRERYVCNSGFKRKAGTSTLIECVINKNTNVAHWTTPSLKCIRDPSLAHYSPVPTVVTPKVTSQPESPSPSAKEPEAFSPKSDTAMTTETAIMPGSRLTPSQTTSAGTTGTGSHKSSRAPSLAATMTLEPTASTSLRITEISPHSSKMTKVAISTSVLLVGAGVVMAFLAWYIKSRQPSQPCRVEVETMETVPMTVRASSKEDEDTGA

SEQ ID NO: 75 - protein, 118 aa, Artificial Sequence
  Description of Artificial Sequence Synthetic polypeptide
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSA

SEQ ID NO: 76 - protein, 108 aa, Artificial Sequence
  Description of Artificial Sequence Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 77 - protein, 361 aa, Artificial Sequence
  Description of Artificial Sequence Synthetic polypeptide
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSCPPPMSVEHADIWVKSYSLYSRERYICNSGFKRKAGTSSLTECVLNKATNVAHWTTPSLKCIRDPALVHQRPAPPSGGSGGGGSGGGSGGGGSLQDWVDVISNLKKIEDLIQSMHIDATLYTESNVHPSCKVTAMKCFLLELQVISLESGDASIHNTVQDLIILANDSLSSNGNVTESGCKECEELEEKNIKEFLQSFVHIVEMFINTS

SEQ ID NO: 78 - protein, 15 aa, Artificial Sequence
  Description of Artificial Sequence Synthetic peptide
GGGGSGGGGSGGGGS

SEQ ID NO: 79 - protein, 19 aa, Artificial Sequence
  Description of Artificial Sequence Synthetic peptide
GGSGGGGSGGGSGGGGSLQ

SEQ ID NO: 80 - protein, 5 aa, Artificial Sequence
  Description of Artificial Sequence Synthetic peptide
GGGGS

SEQ ID NO: 81 - protein, 10 aa, Artificial Sequence
  Description of Artificial Sequence Synthetic peptide
GGGGSGGGGS

SEQ ID NO: 82 - protein, 20 aa, Artificial Sequence
  Description of Artificial Sequence Synthetic peptide
GGGGSGGGGSGGGGSGGGGS

SEQ ID NO: 83 - protein, 25 aa, Artificial Sequence
  Description of Artificial Sequence Synthetic peptide
GGGGSGGGGSGGGGSGGGGSGGGGS

SEQ ID NO: 84 - protein, 737 aa, Artificial Sequence
  Description of Artificial Sequence Synthetic polypeptide
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPDGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFKQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGPKLVAASQAALGL

SEQ ID NO: 85 - protein, 361 aa, Artificial Sequence
  Description of Artificial Sequence Synthetic polypeptide
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNILSSNGNVTESGCKECEELEEKNIKEFLQSFVHIVQMFINTS

SEQ ID NO: 86 - protein, 361 aa, Artificial Sequence
  Description of Artificial Sequence Synthetic polypeptide
QEDERIVLVDNKCKCARITSRIIRSSEDPNEDIVERNIRIIVPLNNREDISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 87 - protein, 361 aa, Artificial Sequence
  Description of Artificial Sequence Synthetic polypeptide
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILADNSLSSNGNVTESGCKECEELEEKNIKEFLQSFVHIVQMFINTS

SEQ ID NO: 88 - protein, 361 aa, Artificial Sequence
  Description of Artificial Sequence Synthetic polypeptide
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGDVTESGCKECEELEEKNIKEFLQSFVHIVQMFINTS

SEQ ID NO: 89 - protein, 361 aa, Artificial Sequence
  Description of Artificial Sequence Synthetic polypeptide
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFIDTS

SEQ ID NO: 90 - protein, 361 aa, Artificial Sequence
  Description of Artificial Sequence Synthetic polypeptide
QEDERIVLVDNKCKCARITSRIIRSSEDPNEDIVERNIRIIVPLNNREDISDPTSPLRTRFVYHLSDLCKKCDPTEVELDNQIVTATQSNICDEDSATETCATYDRNKCYTAVVPLVYGGETKMVETALTPDACYPDGGGGSGGGGSGGGGSCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILADNSLSSNGDVTESGCKECEELEEKNIKEFLQSFVHIVQMFIDTS

SEQ ID NO: 91 - protein, 117 aa, Artificial Sequence
  Description of Artificial Sequence Synthetic polypeptide
QVQLQESGPGLVKPSETLSLTCTVSGFSLTSYDISWIRQPPGKGLEWLGVIWTGVGTNYNPSLKSRVTISKDTSKNQFSLKLSSVTAADTAVYYCARDPYYYGMDYWGQGTLVTVSS

SEQ ID NO: 92 - protein, 117 aa, Artificial Sequence
  Description of Artificial Sequence Synthetic polypeptide
QVQLQESGPGLVKPSETLSITCTVSGFSLTSYDISWVRQPPGKGLEWLGVIWTGVGTNYNPSFKSRLTISKDTSKNQVSLKMSSLTAADTAVYYCVRDPYYYGMDYWGQGTLVTVSS

SEQ ID NO: 93 - protein, 117 aa, Artificial Sequence
  Description of Artificial Sequence Synthetic polypeptide
QVQLQESGPGLVKPSETLSITCTVSGFSLTSYDISWIRQPPGKGLEWLGVIWTGVGTNYNPSFKSRLTISKDNSKNQVSLKMSSLTAADTAVYYCVRDPYYYGMDYWGQGTLVTVSS

SEQ ID NO: 94 - protein, 107 aa, Artificial Sequence
  Description of Artificial Sequence Synthetic polypeptide
DIQMTQSPSSLSASVGDRITITCRASQDISIWLSWYQQKPGKAPKLLIYKASNLHTGVPSRFSGSGSGTDFTLTISSLQPEDFATYYCLQSQSFPRTFGQGTKLEIK

SEQ ID NO: 95 - protein, 570 aa, Artificial Sequence
  Description of Artificial Sequence Synthetic polypeptide
QVQLQESGPGLVKPSETLSLTCTVSGFSLTSYDISWIRQPPGKGLEWLGVIWTGVGTNYNPSLKSRVTISKDTSKNQFSLKLSSVTAADTAVYYCARDPYYYGMDYWGQGTLVTVSSGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLDQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 96 - protein, 120 aa, Artificial Sequence
  Description of Artificial Sequence Synthetic polypeptide
EVQLVQSGAEVKKPGATVKISCKVSGYTFTSYWMYWVRQATGQGLEWMGRIDPNSGSTKYNEKFKNRVTITADKSTSTAYMELSSLRSEDTAVYYCARDYRKGLYAMDYWGQGTTVTVSS

SEQ ID NO: 97 - protein, 107 aa, Artificial Sequence
  Description of Artificial Sequence Synthetic polypeptide
DVVMTQSPLSLPVTLGQPASISCKASQDVGTAVAWYQQKPGQAPRLLIYWASTRHTGVPSRFSGSGSGTEFTLTISSLQPDDFATYYCQQYNSYPLTFGQGTKVEIK

SEQ ID NO: 98 - protein, 120 aa, Artificial Sequence
  Description of Artificial Sequence Synthetic polypeptide
EVQLVQSGAEVKKPGESLRISCKGSGYTFTSYWMYWVRQAPGQGLEWMGRIDPNSGSTKYNEKFKNRVTISVDTSKNQFSLKLSSVTAADTAVYYCARDYRKGLYAMDYWGQGTTVTVSS

SEQ ID NO: 99 - protein, 107 aa, Artificial Sequence
  Description of Artificial Sequence Synthetic polypeptide
EIVLTQSPDFQSVTPKEKVTITCKASQDVGTAVAWYLQKPGQSPQLLIYWASTRHTGVPSRFSGSGSGTDFTFTISSLQPEDIATYYCQQYNSYPLTFGQGTKVEIK

SEQ ID NO: 100 - protein, 120 aa, Artificial Sequence
  Description of Artificial Sequence Synthetic polypeptide
EVQLVQSGAEVKKPGATVKISCKVSGYTFTSYWMYWVRQARGQRLEWIGRIDPNSGSTKYNEKFKNRFTISRDNSKNTLYLQMNSLRAEDTAVYYCARDYRKGLYAMDYWGQGTTVTVSS

SEQ ID NO: 101 - protein, 107 aa, Artificial Sequence
  Description of Artificial Sequence Synthetic polypeptide
AIQLTQSPSSLSASVGDRVTITCKASQDVGTAVAWYLQKPGQSPQLLIYWASTRHTGVPSRFSGSGSGTDFTFTISSLEAEDAATYYCQQYNSYPLTFGQGTKVEIK

SEQ ID NO: 102 - protein, 115 aa, Artificial Sequence
  Description of Artificial Sequence Synthetic polypeptide
EVQLVQSGAEVKKPGASVKVSCKASGYTFTSYVMHWVRQAPGQRLEWIGYVNPFNDGTKYNEMFKGRATLTSDKSTSTAYMELSSLRSEDTAVYYCARQAWGYPWGQGTLVTVSS

SEQ ID NO: 103 - protein, 111 aa, Artificial Sequence
  Description of Artificial Sequence Synthetic polypeptide
DIVLTQSPATLSLSPGERATLSCRATESVEYYGTSLVQWYQQKPGQPPKLLIYAASSVDSGVPSRFSGSGSGTDFTLTINSLEAEDAAMYFCQQSRRVPYTFGQGTKLEIK

SEQ ID NO: 104 - protein, 115 aa, Artificial Sequence
  Description of Artificial Sequence Synthetic polypeptide
EVQLVQSGAEVKKPGASVKMSCKASGYTFTSYVMHWVKQAPGQRLEWIGYVNPFNDGTKYNEMFKGRATLTSDKSTSTAYMELSSLRSEDTAVYYCARQAWGYPWGQGTLVTVSS

SEQ ID NO: 105 - protein, 111 aa, Artificial Sequence
  Description of Artificial Sequence Synthetic polypeptide
DIVLTQSPATLSLSPGERATLSCRATESVEYYGTSLVQWYQQKPGQPPKLLIYAASSVDSGVPSRFSGSGSGTDFTLTINSLEAEDAATYFCQQSRRVPYTFGQGTKLEIK

SEQ ID NO: 106 - protein, 115 aa, Artificial Sequence; identical to SEQ ID NO: 104
  Description of Artificial Sequence Synthetic polypeptide

SEQ ID NO: 107 - protein, 111 aa, Artificial Sequence
  Description of Artificial Sequence Synthetic polypeptide
DIVLTQSPASLALSPGERATLSCRATESVEYYGTSLVQWYQQKPGQPPKLLIYAASSVDSGVPSRFSGSGSGTDFTLTINSLEEEDAAMYFCQQSRRVPYTFGQGTKLEIK

SEQ ID NO: 108 - protein, 115 aa, Artificial Sequence; identical to SEQ ID NO: 104
  Description of Artificial Sequence Synthetic polypeptide

SEQ ID NO: 109 - protein, 111 aa, Artificial Sequence; identical to SEQ ID NO: 103
  Description of Artificial Sequence Synthetic polypeptide

SEQ ID NO: 110 - protein, 115 aa, Artificial Sequence
  Description of Artificial Sequence Synthetic polypeptide
EVQLQQSGPELVKPGASVKMSCKASGYTFTSYVMHWVKQKPGQGLEWIGYVNPFNDGTKYNEMFKGKATLTSDKSSSTAYMELSSLTSEDSAVYYCARQAWGYPWGQGTLVTVSA

SEQ ID NO: 111 - protein, 111 aa, Artificial Sequence
  Description of Artificial Sequence Synthetic polypeptide
DIVLTQSPASLAVSLGQRATISCRATESVEYYGTSLVQWYQQKPGQPPKLLIYAASSVDSGVPARFSGSGSGTDFSLTIHPVEEDDIAMYFCQQSRRVPYTFGGGTKLEIK

SEQ ID NO: 112 - protein, 118 aa, Artificial Sequence
  Description of Artificial Sequence Synthetic polypeptide
EVQLVQSGAEVKKPGASVKVSCKASGYTFTGYYMHWVRQAPGQGLEWMGWINPNSDNTGSAQKFQGRVFMTKTTSLNTAYMELSGLRSEDTAIYYCARERSSGYFDFWGQGTLVTVSS

SEQ ID NO: 113 - protein, 107 aa, Artificial Sequence
  Description of Artificial Sequence Synthetic polypeptide
DIVMTQSPSSLSASVGDRVTITCRASQSISSYLNWYQQKPGKAPKLLIYAASSLQSGVPSRFSGSGSGTDFTLTISSLQPEDFATYYCQQSYSTPITFGQGTRLEIK

SEQ ID NO: 114 - protein, 124 aa, Artificial Sequence
  Description of Artificial Sequence Synthetic polypeptide
QVQLVQSGAEVKKPGASVKVSCKTSGNTFTNYYMHWVRQAPGQGLEWMGIMNPSGGSTSYAQKFQGRVTMTRDKSTSTVYMELSSLTSEDTAVYYCARDLFPHIYGNYYGMDIWGQGTTVTVSS

SEQ ID NO: 115 - protein, 107 aa, Artificial Sequence
  Description of Artificial Sequence Synthetic polypeptide
DIVMTQSPPSLSASVGDRVTITCRASQSISSYLNWYQQKPGKAPKLLIYAASSLQSGVPSRFSGSGSGTDFTLTISSLQPEDFATYYCQQSYSTPYTFGQGTKVEIK

SEQ ID NO: 116 - protein, 121 aa, Artificial Sequence
  Description of Artificial Sequence Synthetic polypeptide
QMQLVQSGAEVKKPGSSVKVSCKASGGTFSSYAISWVRQAPGQGLEWMGGIIPIFGTANYAQKFQGRVTITADESTSTAYMELSSLRSEDTAVYYCARGNIVATITPLDYWGQGTLVTVSS

SEQ ID NO: 117 - protein, 108 aa, Artificial Sequence
  Description of Artificial Sequence Synthetic polypeptide
SYELMQPPSVSVAPGKTATIACGGENIGRKTVHWYQQKPGQAPVLVIYYDSDRPSGIPERFSGSNSGNTATLTISRVEAGDEADYYCQVWDSSSDHRIFGGGTKLTVL

SEQ ID NO: 118 - protein, 118 aa, Artificial Sequence
  Description of Artificial Sequence Synthetic polypeptide
EVQLVQSGGGLVQPGGSLRLSCAASGFTFSSYSMNWVRQAPGKGLEWVSYISSSSSTIYYADSVKGRFTISRDNAKNSLYLQMNSLRDEDTAVYYCARGDYYYGMDVWGQGTTVTVSS

SEQ ID NO: 119 - protein, 108 aa, Artificial Sequence
  Description of Artificial Sequence Synthetic polypeptide
EIVLTQSPSSLSASIGDRVTLTCRASQSIRRFLNWYQQKPGKAPELLIYTASSLQSGVPSRFSGSGSGTDFTLTINSLQPEDFATYYCQQSYAVSPYTFGQGTKVEIR

SEQ ID NO: 120 - protein, 119 aa, Artificial Sequence
  Description of Artificial Sequence Synthetic polypeptide
EVQLVESGAEVKKPGSSVKVSCKASGGTFSSYAISWVRQAPGQGLEWMGGIIPIFGTANYAQKFQGRVTITADESTSTAYMELSSLRSEDTAVYYCARAPYYYYYMDVWGQGTTVTVSS

SEQ ID NO: 121 - protein, 109 aa, Artificial Sequence
  Description of Artificial Sequence Synthetic polypeptide
QSALTQPASVSGSLGQSVTISCTGSSSDVGSYNLVSWYQQHPGKAPNLMIYDVSKRSGVSNRFSGSKSGNTASLTISGLQAEDEADYYCSSYTGISTVVFGGGTKLTVL

SEQ ID NO: 122 - protein, 121 aa, Artificial Sequence
  Description of Artificial Sequence Synthetic polypeptide
QMQLVQSGAEVKKPGSSVKVSCKASGGTFSSYAYSWVRQAPGQGLEWMGGIIPSFGTANYAQKFQGRVTITADESTSTAYMELSSLRSEDTAVYYCARGPIVATITPLDYWGQGTLVTVSS

SEQ ID NO: 123 - protein, 108 aa, Artificial Sequence
  Description of Artificial Sequence Synthetic polypeptide
SYELMQPPSVSVAPGKTATIACGGENIGRKTVHWYQQKPGQAPVLVIYYDSDRPSGIPERFSGSNSGNTATLTISRVEAGDEADYYCLVWDSSSDHRIFGGGTKLTVL

SEQ ID NO: 124 - protein, 121 aa, Artificial Sequence
  Description of Artificial Sequence Synthetic polypeptide
QVQLQQSGAEVKKPGSSVKVSCKASGGTFSSYAISWVRQAPGQGLEWMGRIIPILGIANYAQKFQGRVTITADKSTSTAYMELSSLRSEDTAVYYCARGGSYGSLYAFDIWGQGTMVTVSS

SEQ ID NO: 125 - protein, 110 aa, Artificial Sequence
  Description of Artificial Sequence Synthetic polypeptide
QSVLTQPPSVSAAPGQRVSISCSGRSSNIASHDVFWYQQLPGTAPKIVMYETNKRPWGIPDRFSGSKSGTSATLDIAGLQTGDEADYYCGAWDSGLTGMLFGGGTKVTVL

SEQ ID NO: 126 - protein, 121 aa, Artificial Sequence
  Description of Artificial Sequence Synthetic polypeptide
QVQLQQSGAEVKKPGSSVKVSCKASGGTFSSYAISWVRQAPGQGLEWMGRIIPILGIANYAQKFQGRVTITADKSTSTAYMELSSLRSEDTAVYYCARGGSYGSLYAFDIWGQGTTVTVSS

SEQ ID NO: 127 - protein, 105 aa, Artificial Sequence
  Description of Artificial Sequence Synthetic polypeptide
SYELTQPPSVSVAPGKTTRITCGGDNIGRKSVHWYQQRPGQAPLLLVYDDGDRPSGIPDRFSGSNSGNTATLTISGTQAMDEADYYCQAWDSTVVFGGGTRLTVL

SEQ ID NO: 128 - protein, 122 aa, Artificial Sequence
  Description of Artificial Sequence Synthetic polypeptide
QVQLQQSGAEVKKPGSSVKVSCKASGGTFSSYAISWVRQAPGQGLEWMGRIIPILGIANYAQKFQGRVTITADKSTSTAYMELSSLRSEDTAVYYCARGGYGGNSLYAFDIWGQGTMVTVSS

SEQ ID NO: 129 - protein, 110 aa, Artificial Sequence
  Description of Artificial Sequence Synthetic polypeptide
QSVVTQPPSVSAAPGQKVTISCSGSSSNIGNNYVSWYQQLPGTAPKLLIYDNNERLSGIPDRFSGSKSGTSATLGISGLQTGDEADYYCGTWDSSLSVVVFGGGTKLTVL

SEQ ID NO: 130 - protein, 116 aa, Artificial Sequence
  Description of Artificial Sequence Synthetic polypeptide
EVQLVQSGAEVKKPGASVKVSCKASGGTFSSYAISWVRQAPGQGLEWMGRIIPILGIANYAQKFQGRVTITADKSTSTAYMELSSLRSEDTAVYYCARSGHGYSYGAFDYWGQGTL

SEQ ID NO: 131 - protein, 113 aa, Artificial Sequence
  Description of Artificial Sequence Synthetic polypeptide
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGSYVVFGGGTKLTVL

SEQ ID NO: 132 - protein, 121 aa, Artificial Sequence
  Description of Artificial Sequence Synthetic polypeptide
QVQLVQSGAEVKKPGSSVKVSCKASGGTFSSYAISWVRQAPGQGLEWMGRIIPILGIANYAQKFQGRVTITADKSTSTAYMELSSLRSEDTAVYYCARSGHGYSYGAFDYWGQGTLVTVSS

SEQ ID NO: 133 - protein, 113 aa, Artificial Sequence; identical to SEQ ID NO: 131
  Description of Artificial Sequence Synthetic polypeptide

SEQ ID NO: 134 - protein, 117 aa, Artificial Sequence
  Description of Artificial Sequence Synthetic polypeptide
QVQLKESGPGLVAPSQSLSITCTVSGFSLTSYGVHWVRQPPGKGLEWLGVIWAGGSTNYNSALMSRLSISKDNSKSQVVLKMNSLQTDDTAMYYCAKPYGNSAMDYWGQGTSVTVSS

SEQ ID NO: 135 - protein, 108 aa, Artificial Sequence
  Description of Artificial Sequence Synthetic polypeptide
DILMTQSHKFMSTSVGDTVSITCKASQDVGIVVAWYQQKPGQSPKLLIYWASIRHTGVPDRFTGSGSGTDFTLTISNVQSEDLADYFCQQYSNYPLYTFGGGTKLEIK

SEQ ID NO: 136 - protein, 117 aa, Artificial Sequence
  Description of Artificial Sequence Synthetic polypeptide
EVQLVESGGGLVQPGGSLRLSCAVSGFSLTSYGVHWVRQAPGKGLEWVAVIWAGGSTNYNSALMSRLTISKDNAKNSVYLQMNSLRAEDTAVYYCAKPYGNSAMDYWGQGTLVTVSS

SEQ ID NO: 137 - protein, 108 aa, Artificial Sequence
  Description of Artificial Sequence Synthetic polypeptide
DIQMTQSPSTLSASVGDRVTITCKASQDVGIVVAWYQQKPGKAPKLLIYWASIRHTGVPSRFSGSGSGTEFTLTISSLQPDDFATYYCQQYSNYPLYTFGQGTKLEIK

SEQ ID NO: 138 - protein, 117 aa, Artificial Sequence
  Description of Artificial Sequence Synthetic polypeptide
EVQLVESGGGLVQPGGSLRLSCAVSGFSLTSYGVHWVRQAPGKGLEWVAVIWAGGSTNYVDSVKGRFTISKDNAKNSVYLQMNSLRAEDTAVYYCAKPYGNSAMDYWGQGTLVTVSS

SEQ ID NO: 139 - protein, 108 aa, Artificial Sequence; identical to SEQ ID NO: 137
  Description of Artificial Sequence Synthetic polypeptide

SEQ ID NO: 140 - protein, 117 aa, Artificial Sequence
  Description of Artificial Sequence Synthetic polypeptide
EVQLVESGGGLVQPGGSLRLSCAVSGFSLTSYGVHWVRQAPGKGLEWVAVIWAGGSTNYVDSVKGRLTISKDNAKNTVYLQMNSLRAEDTAVYYCAKPYGNSAMDYWGQGTLVTVSS

SEQ ID NO: 141 - protein, 108 aa, Artificial Sequence
  Description of Artificial Sequence Synthetic polypeptide
DIQMTQSPSSLSASVGDRVTITCKASQDVGIVVAWYQQKPGKAPKLLIYWASIRHTGVPSRFSGSGSGTEFTLTISSLQPDDFATYYCQQYSNYPLYTFGQGTKVEIK

SEQ ID NO: 142 - protein, 117 aa, Artificial Sequence
  Description of Artificial Sequence Synthetic polypeptide
EVQLVESGGGLVQPGGSLRLSCAVSGFSLTSYGVHWVRQAPGKGLEWVAVIWAGGSTNYADSVKGRFTISKDNSKNTVYLQMNSLRAEDTAVYYCAKPYGNSAMDYWGQGTLVTVSS

SEQ ID NO: 143 - protein, 108 aa, Artificial Sequence; identical to SEQ ID NO: 141
  Description of Artificial Sequence Synthetic polypeptide

SEQ ID NO: 144 - protein, 117 aa, Artificial Sequence
  Description of Artificial Sequence Synthetic polypeptide
EVQLVESGGGLVQPGGSLRLSCAVSGFSLTSYGVHWVRQAPGKGLEWVAVIWAGGSTNYADSVKGRFTISKDTSKNTVYLQMNSLRAEDTAVYYCAKPYGNSAMDYWGQGTLVTVSS

SEQ ID NO: 145 - protein, 108 aa, Artificial Sequence; identical to SEQ ID NO: 141
  Description of Artificial Sequence Synthetic polypeptide

SEQ ID NO: 146 - protein, 117 aa, Artificial Sequence
  Description of Artificial Sequence Synthetic polypeptide
EVQLVESGGGLVQPGGSLRLSCAVSGFSLTSYGVHWVRQAPGKGLEWVAVIWAGGSTNYVDSVKGRFTISKDNAKNTVYLQMNSLRAEDTAVYYCAKPYGTSAMDYWGQGTLVTVSS

SEQ ID NO: 147 - protein, 108 aa, Artificial Sequence; identical to SEQ ID NO: 141
  Description of Artificial Sequence Synthetic polypeptide

SEQ ID NO: 148 - protein, 117 aa, Artificial Sequence
  Description of Artificial Sequence Synthetic polypeptide
EVQLVESGGGLVQPGGSLRLSCAVSGFSLTSYGVHWVRQAPGKGLEWVAVIWAGGSTNYADSVKGRFTISKDTSKNTVYLQMNSLRAEDTAVYYCAKPYGTSAMDYWGQGTLVTVSS

SEQ ID NO: 149 - protein, 108 aa, Artificial Sequence; identical to SEQ ID NO: 141
  Description of Artificial Sequence Synthetic polypeptide

SEQ ID NO: 150 - protein, 116 aa, Artificial Sequence
  Description of Artificial Sequence Synthetic polypeptide
EVQLVESGGGLVKPGGSLRLSCAASGFIFRSYGMSWVRQAPGKGLEWVASISSGGSTYYPDSVKGRFTISRDNAKNSLYLQMNSLRAEDTAVYDCARGYDSGFAYWGQGTLVTVSS

SEQ ID NO: 151 - protein, 111 aa, Artificial Sequence
  Description of Artificial Sequence Synthetic polypeptide
DIVLTQSPASLAVSPGQRATITCRASQSVSTSSSSFMHWYQQKPGQPPKLLIKYASNLESGVPARFSGSGSGTDFTLTINPVEANDTANYYCQHSWEIPYTFGQGTKLEIK

SEQ ID NO: 152 - protein, 116 aa, Artificial Sequence
  Description of Artificial Sequence Synthetic polypeptide
QITLKESGPTLVKPTQTLTLTCTVSGFSLSTYGVHWIRQPPGKALEWLGVIWRGVTTDYNAAFMSRLTITKDNSKNQVVLTMNNMDPVDTATYYCARLGFYAMDYWGQGTLVTVSS

SEQ ID NO: 153 - protein, 107 aa, Artificial Sequence
  Description of Artificial Sequence Synthetic polypeptide
DIQMTQSPSSLSASVGDRVTITCKASQSVSNDVAWYQQKPGKAPKLLIYYAANRYTGVPDRFSGSGYGTDFTFTISSLQPEDIATYFCQQDYTSPYTFGQGTKLEIK

SEQ ID NO: 154 - protein, 116 aa, Artificial Sequence
  Description of Artificial Sequence Synthetic polypeptide
EVQLLESGGGLVQPGGSLRLSCAASGFTFSSYAMSWVRQAPGKGLEWVSSIWRNGIVTVYADSVKGRFTISRDNSKNTLYLQMNSLRAEDTAVYYCAKWSAAFDYWGQGTLVTVSS

SEQ ID NO: 155 - protein, 108 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQDNGYPSTFGGGTKVEIKR

SEQ ID NO: 156 - protein, 121 aa, Artificial Sequence
  Description of Artificial Sequence Synthetic polypeptide
EVQLVQSGAEVKKPGASVKVSCKASGYTFTKYIIHWVRQAPGQGLEWMGWFYPGSGNIRYNEKIKGRVTMTRDTSTSTVYMELSSLRSEDTAVYYCARHGELGGGYFFDYWGQGTTVTVSS

SEQ ID NO: 157 - protein, 107 aa, Artificial Sequence
  Description of Artificial Sequence Synthetic polypeptide
DIQLTQSPSFLSASVGDRVTITCRASSSVSNIHWYQQKPGKAPKPWIYATSNLASGVPSRFSGSGSGTEFTLTISSLQPEDFATYYCQQWSSNPLTFGQGTKLEIKR

SEQ ID NO: 158 - protein, 121 aa, Artificial Sequence; identical to SEQ ID NO: 156
  Description of Artificial Sequence Synthetic polypeptide

SEQ ID NO: 159 - protein, 107 aa, Artificial Sequence
  Description of Artificial Sequence Synthetic polypeptide
DIQLTQSPSFLSASVGDRVTITCRASSKTGNIHWYQQKPGKAPKPWIYATSNLASGVPSRFSGSGSGTEFTLTISSLQPEDFATYYCQQWSSNPLTFGQGTKLEIKR

SEQ ID NO: 160 - protein, 121 aa, Artificial Sequence; identical to SEQ ID NO: 156
  Description of Artificial Sequence Synthetic polypeptide

SEQ ID NO: 161 - protein, 107 aa, Artificial Sequence
  Description of Artificial Sequence Synthetic polypeptide
DIQLTQSPSFLSASVGDRVTITCRASSGASNIHWYQQKPGKAPKPWIYATSNLASGVPSRFSGSGSGTEFTLTISSLQPEDFATYYCQQWSSNPLTFGQGTKLEIKR

SEQ ID NO: 162 - protein, 123 aa, Artificial Sequence
  Description of Artificial Sequence Synthetic polypeptide
EVQLVESGGGLVQPGRSLRLSCAASGFTFDDYAMHWVRQVPGKGLEWVSGISWIRTGIGYADSVKGRFTIFRDNAKNSLYLQMNSLRAEDTALYYCAKDMKGSGTYGGWFDTWGQGTLVTVSS

SEQ ID NO: 163 - protein, 107 aa, Artificial Sequence
  Description of Artificial Sequence Synthetic polypeptide
DIQMTQSPSSLSASVGDRVTITCRASQSISSYLNWYQQKPGKAPKLLIYVASSLQSGVPSRFSGSGSGTDFTLTISSLQPEDFATYYCQQSYSTPITFGQGTRLEIK

SEQ ID NO: 164 - protein, 123 aa, Artificial Sequence
  Description of Artificial Sequence Synthetic polypeptide
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARSPDYSPYYYYGMDVWGQGTTVTVSS

SEQ ID NO: 165 - protein, 111 aa, Artificial Sequence
  Description of Artificial Sequence Synthetic polypeptide
QSVLTQPPSASGTPGQRVTISCSGSSSNIGSNTVNWYQQLPGTAPKLLIYGNSNRPSGVPDRFSGSKSGTSASLAISGLQSEDEADYYCQSYDSSLSGSVFGGGIKLTVLG

SEQ ID NO: 166 - protein, 118 aa, Artificial Sequence
  Description of Artificial Sequence Synthetic polypeptide
QVQLQESGPGLVKPSQTLSLTCTVSGGSISNDYWTWIRQHPGKGLEYIGYISYTGSTYYNPSLKSRVTISRDTSKNQFSLKLSSVTAADTAVYYCARSGGWLAPFDYWGRGTLVTVSS

SEQ ID NO: 167 - protein, 113 aa, Artificial Sequence
  Description of Artificial Sequence Synthetic polypeptide
DIVMTQSPDSLAVSLGERATINCKSSQSLFYHSNQKHSLAWYQQKPGQPPKLLIYGASTRESGVPDRFSGSGSGTDFTLTISSLQAEDVAVYYCQQYYGYPYTFGGGTKVEIK

SEQ ID NO: 168 - protein, 119 aa, Artificial Sequence
  Description of Artificial Sequence Synthetic polypeptide
QVQLVQSGAEVKKPGASVKVSCKASGYTFTSYWMHWVRQAPGQGLEWMGRIGPNSGFTSYNEKFKNRVTMTRDTSTSTVYMELSSLRSEDTAVYYCARGGSSYDYFDYWGQGTTVTVSS

SEQ ID NO: 169 - protein, 111 aa, Artificial Sequence
  Description of Artificial Sequence Synthetic polypeptide
DIVLTQSPASLAVSPGQRATITCRASESVSIHGTHLMHWYQQKPGQPPKLLIYAASNLESGVPARFSGSGSGTDFTLTINPVEAEDTANYYCQQSFEDPLTFGQGTKLEIK

SEQ ID NO: 170 - protein, 120 aa, Artificial Sequence
  Description of Artificial Sequence Synthetic polypeptide
QVQLVQSGAEVKKPGASVKVSCKASGYTFTSYWMHWVRQAPGQGLEWMGAIYPGNSDTSYNQKFKGRVTMTRDTSTSTVYMELSSLRSEDTAVYYCTRWGYGFDGAMDYWGQGTLVTVSS

SEQ ID NO: 171 - protein, 108 aa, Artificial Sequence
  Description of Artificial Sequence Synthetic polypeptide
DIQMTQSPSSLSASVGDRVTITCRASQDIGSSLNWYQQKPGKAPKRLIYATSSLDSGVPSRFSGSGSGTEFTLTISSLQPEDFATYYCLQYASSPYTFGGGTKVEIKR

SEQ ID NO: 172 - protein, 120 aa, Artificial Sequence
  Description of Artificial Sequence Synthetic polypeptide
EVQLVESGGGLVQPGGSLRLSCAASGFTFSSYWMSWVRQAPGKGLEWVADIKQDGSEKYYVDSVKGRFTISRDNAKNSLYLQMNSLRAEDTAVYYCARDRLWFGGVMDAWGQGASVTVSS

SEQ ID NO: 173 - protein, 107 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYNSYSWTFGQGTKVEIK

SEQ ID NO: 174 - protein, 121 aa, Artificial Sequence
  Description of Artificial Sequence Synthetic polypeptide
EVQLVESGGGLVQPGGSLRLSCAASGITFSSYWMSWVRQTPGKGLEWVANIKQDGSEKYYVKSVKGRFTISRDNAKNSLYLQMNSLRAEDTAVYYCVRDRAVPGTGAFDIWGQGTMVIVSS

SEQ ID NO: 175 - protein, 107 aa, Artificial Sequence
  Description of Artificial Sequence Synthetic polypeptide
DIQMTQSPSTLSASVGDRVTITCRASQSISNWLAWYQQKPGKAPKLLIYKASSLESGVPSRFSGSGSGTEFTLTISSLQPDDFATYYCQQYDSSLFTFGQGTKLEIK

SEQ ID NO: 176 - protein, 119 aa, Artificial Sequence
  Description of Artificial Sequence Synthetic polypeptide
EVQLVESGGGLVQPGGSLRLSCAASGFTFSSYWMSWVRQAPGKGLEWVANIKQDGNEKYYVDSVKGRFTISRDNAKNSLYLQMNSLRAEDTAVYYCARNIKWGDAFDIWGQGTMVTVSS

SEQ ID NO: 177 - protein, 107 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDFAIYSCQQSYSIPLTFGGGTKVEIK

SEQ ID NO: 178 - protein, 121 aa, Artificial Sequence
  Description of Artificial Sequence Synthetic polypeptide
EVQLVESGGGLVQPGGSLRLSCAASGFTFNTYWMNWVRQAPGRGLEWVANIKQDGGEKYYVDSVKGRFTISRDNAKNSLYLQMNSLRAEDTAMYYCARDGRSWYPDAFDIWGQGTVVTVSS

SEQ ID NO: 179 - protein, 106 aa, Artificial Sequence
  Description of Artificial Sequence Synthetic polypeptide
DIQMTQSPSTLSASVGDRVTITCRASQSISTWLAWYQQKLGKAPKLLIYEVSILESGVPSRFSGSGSGTEFTLTISSLQPDDFATYYCQQYHSFSSFGGGTRVEIK

SEQ ID NO: 180 - protein, 121 aa, Artificial Sequence
  Description of Artificial Sequence Synthetic polypeptide
EVQLVESGGGLVQPGGSLRLSCVVSGFTFSSYWMSWVRQTPGKGLEWVANIKQDGSEKYYVDSVKGRLTISRDNAKNSLYLQMNSLRAEDTAVYYCVRDRAVPGTGALDIWGQGTMVTVSS

SEQ ID NO: 181 - protein, 107 aa, Artificial Sequence
  Description of Artificial Sequence Synthetic polypeptide
DIQMTQSPSTLSASVGDRVTITCRASQSISSWLAWYQQKPGKAPKLLIYKASSLESGVPSRFSGSGSGTEFTLTISSLQPDDFATYYCQQYNSYLFTFGQGTKLEIK

SEQ ID NO: 182 - protein, 119 aa, Artificial Sequence
  Description of Artificial Sequence Synthetic polypeptide
EVQLVESGGGLVQPGGSLRLSCVASGFTFSSYWMSWVRQAPGKGLEWVANIKQDGSEKYYVDSVKGRLTISRDNAKNSLYLQMKSLRAEDTAVYYCARDPIYSSYFDYWGQGILVTVSS

SEQ ID NO: 183 - protein, 107 aa, Artificial Sequence
  Description of Artificial Sequence Synthetic polypeptide
DIQMTQSPSSLSASVGDRVTITCRASQSISNYLNWYQQKPGKAPKLLIYAASSLQSGVPSRFSGSGSGTDFTLTISSLQPEDFAAYYCQQSYGIPFTFGPGTKVDIK

SEQ ID NO: 184 - protein, 120 aa, Artificial Sequence
  Description of Artificial Sequence Synthetic polypeptide
EVQLVESGGGLVQPGGSLRLSCAASGFTFSSYWMSWVRQAPGKGLEWVADIKQDGSEKYYVDSVKGRFTISRDNAKNSLYLQMNSLRAEDTAVYYCTRDKPHWGGVMDAWGQGTSVTVSS

SEQ ID NO: 185 - protein, 107 aa, Artificial Sequence
  Description of Artificial Sequence Synthetic polypeptide
DIQMTQSPSTLSASEGDRVTITCRASQSISRWLAWYQQKPGKAPKLLIYKASSLESGVPSRFSGSGSGTEFTLTISSLQPDDFATYYCQQYHSYSWTFGQGTKVEIK

SEQ ID NO: 186 - protein, 120 aa, Artificial Sequence
  Description of Artificial Sequence Synthetic polypeptide
EVQLVESGGGLVQPGGSLRLSCTVSGIDLSSYTMGWVRQAPGKGLEWVGIISSGGRTYYASWAKGRFTISRDTSKNTVYLQMNSLRAEDTAVYYCARGRYTGYPYYFALWGQGTLVTVSS

SEQ ID NO: 187 - protein, 113 aa, Artificial Sequence
  Description of Artificial Sequence Synthetic polypeptide
EIVMTQSPSTLSASVGDRVIITCQASEDIYSLLAWYQQKPGKAPKLLIYDASDLASGVPSRFSGSGSGAEFTLTISSLQPDDFATYYCQGNYGSSSSSSYGAVFGQGTKLEIK

SEQ ID NO: 188 - protein, 120 aa, Artificial Sequence
  Description of Artificial Sequence Synthetic polypeptide
QVQLKESGPGLVAPSQNLSITCTVSGFSLSNYDISWIRQPPGKGLEWLGVIWTGGATNYNSAFMSRLSISRDNSKSQVFLKMNSLQTDDTAIYYCVRDSNYRYDEPFTYWGQGTLVTVSA

SEQ ID NO: 189 - protein, 106 aa, Artificial Sequence
  Description of Artificial Sequence Synthetic polypeptide
DILLTQSPAILSVSPGERVSLSCRASQSIGTNIHWFQQRTNGSPRLLIKYASESISGIPSRFSGSGSGTDFTLSINSVESEDIADYYCQQSNSWPYTFGGGTKLEI

SEQ ID NO: 190 - protein, 120 aa, Artificial Sequence
  Description of Artificial Sequence Synthetic polypeptide
QVQLQESGPGLVKPSENLSITCTVSGFSLSNYDISWIRQPPGKGLEWLGVIWTGGATNYNPAFKSRLTISRDNSKSQVSLKMSSLQAADTAVYYCVRDSNYRYDEPFTYWGQGTLVTVSS

SEQ ID NO: 191 - protein, 107 aa, Artificial Sequence
  Description of Artificial Sequence Synthetic polypeptide
EIVLTQSPDTLSVTPKEKVTLTCRASQSIGTNIHWFQQRPGQSPKLLIKYASESISGIPSRFSGSGSGTDFTLTINSVEAEDAATYYCQQSNSWPYTFGQGTKLEIK

SEQ ID NO: 192 - protein, 120 aa, Artificial Sequence
  Description of Artificial Sequence Synthetic polypeptide
QVQLQESGPGLVKPSETLSITCTVSGFSLSNYDISWIRQPPGKGLEWLGVIWTGGATNYNPALKSRLTISRDNSKNQVSLKMSSVTAADTAVYYCVRDSNYRYDEPFTYWGQGTLVTVSS

SEQ ID NO: 193 - protein, 107 aa, Artificial Sequence
  Description of Artificial Sequence Synthetic polypeptide
EIVLTQSPDTLSVTPKEKVTLTCRASQSIGTNIHWFQQKPGQSPKLLIKYASESISGVPSRFSGSGSGTDFTLTINSVEAEDAATYYCQQSNSWPYTFGQGTKLEIK

SEQ ID NO: 194 - protein, 119 aa, Artificial Sequence
  Description of Artificial Sequence Synthetic polypeptide
EVQLVESGGGLVQPGGSLRLSCAASGFTFSSYDMSWVRQAPGKSLEWVATISDAGGYIYYSDSVKGRFTISRDNAKNSLYLQMNSLRDEDTAVYICAREFGKRYALDYWGQGTTVTVSS

SEQ ID NO: 195 - protein, 107 aa, Artificial Sequence
  Description of Artificial Sequence Synthetic polypeptide
DIQMTQSPSSLSASVGDRVTITCKASQDVTPAVAWYQQKPGKAPKLLIYSTSSRYTGVPSRFSGSGSGTDFTFTISSLQPEDIATYYCQQHYTTPLTFGQGTKLEIK

SEQ ID NO: 196 - protein, 120 aa, Artificial Sequence
  Description of Artificial Sequence Synthetic polypeptide
EVQLVQSGAEVKKPGSSVKVSCKASGGTFSRSAISWVRQAPGQGLEWMGVIIPAFGEANYAQKFQGRVTITADESTSTAYMELSSLRSEDTAVYYCARGRQMFGAGIDFWGQGTLVTVSS

SEQ ID NO: 197 - protein, 112 aa, Artificial Sequence
  Description of Artificial Sequence Synthetic polypeptide
NFMLTQPHSVSESPGKTVTISCTRSSGSIDSNYVQWYQQRPGSAPTTVIYEDNQRPSGVPDRFSGSIDSSSNSASLTISGLKTEDEADYYCQSYDSNNRHVIFGGGTKLTVL

SEQ ID NO: 198 - protein, 122 aa, Artificial Sequence
  Description of Artificial Sequence Synthetic polypeptide
EVQLVESGGGVVRPGGSLRLSCAASGFTFDDYAMSWVRQAPGKGLEWVSDISWSGSNTNYADSVKGRFTISRDNAKNSLYLQMNSLRAEDTALYHCARAPLLLAMTFGVGSWGQGTLVTVSS

SEQ ID NO: 199 - protein, 106 aa, Artificial Sequence
  Description of Artificial Sequence Synthetic polypeptide
QTVVTQEPSLSVSPGGTVTLTCGLSSGTVTAINYPGWYQQTPGQAPRTLIYNTNTRHSGVPDRFSGSISGNKAALTITGAQAEDEADYYCALYMGNGGHMFGGGTK

SEQ ID NO: 200 - protein, 117 aa, Artificial Sequence
  Description of Artificial Sequence Synthetic polypeptide
QVQLVQSGAEVKKPGASVKVSCKASGYTFTDYGFSWVRQAPGQGLEWMGWITAYNGNTNYAQKLQGRVTMTTDTSTSTVYMELRSLRSDDTAVYYCARDYFYGMDVWGQGTTVTVSS

SEQ ID NO: 201 - protein, 107 aa, Artificial Sequence
  Description of Artificial Sequence Synthetic polypeptide
EIVLTQSPATLSLSPGERATLSCRASQSVSSYLVWYQQKPGQAPRLLIYDASNRATGIPARFSGSGSGTDFTLTISSLEPEDFAVYYCQQRSNWPRTFGQGTKVEIK

SEQ ID NO: 202 - protein, 123 aa, Artificial Sequence
  Description of Artificial Sequence Synthetic polypeptide
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 203 - protein, 106 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 204 - protein, 118 aa, Artificial Sequence
  Description of Artificial Sequence Synthetic polypeptide
QVQLVQSGAEVKKPGASVKVSCKASGYTFTSYDVHWVRQAPGQRLEWMGWLHADTGITKFSQKFQGRVTITRDTSASTAYMELSSLRSEDTAVYYCARERIQLWFDYWGQGTLVTVSS

SEQ ID NO: 205 - protein, 107 aa, Artificial Sequence
  Description of Artificial Sequence Synthetic polypeptide
DIQMTQSPSSLSASVGDRVTITCRASQGISSWLAWYQQKPEKAPKSLIYAASSLQSGVPSRFSGSGSGTDFTLTISSLQPEDFATYYCQQYNSYPYTFGQGTKLEIK

SEQ ID NO: 206 - protein, 120 aa, Artificial Sequence
  Description of Artificial Sequence Synthetic polypeptide
QVQLVQSGAEVKKPGSSVKVSCKVSGGIFSTYAINWVRQAPGQGLEWMGGIIPIFGTANHAQKFQGRVTITADESTSTAYMELSSLRSEDTAVYYCARDQGIAAALFDYWGQGTLVTVSS

SEQ ID NO: 207 - protein, 108 aa, Artificial Sequence
  Description of Artificial Sequence Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 208 - protein, 113 aa, Artificial Sequence
  Description of Artificial Sequence Synthetic polypeptide
EVQLVESGGGLVQPGRSLRLSCAVSGFTFDDYVVHWVRQAPGKGLEWVSGISGNSGNIGYADSVKGRFTISRDNAKNSLYLQMNSLRAEDTALYYCAVPFDYWGQGTLVTVSS

SEQ ID NO: 209 - protein, 107 aa, Artificial Sequence; identical to SEQ ID NO: 205
  Description of Artificial Sequence Synthetic polypeptide

SEQ ID NO: 210 - protein, 123 aa, Artificial Sequence
  Description of Artificial Sequence Synthetic polypeptide
QVQLVQSGAEVKKPGSSVKVSCKTSGDTFSSYAISWVRQAPGQGLEWMGGIIPIFGRAHYAQKFQGRVTITADESTSTAYMELSSLRSEDTAVYFCARKFHFVSGSPFGMDVWGQGTTVTVSS

SEQ ID NO: 211 - protein, 106 aa, Artificial Sequence; identical to SEQ ID NO: 203
  Description of Artificial Sequence Synthetic polypeptide

SEQ ID NO: 212 - protein, 123 aa, Artificial Sequence
  Description of Artificial Sequence Synthetic polypeptide
QVQLVQSGAEVKKPGSSVKVSCKTSGGTFSSYAISWVRQAPGQGLEWMGGIIPIFGKAHYAQKFQGRVTITADESTTTAYMELSSLRSEDTAVYYCARKYDYVSGSPFGMDVWGQGTTVTVSS

SEQ ID NO: 213 - protein, 106 aa, Artificial Sequence; identical to SEQ ID NO: 203
  Description of Artificial Sequence Synthetic polypeptide

SEQ ID NO: 214 - protein, 121 aa, Artificial Sequence
  Description of Artificial Sequence Synthetic polypeptide
QVQLVQSGAEVKKPGSSVKVSCKASGGTFSSYAINWVRQAPGQGLEWMGGIIPIFGSANYAQKFQDRVTITADESTSAAYMELSSLRSEDTAVYYCARDSSGWSRYYMDVWGQGTTVTVSS

SEQ ID NO: 215 - protein, 106 aa, Artificial Sequence
  Description of Artificial Sequence Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPFGGGTKVEIK

SEQ ID NO: 216 - protein, 123 aa, Artificial Sequence
  Description of Artificial Sequence Synthetic polypeptide
QVQLVQSGAEVKEPGSSVKVSCKASGGTFNSYAISWVRQAPGQGLEWMGGIIPLFGIAHYAQKFQGRVTITADESTNTAYMDLSSLRSEDTAVYYCARKYSYVSGSPFGMDVWGQGTTVTVSS

SEQ ID NO: 217 - protein, 106 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTRLEIK

SEQ ID NO: 218 - protein, 121 aa, Artificial Sequence
  Description of Artificial Sequence Synthetic polypeptide
EVQLVESGGGLVQPGRSLRLSCAASGITFDDYGMHWVRQAPGKGLEWVSGISWNRGRIEYADSVKGRFTISRDNAKNSLYLQMNSLRAEDTALYYCAKGRFRYFDWFLDYWGQGTLVTVSS

SEQ ID NO: 219 - protein, 107 aa, Artificial Sequence
  Description of Artificial Sequence Synthetic polypeptide
AIQLTQSPSSLSASVGDRVTITCRASQGISSALAWYQQKPGKAPKLLIYDASSLESGVPSRFSGSGSGTDFTLTISSLQPEDFATYYCQQFNSYPFTFGPGTKVDIK

SEQ ID NO: 220 - protein, 121 aa, Artificial Sequence
  Description of Artificial Sequence Synthetic polypeptide
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 221 - protein, 108 aa, Artificial Sequence
  Description of Artificial Sequence Synthetic polypeptide
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 222 - protein, 120 aa, Artificial Sequence
  Description of Artificial Sequence Synthetic polypeptide
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

SEQ ID NO: 223 - protein, 110 aa, Artificial Sequence
  Description of Artificial Sequence Synthetic polypeptide
QSALTQPASVSGSPGQSITISCTGTSSDVGAYNYVSWYQQHPGKAPKLMIYDVSNRPSGVSNRFSGSKSGNTASLTISGLQAEDEADYYCSSYTSSSTRVFGTGTKVTVL

SEQ ID NO: 224 - protein, 117 aa, Artificial Sequence
  Description of Artificial Sequence Synthetic polypeptide
QVQLKESGPGLVAPSQSLSITCTVSGFSLTSYDISWVRQPPGKGLEWLGVIWTGVGTNYNSAFMSRLSISKDNSKSQVFLKMNSLQTDDTAMYYCVRDPYYYGMDYWGQGTSVTVSS

SEQ ID NO: 225 - protein, 107 aa, Artificial Sequence
  Description of Artificial Sequence Synthetic polypeptide
DIQMNQSPSSLSASLGDTITITCRASQDISIWLSWYQQKPGNIPELLIYKASNLHTGVPPRFSGSGSGTDFTLTISSLQPEDIATYYCLQSQSFPRTFGGGTKLEIK

SEQ ID NO: 226 - protein, 50 aa, Artificial Sequence
  Description of Artificial Sequence Synthetic polypeptide
  MISC_FEATURE (1)..(50): This sequence may encompass 1-10 “Gly Gly Gly Gly Ser” repeating units
  See specification as filed for detailed description of substitutions and preferred embodiments
GGGGSGGGGSGGGGSGGGGSGGGGSGGGGSGGGGSGGGGSGGGGSGGGGS

(19) United States
(12) Patent Application Publication
(10) Pub. No.: US 2023/0203119 A1
(43) Pub. Date: Jun. 29, 2023

(54) IMMUNOSTIMULATORY MULTIMERIC BINDING MOLECULES

(71) Applicant: IGM Biosciences, Inc., Mountain View, CA (US)

(72) Inventors: Ramesh BALIGA, Redwood City, CA (US); Thierry GIFFON, Sunnyvale, CA (US); Dean NG, San Francisco, CA (US)

(21) Appl. No.: 17/635,078

(22) PCT Filed: Aug. 14, 2020
(86) PCT No.: PCT/US2020/046379

§ 371 Date: Feb. 14, 2022

Related U.S. Application Data
(60) Provisional application No. 62/887,458, filed on Aug. 15, 2019.

(51) Int. Cl. C07K 14/54; C07K 14/715; C07K 14/55; C07K 16/28; A61P 35/00

(52) U.S. Cl. CPC C07K 14/5443; C07K 14/7155; C07K 14/55; C07K 16/2809; C07K 16/2827; C07K 16/2878; C07K 16/2887; A61P 35/00; A61K 2039/505

(57) ABSTRACT

This disclosure provides multivalent binding molecule comprising a modified J-chain that comprises an immune stimulatory agent. Also provided are polynucleotides encoding the binding molecule or subunits thereof and vectors and host cell comprising said polynucleotides. This disclosure further provides methods for producing and/or using a multivalent binding molecule comprising a modified J-chain that comprises an immune stimulatory agent.

### CROSS-REFERENCE TO RELATED APPLICATIONS

This application claims the benefit of U.S. Provisional Patent Application Ser. No. 62/887,458, filed Aug. 15, 2019, which is incorporated herein by reference in its entirety.

### SEQUENCE LISTING

The instant application contains a Sequence Listing which has been submitted electronically in ASCII format and is hereby incorporated by reference in its entirety. The ASCII copy was created on Aug. 13, 2020, is named 022W01-Sequence-Listing, and is 358,808 bytes in size.

### BACKGROUND

Antibodies and antibody-like molecules that can multimerize, such as IgA and IgM antibodies, have emerged as promising drug candidates in the fields of, e.g., immuno-oncology and infectious diseases allowing for improved specificity, improved avidity, and the ability to bind to multiple binding targets. See, e.g., U.S. Pat. Nos. 9,951,134 and 9,938,347, and PCT Publication Nos. WO 2016/141303, WO 2016/154593, WO 2016/168758, WO 2017/059387, WO 2017 059380, WO 2018/017888, WO 2018/017763, WO 2018/017889, WO 2018/017761, WO 2018/187702, and WO 2019/169314A1, the contents of which are incorporated herein by reference in their entireties.

Binding molecules, e.g., multimeric antibodies and antibody-like molecules, can be engineered to include additional moieties to enhance the immune response to difficult targets such as cancer cells, e.g., by stimulating effector immune cells, e.g., CD8+ T cells or NK cells. Independent immunotherapy using cytokines, e.g., IFN-α, IL-2, IL-12, IL-15, IL-21, or GM-CSF has been shown to be efficacious to some extent in the treatment of cancer and infection, but clinical outcome is often limited by toxicity associated with the high blood concentrations of untargeted cytokines that is needed to obtain efficacy.

IL-15 functions in modulating the activity of both the innate and adaptive immune system, e.g., maintenance of the memory T-cell response to invading pathogens, inhibition of apoptosis, activation of dendritic cells, and induction of Natural Killer (NK) cell proliferation and cytotoxic activity. See, e.g., PCT Publication No. WO 2018/134784 which is incorporated by reference in its entirety. Mature human IL-15 (SEQ ID NO: 4, amino acids 23 to 136 of GenBank Accession NO. CAA71044.1) shares approximately 96% amino acid sequence identity with mature cynomolgus IL-15 (amino acids 48-161 of SEQ ID NO: 71, GenBank EHH53989.1). Mature human and mouse IL-15 (amino acids 49-162 of SEQ ID NO: 72, SwissProt No. sp|P48346.1) have about 70% amino acid sequence identity.

The IL-15 receptor consists of three polypeptides, the type-specific IL-15 receptor alpha (“IL-15Rα”), the IL-2/IL-15 receptor beta (or CD122) (“β”), and the common gamma chain (or CD132) (“γ”) that is shared by multiple cytokine receptors. See, e.g., Anderson, D. M., et al., J Biol. Chem. 270: 29862-29869 (1995). IL-15Rα is thought to be expressed by a wide variety of cell types but not necessarily the same cells that express β and γ. See, e.g., PCT Publication No. WO 2018/134784. IL-15 can form a complex with IL-15 receptor alpha expressed on APCs prior to binding to functional IL-15 receptor β and γ subunits units on T cells or NK cells. Id. The IL-15Rα sushi domain is the critical component of IL-15Rα to form a complex with IL-15 prior to engagement with the β and γ receptor subunits (see, e.g., Wei et al. J. Immunol. 167:277-82 (2001)). The human sushi domain sequence presented as SEQ ID NO: 5, amino acids 31-107 of GenBank Accession No. NP_002180.1. The cynomolgus monkey sushi domain sequence is presented as amino acids 4-80 of SEQ ID NO: 73, GenBank Accession No. ACI42785.1 (92% amino acid identity to human sequence). The mouse sushi domain sequence is presented as amino acids 34-98 of SEQ ID NO: 74, SwissProt Accession No. sp|Q60819.1 (approximately 83% identity to human sequence). Sushi domain/IL-15 fusion proteins are reported to be highly potent at stimulating CD8+ T cells and NK cells compared to IL-15 alone (See, e.g., Mortier et al. J Biol Chem. 281:1612-19 (2005), and Stoklasek et al. J. Immunol. 177:6072-80 (2006)).

The antibody J-chain is an acidic 15-kDa polypeptide, which is associated with pentameric IgM and dimeric IgA via disulfide bonds involving the penultimate cysteine residue in the 18-amino acid secretory tailpiece (tp) at the C-terminus of the IgM μ or IgA α heavy chain. The precursor human J-chain amino acid sequence is presented as SEQ ID NO: 1, and the mature human J-chain amino acid sequence is presented as SEQ ID NO: 2. The assembly of IgM binding units into a pentameric structure is thought to involve the Cμ4 and tailpiece domains of the IgM constant region. See, e.g., Braathen, R., et al., J. Biol. Chem. 277:42755-42762 (2002).

Despite the advances made in the design of multimeric antibodies, there remains a need to be improve immunotherapy by engineering multimeric binding molecules.

### SUMMARY

This disclosure provides a multimeric binding molecule that includes two or five bivalent binding units or multimerizing variants or fragments thereof and a modified J-chain, where each binding unit includes two IgA or IgM heavy chain constant regions or multimerizing variants or fragments thereof, each associated with an antigen-binding domain for a total of four or ten antigen-binding domains, where at least three of the antigen-binding domains of the binding molecule specifically bind to a target antigen. As provided, the modified J-chain includes (a) a J-chain or functional fragment or variant thereof (“J”), and (b) an immunostimulatory agent (“ISA”), where J and the ISA are associated as a fusion protein.

In certain embodiments, the ISA includes a cytokine or receptor-binding fragment or variant thereof. For example, the cytokine or fragment or variant thereof includes IL-15 or IL-2, or a receptor-binding fragment or variant thereof. In certain embodiments, the ISA includes (a) an interleukin-15 (IL-15) protein or receptor-binding fragment or variant thereof (“I”), and (b) an interleukin-15 receptor-α (IL-15Rα) fragment including the sushi domain or a variant thereof capable of associating with I (“R”), where J and at least one of I and R are associated as a fusion protein, and where I and R can associate to function as the ISA.

In certain embodiments, J is a wild-type human J-chain and includes the amino acid sequence SEQ ID NO: 2 or a functional fragment or variant thereof. In other embodiments, J is a variant J-chain or fragment thereof including one or more single amino acid substitutions, deletions, or insertions relative to a wild-type J-chain that can affect serum half-life of the multimeric binding molecule; such that the multimeric binding molecule exhibits an increased serum half-life upon administration to an animal relative to a reference multimeric binding molecule that is identical except for the one or more single amino acid substitutions, deletions, or insertions, and is administered in the same way to the same animal species. For example, J can include an amino acid substitution at the amino acid position corresponding to amino acid Y102 of the mature wild-type human J-chain (SEQ ID NO: 2). The amino acid corresponding to Y102 of SEQ ID NO: 2 can be substituted with alanine (A), serine (S), or arginine (R), and in particular embodiments Y102 can be substituted with alanine (A). In certain embodiments J is a variant of the human J-chain and includes the amino acid sequence SEQ ID NO: 3 (“J*”) or amino acids 1-137 of SEQ ID NO: 86.

In some embodiments, J is a variant J-chain or fragment thereof comprising one or more single amino acid substitutions, deletions, or insertions relative to a wild-type J-chain that reduces glycosylation of the J. In some embodiments, the J comprises an amino acid substitution at the amino acid position corresponding to amino acid N49 of the mature wild-type human J-chain (SEQ ID NO: 2). In some embodiments, the amino acid corresponding to Y102 of SEQ ID NO: 2 is substituted with aspartic acid (D).

In certain embodiments, I includes the mature human IL-15 amino acid sequence of SEQ ID NO: 4 or a receptor-binding variant or fragment thereof. A receptor-binding variant can include at least one, but no more than ten, single amino acid insertions, deletions, or substitutions. In certain embodiments, the single amino acid insertions, deletions, or substitutions reduce, but do not eliminate, the affinity of the IL-15 variant for its receptor. In certain embodiments the variant I can include one, two, three, four, five, six, seven, or eight amino acid substitutions. In certain embodiments, the amino acid substitutions can be at one or more of positions corresponding to N1, N4, D8, D30, D61, E64, N65, N72, or Q108 of SEQ ID NO: 4. For example, the amino acid substitutions can include one or more of substitutions N1D, N4D, D8N, D30N, D61N, E64Q, N65D, N72D, or Q108E, in SEQ ID NO: 4. In certain embodiments, I includes SEQ ID NO: 4 except for: (a) a single amino acid substitution at a position selected from the group consisting of N1D, N4D, D8N, D30N, D61N, E64Q, N65D, N72D, and Q108E; (b) two amino acid substitutions at positions selected from the group consisting of N4D/N65D and N1D/N65D; or (c) three amino acid substitutions at positions D30N/E64Q/N65D. In certain embodiments, I includes the amino acid sequence SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, or SEQ ID NO: 68.

In some embodiments, the receptor-binding variant comprises at least one, but no more than ten, single amino acid insertions, deletions, or substitutions, and wherein the single amino acid insertions, deletions, or substitutions reduce the glycosylation of the IL-15 variant. In some embodiments, I comprises one, two, three, four, five, six, seven, or eight amino acid substitutions. In some embodiments, the amino acid substitutions are at one or more of positions corresponding to N71, S73, N79, or N112 of SEQ ID NO: 4. In some embodiments, the amino acid substitutions comprise one or more of substitutions N71D, S73I, N79D, or N112D in SEQ ID NO: 4. In some embodiments, I comprises SEQ ID NO: 4 except for one or more amino acid substitutions at a position selected from the group consisting of N71D, S73I, N79D, and N112D. In some embodiments, I comprises the amino acid sequence of amino acids 246-361 of SEQ ID NO: 85, SEQ ID NO: 87, SEQ ID NO: 88, SEQ ID NO: 89, or SEQ ID NO: 90.

In certain embodiments, R, the sushi domain of the IL-15 receptor-α, includes the amino acid sequence SEQ ID NO: 5 or a variant or fragment thereof that is capable of associating with human IL-15. In other embodiments, R consists essentially of or consists of the amino acid sequence SEQ ID NO: 5 or a variant thereof that is capable of associating with human IL-15.

In certain embodiments, J and I are associated as a fusion protein. In certain embodiments, J and R are associated as a fusion protein. In certain embodiments, J, I, and R are associated as a fusion protein. According to these embodiments, J, I, and/or R can be fused via linkers, which can be the same or different. In certain embodiments, at least one linker includes, consists essentially of, or consists of the amino acid sequence GGGGSGGGGSGGGGS (SEQ ID NO: 78). In certain embodiments, at least one linker includes, consists essentially of, or consists of the amino acid sequence GGSGGGGSGGGSGGGGSLQ (SEQ ID NO: 79). In certain embodiments, J is J*, and the modified J-chain can be arranged from N-terminus to C-terminus as J*-R-I, J*-I-R, I-R-J*, R-I-J*, R-J*-I, I-J*-R, I-J*, or J*-I, where “-” is a linker. In certain embodiments, the modified J-chain includes the amino acid sequence SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 77, SEQ ID NO: 85, SEQ ID NO: 86, SEQ ID NO: 87, SEQ ID NO: 88, SEQ ID NO: 89, or SEQ ID NO: 90. In a particular embodiment, the modified J-chain is arranged from N-terminus to C-terminus as J*-R-I, and can include the amino acid sequence SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 77, SEQ ID NO: 85, SEQ ID NO: 86, SEQ ID NO: 87, SEQ ID NO: 88, SEQ ID NO: 89, or SEQ ID NO: 90.

In other embodiments, the ISA includes a variant of human IL-2, for example, “IL2v,” that does not bind to the α-subunit of the IL-2 receptor. IL2v includes the amino acid sequence SEQ ID NO: 31, and one modified J-chain comprising IL2v includes the amino acid sequence SEQ ID NO: 32.

In certain embodiments, the modified J-chain of a multimeric binding molecule as provided herein can further include an antigen-binding domain of an antibody fused thereto, in addition to the ISA. For example, the modified J-chain can include an antigen-binding domain binds to a target on an immune effector cell. In certain embodiments, the immune effector cell is a CD8+ T cell, and the antigen binding domain can be a single-chain Fv (scFv) antibody fragment that specifically binds to CD3epsilon (CD3ε). According to these embodiments, the modified J-chain can include the amino acid sequence SEQ ID NO: 19.

In certain embodiments, the multimeric binding molecule provided by this disclosure is pentameric and includes five binding units, where each binding unit includes two IgM heavy chain constant regions or multimerizing variants or fragments thereof. In other embodiments, the multimeric binding molecule provided by this disclosure is dimeric and includes two binding units or multimerizing variants or fragments thereof, where each binding unit includes two IgA heavy chain constant regions or multimerizing variants or fragments thereof.

In certain embodiments, the target antigen bound by the multimeric binding molecule provided by this disclosure is a tumor-associated antigen or a target that modulates a T cell response or NK cell response. For example, the target antigen can include a target that modulates a T cell response or an NK cell response. In certain embodiments, the target is one that inhibits CD8+ T cell or NK cell activity and it is desirable to inhibit such a target. For example, that target can be an inhibitory immune checkpoint protein, and the antigen-binding domains of the provided binding molecule antagonize the target, thereby stimulating CD8+ T cells or NK cells. For example, the inhibitory immune checkpoint protein includes a programmed cell death-1 protein (PD-1), a programmed cell death ligand-1 protein (PD-L1), a lymphocyte-activation gene 3 protein (LAG3), a T-cell immunoglobulin and mucin domain 3 protein (TIM3), a cytotoxic T-lymphocyte-associated protein 4 (CTLA4), a B- and T-lymphocyte attenuator protein (BTLA), a V-domain Ig suppressor of T-cell activation protein (VISTA), a T-cell immunoreceptor with Ig and ITIM Domains protein (TIGIT), a Killer-cell Immunoglobulin-like Receptor protein (KIR), a B7-H3 protein, a B7-H4 protein, or any combination thereof. In a particular embodiment, the inhibitory immune checkpoint protein is PD-L1. According to this embodiment, the antigen-binding domain can include a heavy chain variable region (VH) that includes the amino acid sequence SEQ ID NO: 33, SEQ ID NO: 91, SEQ ID NO: 92, or SEQ ID NO: 93 and a light chain variable region (VL) that includes the amino acid sequence SEQ ID NO: 34 or SEQ ID NO: 94.

In some embodiments, the inhibitory immune checkpoint protein comprises PD-L1, and the antigen-binding domain comprises a heavy chain variable region (VH) and a light chain variable region (VL) wherein the VH and VL comprise six immunoglobulin complementarity determining regions HCDR1, HCDR2, HCDR3, LCDR1, LCDR2, and LCDR3, wherein the HCDR1, HCDR2, HCDR3, LCDR1, LCDR2, and LCDR3 comprise the CDRs of an antibody comprising the VH and VL of SEQ ID NO: 75 and SEQ ID NO: 76, SEQ ID NO: 96 and SEQ ID NO: 97, SEQ ID NO: 98 and SEQ ID NO: 99, SEQ ID NO: 100 and SEQ ID NO: 101, SEQ ID NO: 102 and SEQ ID NO: 103, SEQ ID NO: 104 and SEQ ID NO: 105, SEQ ID NO: 106 and SEQ ID NO: 107, SEQ ID NO: 108 and SEQ ID NO: 109, SEQ ID NO: 110 and SEQ ID NO: 111, SEQ ID NO: 112 and SEQ ID NO: 113, SEQ ID NO: 114 and SEQ ID NO: 115, SEQ ID NO: 116 and SEQ ID NO: 117, SEQ ID NO: 118 and SEQ ID NO: 119, SEQ ID NO: 120 and SEQ ID NO: 121, SEQ ID NO: 122 and SEQ ID NO: 123, SEQ ID NO: 124 and SEQ ID NO: 125, SEQ ID NO: 126 and SEQ ID NO: 127, SEQ ID NO: 128 and SEQ ID NO: 129, SEQ ID NO: 130 and SEQ ID NO: 131, SEQ ID NO: 132 and SEQ ID NO: 133, SEQ ID NO: 134 and SEQ ID NO: 135, SEQ ID NO: 136 and SEQ ID NO: 137, SEQ ID NO: 138 and SEQ ID NO: 139, SEQ ID NO: 140 and SEQ ID NO: 141, SEQ ID NO: 142 and SEQ ID NO: 143, SEQ ID NO: 144 and SEQ ID NO: 145, SEQ ID NO: 146 and SEQ ID NO: 147, SEQ ID NO: 148 and SEQ ID NO: 149, SEQ ID NO: 150 and SEQ ID NO: 151, SEQ ID NO: 152 and SEQ ID NO: 153, SEQ ID NO: 154 and SEQ ID NO: 155, SEQ ID NO: 156 and SEQ ID NO: 157, SEQ ID NO: 158 and SEQ ID NO: 159, SEQ ID NO: 160 and SEQ ID NO: 161, SEQ ID NO: 162 and SEQ ID NO: 163, SEQ ID NO: 164 and SEQ ID NO: 165, SEQ ID NO: 166 and SEQ ID NO: 167, SEQ ID NO: 168 and SEQ ID NO: 169, SEQ ID NO: 170 and SEQ ID NO: 171, SEQ ID NO: 172 and SEQ ID NO: 173, SEQ ID NO: 174 and SEQ ID NO: 175, SEQ ID NO: 176 and SEQ ID NO: 177, SEQ ID NO: 178 and SEQ ID NO: 179, SEQ ID NO: 180 and SEQ ID NO: 181, SEQ ID NO: 182 and SEQ ID NO: 183, SEQ ID NO: 184 and SEQ ID NO: 185, SEQ ID NO: 186 and SEQ ID NO: 187, SEQ ID NO: 188 and SEQ ID NO: 189, SEQ ID NO: 190 and SEQ ID NO: 191, SEQ ID NO: 192 and SEQ ID NO: 193, SEQ ID NO: 194 and SEQ ID NO: 195, SEQ ID NO: 196 and SEQ ID NO: 197, SEQ ID NO: 198 and SEQ ID NO: 199, SEQ ID NO: 200 and SEQ ID NO: 201, SEQ ID NO: 202 and SEQ ID NO: 203, SEQ ID NO: 204 and SEQ ID NO: 205, SEQ ID NO: 206 and SEQ ID NO: 207, SEQ ID NO: 208 and SEQ ID NO: 209, SEQ ID NO: 210 and SEQ ID NO: 211, SEQ ID NO: 212 and SEQ ID NO: 213, SEQ ID NO: 214 and SEQ ID NO: 215, SEQ ID NO: 216 and SEQ ID NO: 217, SEQ ID NO: 218 and SEQ ID NO: 219, SEQ ID NO: 220 and SEQ ID NO: 221, or SEQ ID NO: 222 and SEQ ID NO: 223, respectively with zero, one, or two single amino acid substitutions in one or more of the HCDRs or LCDRs. In some embodiments, the inhibitory immune checkpoint protein comprises PD-L1, and the antigen-binding domain comprises a heavy chain variable region (VH) and a light chain variable region (VL) wherein the VH and VL comprise six immunoglobulin complementarity determining regions HCDR1, HCDR2, HCDR3, LCDR1, LCDR2, and LCDR3, wherein the HCDR1, HCDR2, HCDR3, LCDR1, LCDR2, and LCDR3 comprise the CDRs of an antibody comprising the VH and VL of SEQ ID NO: 134 and SEQ ID NO: 135, SEQ ID NO: 136 and SEQ ID NO: 137, SEQ ID NO: 138 and SEQ ID NO: 139, SEQ ID NO: 140 and SEQ ID NO: 141, SEQ ID NO: 142 and SEQ ID NO: 143, SEQ ID NO: 144 and SEQ ID NO: 145, SEQ ID NO: 146 and SEQ ID NO: 147, SEQ ID NO: 148 and SEQ ID NO: 149, SEQ ID NO: 166 and SEQ ID NO: 167, SEQ ID NO: 168 and SEQ ID NO: 169, SEQ ID NO: 170 and SEQ ID NO: 171, or SEQ ID NO: 186 and SEQ ID NO: 187, respectively with zero, one, or two single amino acid substitutions in one or more of the HCDRs or LCDRs, such as zero amino acid substitutions.

In some embodiments, the inhibitory immune checkpoint protein comprises PD-L1, and the antigen-binding domain comprises a heavy chain variable region (VH) and a light chain variable region (VL) wherein the VH and VL comprise amino acid sequences at least 80%, at least 85%, at least 90%, at least 95% or 100% identical to the mature VH and VL amino acid sequences comprising SEQ ID NO: 75 and SEQ ID NO: 76, SEQ ID NO: 96 and SEQ ID NO: 97, SEQ ID NO: 98 and SEQ ID NO: 99, SEQ ID NO: 100 and SEQ ID NO: 101, SEQ ID NO: 102 and SEQ ID NO: 103, SEQ ID NO: 104 and SEQ ID NO: 105, SEQ ID NO: 106 and SEQ ID NO: 107, SEQ ID NO: 108 and SEQ ID NO: 109, SEQ ID NO: 110 and SEQ ID NO: 111, SEQ ID NO: 112 and SEQ ID NO: 113, SEQ ID NO: 114 and SEQ ID NO: 115, SEQ ID NO: 116 and SEQ ID NO: 117, SEQ ID NO: 118 and SEQ ID NO: 119, SEQ ID NO: 120 and SEQ ID NO: 121, SEQ ID NO: 122 and SEQ ID NO: 123, SEQ ID NO: 124 and SEQ ID NO: 125, SEQ ID NO: 126 and SEQ ID NO: 127, SEQ ID NO: 128 and SEQ ID NO: 129, SEQ ID NO: 130 and SEQ ID NO: 131, SEQ ID NO: 132 and SEQ ID NO: 133, SEQ ID NO: 134 and SEQ ID NO: 135, SEQ ID NO: 136 and SEQ ID NO: 137, SEQ ID NO: 138 and SEQ ID NO: 139, SEQ ID NO: 140 and SEQ ID NO: 141, SEQ ID NO: 142 and SEQ ID NO: 143, SEQ ID NO: 144 and SEQ ID NO: 145, SEQ ID NO: 146 and SEQ ID NO: 147, SEQ ID NO: 148 and SEQ ID NO: 149, SEQ ID NO: 150 and SEQ ID NO: 151, SEQ ID NO: 152 and SEQ ID NO: 153, SEQ ID NO: 154 and SEQ ID NO: 155, SEQ ID NO: 156 and SEQ ID NO: 157, SEQ ID NO: 158 and SEQ ID NO: 159, SEQ ID NO: 160 and SEQ ID NO: 161, SEQ ID NO: 162 and SEQ ID NO: 163, SEQ ID NO: 164 and SEQ ID NO: 165, SEQ ID NO: 166 and SEQ ID NO: 167, SEQ ID NO: 168 and SEQ ID NO: 169, SEQ ID NO: 170 and SEQ ID NO: 171, SEQ ID NO: 172 and SEQ ID NO: 173, SEQ ID NO: 174 and SEQ ID NO: 175, SEQ ID NO: 176 and SEQ ID NO: 177, SEQ ID NO: 178 and SEQ ID NO: 179, SEQ ID NO: 180 and SEQ ID NO: 181, SEQ ID NO: 182 and SEQ ID NO: 183, SEQ ID NO: 184 and SEQ ID NO: 185, SEQ ID NO: 186 and SEQ ID NO: 187, SEQ ID NO: 188 and SEQ ID NO: 189, SEQ ID NO: 190 and SEQ ID NO: 191, SEQ ID NO: 192 and SEQ ID NO: 193, SEQ ID NO: 194 and SEQ ID NO: 195, SEQ ID NO: 196 and SEQ ID NO: 197, SEQ ID NO: 198 and SEQ ID NO: 199, SEQ ID NO: 200 and SEQ ID NO: 201, SEQ ID NO: 202 and SEQ ID NO: 203, SEQ ID NO: 204 and SEQ ID NO: 205, SEQ ID NO: 206 and SEQ ID NO: 207, SEQ ID NO: 208 and SEQ ID NO: 209, SEQ ID NO: 210 and SEQ ID NO: 211, SEQ ID NO: 212 and SEQ ID NO: 213, SEQ ID NO: 214 and SEQ ID NO: 215, SEQ ID NO: 216 and SEQ ID NO: 217, SEQ ID NO: 218 and SEQ ID NO: 219, SEQ ID NO: 220 and SEQ ID NO: 221, or SEQ ID NO: 222 and SEQ ID NO: 223, respectively. In some embodiments, the inhibitory immune checkpoint protein comprises PD-L1, and the antigen-binding domain comprises a heavy chain variable region (VH) and a light chain variable region (VL) wherein the VH and VL comprise amino acid sequences at least 80%, at least 85%, at least 90%, at least 95% or 100% identical to the mature VH and VL amino acid sequences comprising SEQ ID NO: 134 and SEQ ID NO: 135, SEQ ID NO: 136 and SEQ ID NO: 137, SEQ ID NO: 138 and SEQ ID NO: 139, SEQ ID NO: 140 and SEQ ID NO: 141, SEQ ID NO: 142 and SEQ ID NO: 143, SEQ ID NO: 144 and SEQ ID NO: 145, SEQ ID NO: 146 and SEQ ID NO: 147, SEQ ID NO: 148 and SEQ ID NO: 149, SEQ ID NO: 166 and SEQ ID NO: 167, SEQ ID NO: 168 and SEQ ID NO: 169, SEQ ID NO: 170 and SEQ ID NO: 171, or SEQ ID NO: 186 and SEQ ID NO: 187, respectively.

In other embodiments, the target antigen bound by the multimeric binding molecule provided by this disclosure is a TNF receptor superfamily target. According to these embodiments, the antigen-binding domains can agonize the target. For example, the target antigen can be GITR, OX40, or a combination thereof. In those embodiments where the target antigen is GITR, the antigen-binding domain can include, for example, a heavy chain variable region (VH) and a light chain variable region (VL) including, respectively, the amino acid sequences SEQ ID NO: 35 and SEQ ID NO: 36, SEQ ID NO: 37 and SEQ ID NO: 38, SEQ ID NO: 39 and SEQ ID NO: 40, SEQ ID NO: 41 and SEQ ID NO: 42, or SEQ ID NO: 43 and SEQ ID NO: 44. In those embodiments where the target antigen includes OX40, the antigen-binding domain can include, for example, a heavy chain variable region (VH) and a light chain variable region (VL) including, respectively, the amino acid sequences SEQ ID NO: 45 and SEQ ID NO: 46 or SEQ ID NO: 47 and SEQ ID NO: 48.

In other embodiments, the target antigen bound by the multimeric binding molecule provided by this disclosure is a tumor-associated antigen. The tumor-associated antigen can include, for example, B-cell maturation antigen (BCMA), CD19, CD20, EGFR, HER2 (ErbB2), ErbB3, ErbB4, CTLA4, PD-1, PD-L1, VEGF, VEGFR1, VEGFR2, CD52, CD30, prostate-specific membrane antigen (PSMA), CD38, GD2, SLAMF7, platelet-derived growth factor receptor A (PDGFRA), CD22, FLT3 (CD135), CD123, MUC-16, carcinoembryonic antigen-related cell adhesion molecule 1 (CEACAM-1), mesothelin, tumor-associated calcium signal transducer 2 (Trop-2), glypican-3 (GPC-3), human blood group H type 1 trisaccharide (Globo-H), sialyl Tn antigen (STn antigen), CD33, or any combination thereof. In a particular aspect, the target antigen is CD20, and the antigen-binding domain can include, for example, a heavy chain variable region (VH) and a light chain variable region (VL) including, respectively, the amino acid sequences SEQ ID NO: 49 and SEQ ID NO: 50.

In certain embodiments at least four, at least five, at least six, at least seven, at least eight, at least nine or ten of the antigen-binding domains of the binding molecule specifically bind to the same target antigen.

In certain embodiments, each binding unit of a multimeric binding molecule provided by the disclosure includes two IgA heavy chain constant regions or multimerizing fragments or variants thereof, each including an IgA Cal domain, an IgA hinge, an IgA Cα2 domain, IgA Cα3 domain, and an IgA tailpiece domain. In certain embodiments, the IgA heavy chain constant regions include the amino acid sequence SEQ ID NO: 53, SEQ ID NO: 54, or any multimerizing variant or fragment thereof.

In certain embodiments, each binding unit of a multimeric binding molecule provided by the disclosure includes two IgM heavy chain constant regions or multimerizing fragments or variants thereof, each including an IgM Cμ4 domain and an IgM tailpiece domain. In certain embodiments, each IgM heavy chain constant region or multimerizing fragment or variant thereof further includes an IgM Cμ3 domain, an IgM Cμ2 domain, an IgM Cμ1 domain, or any combination thereof, situated N-terminal to the IgM Cμ4 and IgM tailpiece domains. In certain embodiments, each IgM heavy chain constant region is a human IgM constant region or multimerizing variant or fragment thereof, including the amino acid sequence SEQ ID NO: 51, SEQ ID NO: 52, or a multimerizing variant or fragment thereof.

In certain embodiments, each IgM heavy chain constant region is a variant of a human IgM constant region or multimerizing fragment thereof, which confers reduced CDC activity to the multimeric binding molecule relative to a multimeric binding molecule including IgM heavy chain constant regions including the amino acid sequence SEQ ID NO: 51, SEQ ID NO: 52. For example, each IgM heavy chain constant region can include a variant of the amino acid sequence SEQ ID NO: 51 or SEQ ID NO: 52, where the variant includes an amino acid substitution at position P311 of SEQ ID NO: 51 or SEQ ID NO: 52, an amino acid substitution at position P313 of SEQ ID NO: 51 or SEQ ID NO: 52, or amino acid substitutions at positions P311 and P313 of SEQ ID NO: 51 or SEQ ID NO: 52.

In certain embodiments, each IgM heavy chain constant region is a variant of a human IgM constant region or multimerizing fragment thereof, which confers increased serum half-life to the multimeric binding molecule upon administration to a subject animal relative to a multimeric binding molecule including the reference IgM heavy chain constant regions, and is administered in the same way to the same animal species. For example, the variant IgM heavy chain constant regions can include amino acid substitutions at one or more amino acid positions corresponding to amino acid, E345A, S401A, E402A, or E403A of the wild-type human IgM constant region SEQ ID NO: 51 or SEQ ID NO: 52.

This disclosure also provides an isolated polynucleotide that includes a nucleic acid encoding a subunit polypeptide of the multimeric binding molecule provided by the disclosure, where the subunit polypeptide includes (a) an IgA or IgM heavy chain including an IgA or IgM heavy chain constant region or a multimerizing variant or fragment thereof associated with an antibody heavy chain variable region (VH), (b) an antibody light chain including an antibody light chain constant region associated with an antibody light chain variable region (VL), or (c) a modified J-chain including two or more of (i) a J-chain or functional fragment or variant thereof (“J”), (ii) an interleukin-15 (IL-15) protein or receptor-binding fragment or variant thereof (“I”), or (iii) an interleukin-15 receptor-α (IL-15Rα) fragment including the sushi domain or a variant thereof capable of associating with I (“R”), where J and at least one of I and R are associated as a fusion protein, and where I and R can associate to function as an immune stimulatory complex, or (d) any combination thereof.

Also provided is an expression vector that includes the polynucleotide and a host cell that includes the polynucleotide and/or the expression vector. The disclosure also provides a method for producing the provided multimeric binding molecule that includes culturing the host cell and recovering the multimeric binding molecule.

This disclosure further provides a method for treating cancer, that includes administering to a subject in need of treatment an effective amount of the provided multimeric binding molecule.

### BRIEF DESCRIPTION OF THE DRAWINGS/FIGURES

FIG. 1 shows an exemplary IgM pentamer with a modified human J-chain comprising a Y102A mutation (“J*”) fused to IL-15 (“I,” denoted as a circle) and the sushi domain of IL-15 receptor-α (“R,” denoted as an oval) in various orientations. The configurations from N-terminus to C-terminus are J*RI, IRJ*, RJ*I, J*IR, RIJ*, and IJ*R.

FIG. 2 is a schematic of the IL-15 Potency Assay with human peripheral blood mononuclear cells (PBMCs). The schematic shows the monocytes expressing PD-L1, but the assay is applicable to targeting other antigens expressed on monocytes or other peripheral blood mononuclear cells.

FIG. 3 shows the in vitro potency of various IL-15-IL-15 receptor-α (“RI”) ISA compounds as provided herein. The data show proliferation of CD8+ T cells as indicated by Ki67 positivity in response to increasing concentrations of the RI compounds. h3C5 IgM+JH is shown as a negative control.

FIG. 4 shows the effect of h3C5 IgM+J*RI on the proliferation of various T cell subsets as indicated by Ki67 positivity.

FIGS. 5A-5D show the effects of single, double, and triple mutations in IL-15 on the potency of 3C5 IgM+J*RI ISA constructs to trigger proliferation of CD8+ T cells or NK cells as indicated by Ki67 positivity. FIG. 5A shows the effect of single IL-15 mutations on CD8+ T cell proliferation; FIG. 5B shows the effect of single IL-15 mutations on NK cell proliferation; FIG. 5C shows the effect of double and triple IL-15 mutations on CD8+ T cell proliferation; and FIG. 5D shows the effect of double and triple IL-15 mutations on NK cell proliferation.

FIG. 6 shows that hu3C5+J*RI upregulates GITR and OX-40 expression on cytotoxic CD8+ T cells to a greater extent than other ISAs tested, including HRI, 153+J*RI, KD-RI, or hu3C5+JH (J-chain fused to human serum albumin). The data shown is for 5 nM of each compound.

FIG. 7 shows that three different anti-GITR IgM+J*RI constructs, GITR IgM_J*RI mab #23, GITR IgM_J*RI mab #14, and GITR IgM_J*RI mab #12, can trigger proliferation of CD8+ T cells.

FIGS. 8A-8D show the potency of h3C5 IgM+SJ*RI in a Ki-67 proliferation assay for CD8+ T cells (FIGS. 8A and 8B, two different PBMC donors) or for NK cells (FIGS. 8C and 8D, two different PBMC donors), where “S” is an scFv fragment of the anti-CD3 SP34 antibody.

FIGS. 9A-9D show the effect of m3c5-J*RI treatment in a hPD-L1-CT26 mouse efficacy model. FIG. 9A shows the average tumor size in control (Vehicle) and treatment (m3c5-J*RI and anti-PD-L1 IgG) groups; FIGS. 9B-9D show the individual tumor size in the vehicle group (FIG. 9B), anti-PD-L1 IgG group (FIG. 9C), and m3c5-J*RI group (FIG. 9D).

FIG. 10A-10D show the re-challenge with CT26 tumor cells of treated mice having rejected tumors and a naïve control group. FIG. 10A shows the average tumor size. FIG. 10B shows individual tumor size in the naïve and treatment groups at Day38. FIGS. 10C-10D show individual tumor size in the naïve and treatment groups.

FIGS. 11A-11D show the effects of m3c5-J*RI dose-dependent treatment in a BALB/c pharmacodynamic model. FIG. 11A shows the number of peripheral CD8 T cells after treatment. FIG. 11B shows the number of peripheral NK cells after treatment. FIG. 11C shows the number of peripheral CD4 T cells after treatment. FIG. 11D shows the number of peripheral B cells after treatment.

FIG. 12 shows the effects of various mutations of J*RI glycosylation sites on the proliferation of human CD8 T cells.

FIGS. 13A-13B show the effects of m3c5-J*RI on proliferation of human (FIG. 13A) and cynomolgus (FIG. 13B) CD8 T cells.

FIGS. 14A-14F show the lack of secretion of inflammatory cytokines human IL-6 (FIG. 14A), human IFNγ (FIG. 14B), human TNFα (FIG. 14C), cynomolgus IL-6 (FIG. 14D), cynomolgus IFNγ (FIG. 14E), or cynomolgus TNFα (FIG. 14F) elicited by m3c5-J*RI in an in vitro potency assay.

FIG. 15A-15C shows the effects of m3c5-J*RI on human PBMC, NK cells, and CD8+ T cells, respectively, in an MDA-MB-231 cell line in vitro killing assay.

FIG. 16 shows the potential epitope bound by the 3C5 Fab based on alanine scanning mutagenesis of PD-L1.

### DETAILED DESCRIPTION

### Definitions

It is to be noted that the term “a” or “an” entity refers to one or more of that entity; for example, “a binding molecule,” is understood to represent one or more binding molecules. As such, the terms “a” (or “an”), “one or more,” and “at least one” can be used interchangeably herein.

Furthermore, “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Unless defined otherwise, technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure.

Units, prefixes, and symbols are denoted in their Systéme International de Unites (SI) accepted form. Numeric ranges are inclusive of the numbers defining the range. Unless otherwise indicated, amino acid sequences are written left to right in amino to carboxy orientation. The headings provided herein are not limitations of the various aspects or aspects of the disclosure, which can be had by reference to the specification as a whole. Accordingly, the terms defined immediately below are more fully defined by reference to the specification in its entirety.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids and does not refer to a specific length of the product. Thus, peptides, dipeptides, tripeptides, oligopeptides, “protein,” “amino acid chain,” or any other term used to refer to a chain or chains of two or more amino acids are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, and derivatization by known protecting/blocking groups, proteolytic cleavage, or modification by non-naturally occurring amino acids. A polypeptide can be derived from a biological source or produced by recombinant technology but is not necessarily translated from a designated nucleic acid sequence. It can be generated in any manner, including by chemical synthesis.

A polypeptide as disclosed herein can be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides can have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations and are referred to as unfolded. As used herein, the term glycoprotein refers to a protein coupled to at least one carbohydrate moiety that is attached to the protein via an oxygen-containing or a nitrogen-containing side chain of an amino acid, e.g., a serine or an asparagine.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated as disclosed herein, as are native or recombinant polypeptides which have been separated, fractionated, or partially or substantially purified by any suitable technique.

As used herein, the term “a non-naturally occurring polypeptide” or any grammatical variants thereof, is a conditional definition that explicitly excludes, but only excludes, those forms of the polypeptide that are, or might be, determined or interpreted by a judge or an administrative or judicial body, to be “naturally-occurring.”

Other polypeptides disclosed herein are fragments, derivatives, analogs, or variants of the foregoing polypeptides, and any combination thereof. The terms “fragment,” “variant,” “derivative” and “analog” as disclosed herein include any polypeptides which retain at least some of the properties of the corresponding native antibody or polypeptide, for example, specifically binding to an antigen. Fragments of polypeptides include, for example, proteolytic fragments, as well as deletion fragments, in addition to specific antibody fragments discussed elsewhere herein. Variants of, e.g., a polypeptide include fragments as described above, and polypeptides with altered amino acid sequences due to amino acid substitutions, deletions, or insertions. In certain aspects, variants can be non-naturally occurring. Non-naturally occurring variants can be produced using art-known mutagenesis techniques. Variant polypeptides can comprise conservative or non-conservative amino acid substitutions, deletions or additions. Derivatives are polypeptides that have been altered so as to exhibit additional features not found on the original polypeptide. Examples include fusion proteins. Variant polypeptides can also be referred to herein as “polypeptide analogs.” As used herein a “derivative” of a polypeptide can also refer to a subject polypeptide having one or more amino acids chemically derivatized by reaction of a functional side group. Also included as “derivatives” are those peptides that contain one or more derivatives of the twenty standard amino acids. For example, 4-hydroxyproline can be substituted for proline; 5-hydroxylysine can be substituted for lysine; 3-methylhistidine can be substituted for histidine; homoserine can be substituted for serine; and ornithine can be substituted for lysine.

A “conservative amino acid substitution” is one in which one amino acid is replaced with another amino acid having a similar side chain. Families of amino acids having similar side chains have been defined in the art, including basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. In certain embodiments, conservative substitutions in the sequences of the polypeptides and antibodies of the present disclosure do not abrogate the binding of the polypeptide or antibody containing the amino acid sequence, to the antigen to which the antibody binds. Methods of identifying nucleotide and amino acid conservative substitutions which do not eliminate antigen-binding are well-known in the art (see, e.g., Brummell et al., Biochem. 32: 1180-1 187 (1993); Kobayashi et al., Protein Eng. 12(10):879-884 (1999); and Burks et al., Proc. Natl. Acad. Sci. USA 94:412-417 (1997)).

The term “polynucleotide” is intended to encompass a singular nucleic acid as well as plural nucleic acids and refers to an isolated nucleic acid molecule or construct, e.g., messenger RNA (mRNA), cDNA, or plasmid DNA (pDNA). A polynucleotide can comprise a conventional phosphodiester bond or a non-conventional bond (e.g., an amide bond, such as found in peptide nucleic acids (PNA)). The terms “nucleic acid” or “nucleic acid sequence” refer to any one or more nucleic acid segments, e.g., DNA or RNA fragments, present in a polynucleotide.

By an “isolated” nucleic acid or polynucleotide is intended any form of the nucleic acid or polynucleotide that is separated from its native environment. For example, gel-purified polynucleotide, or a recombinant polynucleotide encoding a polypeptide contained in a vector would be considered to be “isolated.” Also, a polynucleotide segment, e.g., a PCR product, which has been engineered to have restriction sites for cloning is considered to be “isolated.” Further examples of an isolated polynucleotide include recombinant polynucleotides maintained in heterologous host cells or purified (partially or substantially) polynucleotides in a non-native solution such as a buffer or saline. Isolated RNA molecules include in vivo or in vitro RNA transcripts of polynucleotides, where the transcript is not one that would be found in nature. Isolated polynucleotides or nucleic acids further include such molecules produced synthetically. In addition, polynucleotide or a nucleic acid can be or can include a regulatory element such as a promoter, ribosome binding site, or a transcription terminator.

As used herein, the term “a non-naturally occurring polynucleotide” or any grammatical variants thereof, is a conditional definition that explicitly excludes, but only excludes, those forms of the nucleic acid or polynucleotide that are, or might be, determined or interpreted by a judge, or an administrative or judicial body, to be “naturally-occurring.”

As used herein, a “coding region” is a portion of nucleic acid which consists of codons translated into amino acids. Although a “stop codon” (TAG, TGA, or TAA) is not translated into an amino acid, it can be considered to be part of a coding region, but any flanking sequences, for example promoters, ribosome binding sites, transcriptional terminators, introns, and the like, are not part of a coding region. Two or more coding regions can be present in a single polynucleotide construct, e.g., on a single vector, or in separate polynucleotide constructs, e.g., on separate (different) vectors. Furthermore, any vector can contain a single coding region, or can comprise two or more coding regions, e.g., a single vector can separately encode an immunoglobulin heavy chain variable region and an immunoglobulin light chain variable region. In addition, a vector, polynucleotide, or nucleic acid can include heterologous coding regions, either fused or unfused to another coding region. Heterologous coding regions include without limitation, those encoding specialized elements or motifs, such as a secretory signal peptide or a heterologous functional domain.

In certain embodiments, the polynucleotide or nucleic acid is DNA. In the case of DNA, a polynucleotide comprising a nucleic acid which encodes a polypeptide normally can include a promoter and/or other transcription or translation control elements operably associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter can be a cell-specific promoter that directs substantial transcription of the DNA in predetermined cells. Other transcription control elements, besides a promoter, for example enhancers, operators, repressors, and transcription termination signals, can be operably associated with the polynucleotide to direct cell-specific transcription.

A variety of transcription control regions are known to those skilled in the art. These include, without limitation, transcription control regions which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

In other embodiments, a polynucleotide can be RNA, for example, in the form of messenger RNA (mRNA), transfer RNA, or ribosomal RNA.

Polynucleotide and nucleic acid coding regions can be associated with additional coding regions which encode secretory or signal peptides, which direct the secretion of a polypeptide encoded by a polynucleotide as disclosed herein. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells can have a signal peptide fused to the N-terminus of the polypeptide, which is cleaved from the complete or “full length” polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, can be used. For example, the wild-type leader sequence can be substituted with the leader sequence of human tissue plasminogen activator (TPA) or mouse β-glucuronidase.

As used herein, the term “binding molecule” refers in its broadest sense to a molecule that specifically binds to a binding target, e.g., an epitope or an antigenic determinant. As described further herein, a binding molecule can comprise one of more “antigen-binding domains” described herein. A non-limiting example of a binding molecule is an antibody or antibody-like molecule as described in detail herein that retains antigen-specific binding. In certain aspects a “binding molecule” comprises an antibody or antibody-like molecule as described in detail herein.

As used herein, the terms “binding domain” or “antigen-binding domain” (can be used interchangeably) refer to a region of a binding molecule, e.g., an antibody or antibody-like molecule, that is necessary and sufficient to specifically bind to a binding target, e.g., an epitope. For example, an “Fv,” e.g., a heavy chain variable region and a light chain variable region of an antibody, either as two separate polypeptide subunits or as a single chain, is considered to be a “binding domain.” Other antigen-binding domains include, without limitation, the heavy chain variable region (VHH) of an antibody derived from a camelid species, or six immunoglobulin complementarity determining regions (CDRs) expressed in a scaffold, e.g., a fibronectin scaffold. A “binding molecule,” or “antibody” as described herein can include one, two, three, four, five, six, seven, eight, nine, ten, eleven, twelve, or even more “antigen-binding domains.”

The terms “antibody” and “immunoglobulin” can be used interchangeably herein. An antibody (or a fragment, variant, or derivative thereof as disclosed herein) includes at least the variable domain of a heavy chain (for camelid species) or at least the variable domains of a heavy chain and a light chain. Basic immunoglobulin structures in vertebrate systems are relatively well understood. See, e.g., Harlow et al., Antibodies: A Laboratory Manual, (Cold Spring Harbor Laboratory Press, 2nd ed. 1988). Unless otherwise stated, the term “antibody” encompasses anything ranging from a small antigen-binding fragment of an antibody to a full sized antibody, e.g., an IgG antibody that includes two complete heavy chains and two complete light chains, an IgA antibody that includes four complete heavy chains and four complete light chains and optionally includes a J-chain and/or a secretory component, or an IgM antibody that includes ten or twelve complete heavy chains and ten or twelve complete light chains and optionally includes a J-chain or functional fragment thereof.

The term “immunoglobulin” comprises various broad classes of polypeptides that can be distinguished biochemically. Those skilled in the art will appreciate that heavy chains are classified as gamma, mu, alpha, delta, or epsilon, (γ, μ, α, δ, ε) with some subclasses among them (e.g., γ1-γ4 or α1-α2). It is the nature of this chain that determines the “isotype” of the antibody as IgG, IgM, IgA, IgD, or IgE, respectively. The immunoglobulin subclasses (subtypes) e.g., IgG₁, IgG₂, IgG₃, IgG₄, IgA₁, IgA₂, etc. are well characterized and are known to confer functional specialization. Modified versions of each of these immunoglobulins are readily discernible to the skilled artisan in view of the instant disclosure and, accordingly, are within the scope of this disclosure.

Light chains are classified as either kappa or lambda (κ, λ). Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are expressed, e.g., by hybridomas, B cells or genetically engineered host cells. In the heavy chain, the amino acid sequences run from an N-terminus at the forked ends of the Y configuration to the C-terminus at the bottom of each chain. The basic structure of certain antibodies, e.g., IgG antibodies, includes two heavy chain subunits and two light chain subunits covalently connected via disulfide bonds to form a “Y” structure, also referred to herein as an “H2L2” structure, or a “binding unit.”

The term “binding unit” is used herein to refer to the portion of a binding molecule, e.g., an antibody, antibody-like molecule, antigen-binding fragment thereof, or multimerizing fragment thereof, which corresponds to a standard “H2L2” immunoglobulin structure, e.g., two heavy chains or fragments thereof and two light chains or fragments thereof. In certain aspects a binding unit can correspond to two heavy chains, e.g., in a camelid antibody. In certain aspects, e.g., where the binding molecule is a bivalent IgG antibody or antigen-binding fragment thereof, the terms “binding molecule” and “binding unit” are equivalent. In other aspects, e.g., where the binding molecule is multimeric, e.g., a dimeric IgA antibody or IgA-like antibody, a pentameric IgM antibody or IgM-like antibody, or a hexameric IgM antibody or IgM-like antibody, the binding molecule comprises two or more “binding units.” Two in the case of an IgA dimer, or five or six in the case of an IgM pentamer or hexamer, respectively. A binding unit need not include full-length antibody heavy and light chains, but will typically be bivalent, i.e., will include two “antigen-binding domains,” as defined above. As used herein, certain binding molecules provided in this disclosure are “dimeric,” and include two bivalent binding units that include IgA constant regions or multimerizing fragments thereof. Certain binding molecules provided in this disclosure are “pentameric” or “hexameric,” and include five or six bivalent binding units that include IgM constant regions or multimerizing fragments thereof. A binding molecule, e.g., an antibody or antibody-like molecule, comprising two or more, e.g., two, five, or six binding units, is referred to herein as “multimeric.”

The term “J-chain” as used herein refers to the J-chain of native sequence IgM or IgA antibodies of any animal species, any functional fragment thereof, derivative thereof, and/or variant thereof, including the mature human J-chain, the amino acid sequence of which is presented as SEQ ID NO: 2. Various J-chain variants and modified J-chain derivatives are disclosed herein. As persons of ordinary skill in the art will recognize, “a functional fragment” or a “functional variant” includes those fragments and variants that can associate with IgM heavy chain constant regions to form a pentameric IgM antibody (or alternatively can associate with IgA heavy chain constant regions to form a dimeric IgA antibody).

The term “modified J-chain” is used herein to refer to a derivative of a native sequence J-chain polypeptide comprising a heterologous moiety, e.g., a heterologous polypeptide, e.g., an extraneous binding domain introduced into the native sequence. The introduction can be achieved by any means, including direct or indirect fusion of the heterologous polypeptide or other moiety or by attachment through a peptide or chemical linker. The term “modified human J-chain” encompasses, without limitation, a native sequence human J-chain comprising the amino acid sequence of SEQ ID NO: 2 or functional fragment thereof, or functional variant thereof, modified by the introduction of a heterologous moiety, e.g., a heterologous polypeptide, e.g., an extraneous binding domain. In certain aspects the heterologous moiety does not interfere with efficient polymerization of IgM into a pentamer and binding of such polymers to a target. Exemplary modified J-chains can be found, e.g., in U.S. Pat. No. 9,951,134, in U.S. Patent Application Publication No. US-2019-0185570, and in U.S. Pat. No. 10,618,978, each of which is incorporated herein by reference in its entirety.

As used herein, the terms “IgM-derived binding molecule,” “IgM-like antibody,” “IgM-like binding unit,” or “IgM-like heavy chain constant region” refer to a variant antibody-derived binding molecule, antibody, binding unit, or heavy chain constant region that still retains the structural portions of an IgM heavy chain necessary to confer the ability to form multimers, e.g., hexamers, or in association with J-chain, form pentamers. An IgM-like antibody or IgM-derived binding molecule typically includes at least the Cμ4 and tailpiece (tp) domains of the IgM constant region but can include heavy chain constant region domains from other antibody isotypes, e.g., IgG, from the same species or from a different species. An IgM-like antibody or IgM-derived binding molecule can likewise be an antibody fragment in which one or more constant regions are deleted, as long as the IgM-like antibody is capable of forming hexamers and/or pentamers. Thus, an IgM-like antibody or IgM-derived binding molecule can be, e.g., a hybrid IgM/IgG antibody or can be a “multimerizing fragment” of an IgM antibody.

As used herein, the terms “IgA-derived binding molecule,” “IgA-like antibody,” “IgA-like binding unit,” or “IgA-like heavy chain constant region” refer to a variant antibody-derived binding molecule, antibody, binding unit, or heavy chain constant region that still retains the structural portions of an IgA heavy chain necessary to confer the ability to form multimers, e.g., dimers, in association with J-chain. An IgA-like antibody or IgA-derived binding molecule typically includes at least the Cα3 and tailpiece (tp) domains of the IgA constant region but can include heavy chain constant region domains from other antibody isotypes, e.g., IgG, from the same species or from a different species. An IgA-like antibody or IgA-derived binding molecule can likewise be an antibody fragment in which one or more constant regions are deleted, as long as the IgA-like antibody is capable of forming dimers in association with a J-chain. Thus, an IgA-like antibody or IgA-derived binding molecule can be, e.g., a hybrid IgA/IgG antibody or can be a “multimerizing fragment” of an IgA antibody.

The terms “valency,” “bivalent,” “multivalent” and grammatical equivalents, refer to the number of antigen-binding domains in given binding molecule, e.g., antibody or antibody-like molecule, or in a given binding unit. As such, the terms “bivalent”, “tetravalent”, and “hexavalent” in reference to a given binding molecule, e.g., an IgM antibody, IgM-like antibody or multimerizing fragment thereof, denote the presence of two antigen-binding domains, four antigen-binding domains, and six antigen-binding domains, respectively. A typical IgM antibody or IgM-like antibody or IgM-derived binding molecule where each binding unit is bivalent, can have 10 or 12 valencies. A bivalent or multivalent binding molecule, e.g., antibody or antibody-like molecule, can be monospecific, i.e., all of the antigen-binding domains are the same, or can be bispecific or multispecific, e.g., where two or more antigen-binding domains are different, e.g., bind to different epitopes on the same antigen, or bind to entirely different antigens.

The term “epitope” includes any molecular determinant capable of specific binding to an antigen-binding domain of an antibody or antibody-like molecule. In certain aspects, an epitope can include chemically active surface groupings of molecules such as amino acids, sugar side chains, phosphoryl, or sulfonyl, and, in certain aspects, can have a three-dimensional structural characteristic, and or specific charge characteristics. An epitope is a region of a target that is bound by an antigen-binding domain of an antibody.

The term “target” is used in the broadest sense to include substances that can be bound by a binding molecule, e.g., antibody or antibody-like molecule. A target can be, e.g., a polypeptide, a nucleic acid, a carbohydrate, a lipid, or other molecule. Moreover, a “target” can, for example, be a cell, an organ, or an organism that comprises an epitope that can be bound by a binding molecule, e.g., antibody or antibody-like molecule.

Both the light and heavy chains are divided into regions of structural and functional homology. The terms “constant” and “variable” are used functionally. The variable regions of both the light (VL) and heavy (VH) chains determine antigen recognition and specificity. Conversely, the constant domains of the light chain (CL) and the heavy chain (e.g., CH1, CH2, CH3, or CH4) confer biological properties such as secretion, transplacental mobility, Fc receptor binding, complement binding, and the like. By convention the numbering of the constant region domains increases as they become more distal from the antigen-binding site or amino-terminus of the antibody. The N-terminal portion is a variable region and at the C-terminal portion is a constant region; the CH3 (or CH4 in the case of IgM) and CL domains actually comprise the carboxy-terminus of the heavy and light chain, respectively.

A “full length IgM antibody heavy chain” is a polypeptide that includes, in N-terminal to C-terminal direction, an antibody heavy chain variable domain (VH), an antibody heavy chain constant domain 1 (CM1 or Cμ1), an antibody heavy chain constant domain 2 (CM2 or Cμ2), an antibody heavy chain constant domain 3 (CM3 or Cμ3), and an antibody heavy chain constant domain 4 (CM4 or Cμ4) that can include a tailpiece.

As indicated above, variable region(s) allows a binding molecule, e.g., antibody or antibody-like molecule, to selectively recognize and specifically bind epitopes on antigens. That is, the VL domain and VH domain, or subset of the complementarity determining regions (CDRs), of a binding molecule, e.g., an antibody or antibody-like molecule, combine to form the antigen-binding domain. More specifically, an antigen-binding domain can be defined by three CDRs on each of the VH and VL chains. Certain antibodies form larger structures. For example, IgA can form a molecule that includes two H2L2 binding units and a J-chain covalently connected via disulfide bonds, which can be further associated with a secretory component, and IgM can form a pentameric or hexameric molecule that includes five or six H2L2 binding units and optionally a J-chain covalently connected via disulfide bonds.

The six “complementarity determining regions” or “CDRs” present in an antibody antigen-binding domain are short, non-contiguous sequences of amino acids that are specifically positioned to form the antigen-binding domain as the antibody assumes its three-dimensional configuration in an aqueous environment. The remainder of the amino acids in the antigen-binding domain, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The antigen-binding domain formed by the positioned CDRs defines a surface complementary to the epitope on the target antigen. This complementary surface promotes the non-covalent binding of the antibody to its cognate epitope. The amino acids that make up the CDRs and the framework regions, respectively, can be readily identified for any given heavy or light chain variable region by one of ordinary skill in the art, since they have been defined in various different ways (see, “Sequences of Proteins of Immunological Interest,” Kabat, E., et al., U.S. Department of Health and Human Services, (1983); and Chothia and Lesk, J. Mol. Biol., 196:901-917 (1987), which are incorporated herein by reference in their entireties).

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. These particular regions have been described, for example, by Kabat et al., U.S. Dept. of Health and Human Services, “Sequences of Proteins of Immunological Interest” (1983) and by Chothia et al., J. Mol. Biol. 196:901-917 (1987), which are incorporated herein by reference. The Kabat and Chothia definitions include overlapping or subsets of amino acids when compared against each other. Nevertheless, application of either definition (or other definitions known to those of ordinary skill in the art) to refer to a CDR of an antibody or variant thereof is intended to be within the scope of the term as defined and used herein, unless otherwise indicated. The appropriate amino acids which encompass the CDRs as defined by each of the above cited references are set forth below in Table 1 as a comparison. The exact amino acid numbers which encompass a particular CDR will vary depending on the sequence and size of the CDR. Those skilled in the art can routinely determine which amino acids comprise a particular CDR given the variable region amino acid sequence of the antibody.

TABLE 1
CDR Definitions*

| | Kabat | Chothia |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

*Numbering of all CDR definitions in Table 1 is according to the numbering conventions set forth by Kabat et al. (see below).

Antibody variable domains can also be analyzed, e.g., using the IMGT information system (imgt_dot_cines_dot_fr/) (IMGT®/V-Quest) to identify variable region segments, including CDRs. (See, e.g., Brochet et al., Nucl. Acids Res., 36:W503-508, 2008).

Kabat et al. also defined a numbering system for variable region and constant region sequences that is applicable to any antibody. One of ordinary skill in the art can unambiguously assign this system of “Kabat numbering” to any variable region sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless use of the Kabat numbering system is explicitly noted, however, consecutive numbering is used for all amino acid sequences in this disclosure.

The Kabat numbering system for the human IgM constant domain can be found in Kabat, et al. “Tabulation and Analysis of Amino acid and nucleic acid Sequences of Precursors, V-Regions, C-Regions, J-Chain, T-Cell Receptors for Antigen, T-Cell Surface Antigens, β-2 Microglobulins, Major Histocompatibility Antigens, Thy-1, Complement, C-Reactive Protein, Thymopoietin, Integrins, Post-gamma Globulin, α-2 Macroglobulins, and Other Related Proteins,” U.S. Dept. of Health and Human Services (1991). IgM constant regions can be numbered sequentially (i.e., amino acid #1 starting with the first amino acid of the constant region, or by using the Kabat numbering scheme. A comparison of the numbering of two alleles of the human IgM constant region sequentially (presented herein as SEQ ID NO: 51 (allele IGHM*03) and SEQ ID NO: 52 (allele IGHM*04)) and by the Kabat system is set out below. The underlined amino acid residues are not accounted for in the Kabat system (“X,” double underlined below, can be serine (S) (SEQ ID NO: 51) or 1 cine (G) (SEQ ID NO: 52)):

| Sequential (SEQ ID NO: 51 or SEQ ID NO: 52)/ | |
|---|---|
| KABATnumbering key for IgM heavy chain | |
| 1/127 | GSASAPTLFP LVSCENSPSD TSSVAVGCLA QDFLPDSITF SWKYKNNSDI |
| 51/176 | SSTRGFPSVL RGGKYAATSQ VLLPSKDVMQ GTDEHVVCKV QHPNGNKEKN |
| 101/226 | VPLPVIAELP PKVSVFVPPR DGFFGNPRKS KLICQATGFS PRQIQVSWLR |
| 151/274 | EGKQVGSGVT TDQVQAEAKE SGPTTYKVTS TLTIKESDWL XQSMFTCRVD |
| 201/324 | HRGLTFQQNA SSMCVPDQDT AIRVFAIPPS FASIFLTKST KLTCLVTDLT |
| 251/374 | TYDSVTISWT RQNGEAVKTH TNISESHPNA TFSAVGEASI CEDDWNSGER |
| 301/424 | FTCTVTHTDL PSPLKQTISR PKGVALHRPD VYLLPPAREQ LNLRESATIT |
| 351/474 | CLVTGFSPAD VFVQWMQRGQ PLSPEKYVTS APMPEPQAPG RYFAHSILTV |
| 401/524 | SEEEWNTGET YTCVVAHEAL PNRVTERTVD KSTGKPTLYN VSLVMSDTAG |
| 451/574 | TCY |

Binding molecules, e.g., antibodies, antibody-like molecules, antigen-binding fragments, variants, or derivatives thereof, and/or multimerizing fragments thereof include, but are not limited to, polyclonal, monoclonal, human, humanized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VL or VH domain, fragments produced by a Fab expression library. scFv molecules are known in the art and are described, e.g., in U.S. Pat. No. 5,892,019.

By “specifically binds,” it is generally meant that a binding molecule, e.g., an antibody or fragment, variant, or derivative thereof binds to an epitope via its antigen-binding domain, and that the binding entails some complementarity between the antigen-binding domain and the epitope. According to this definition, a binding molecule, e.g., antibody or antibody-like molecule, is said to “specifically bind” to an epitope when it binds to that epitope, via its antigen-binding domain more readily than it would bind to a random, unrelated epitope. The term “specificity” is used herein to qualify the relative affinity by which a certain binding molecule binds to a certain epitope. For example, binding molecule “A” can be deemed to have a higher specificity for a given epitope than binding molecule “B,” or binding molecule “A” can be said to bind to epitope “C” with a higher specificity than it has for related epitope “D.”

A binding molecule, e.g., an antibody or fragment, variant, or derivative thereof disclosed herein can be said to bind a target antigen with an off rate (k(off)) of less than or equal to 5×10⁻² sec⁻¹, 10⁻² sec⁻¹, 5×10⁻³ sec⁻¹, 10⁻³ sec⁻¹, 5×10⁻⁴ sec⁻¹, 10⁻⁴ sec⁻¹, 5×10⁻⁵ sec⁻¹, or 10⁻⁵ sec⁻¹, 5×10⁻⁶ sec⁻¹, 10⁻⁶ sec⁻¹, 5×10⁻⁷ sec⁻¹ or 10⁻⁷ sec⁻¹.

A binding molecule, e.g., an antibody or antigen-binding fragment, variant, or derivative disclosed herein can be said to bind a target antigen with an on rate (k(on)) of greater than or equal to 10³ M⁻¹ sec⁻¹, 5×10³ M⁻¹ sec⁻¹, 10⁴ M⁻¹ sec⁻¹, 5×10⁴ M⁻¹ sec⁻¹, 10⁵ M⁻¹ sec⁻¹, 5×10⁵ M⁻¹ sec⁻¹, 10⁶ M⁻¹ sec⁻¹, or 5×10⁶ M⁻¹ sec⁻¹ or 10⁷ M⁻¹ sec⁻¹.

A binding molecule, e.g., an antibody or fragment, variant, or derivative thereof is said to competitively inhibit binding of a reference antibody or antigen-binding fragment to a given epitope if it preferentially binds to that epitope to the extent that it blocks, to some degree, binding of the reference antibody or antigen-binding fragment to the epitope. Competitive inhibition can be determined by any method known in the art, for example, competition ELISA assays. A binding molecule can be said to competitively inhibit binding of the reference antibody or antigen-binding fragment to a given epitope by at least 90%, at least 80%, at least 70%, at least 60%, or at least 50%.

As used herein, the term “affinity” refers to a measure of the strength of the binding of an individual epitope with one or more antigen-binding domains, e.g., of an immunoglobulin molecule. See, e.g., Harlow et al., Antibodies: A Laboratory Manual, (Cold Spring Harbor Laboratory Press, 2nd ed. 1988) at pages 27-28. As used herein, the term “avidity” refers to the overall stability of the complex between a population of antigen-binding domains and an antigen. See, e.g., Harlow at pages 29-34. Avidity is related to both the affinity of individual antigen-binding domains in the population with specific epitopes, and the valencies of the immunoglobulins and the antigen. For example, the interaction between a bivalent monoclonal antibody and an antigen with a highly repeating epitope structure, such as a polymer, would be one of high avidity. An interaction between a bivalent monoclonal antibody with a receptor present at a high density on a cell surface would also be of high avidity.

Binding molecules, e.g., antibodies or fragments, variants, or derivatives thereof as disclosed herein can also be described or specified in terms of their cross-reactivity. As used herein, the term “cross-reactivity” refers to the ability of a binding molecule, e.g., an antibody or fragment, variant, or derivative thereof, specific for one antigen, to react with a second antigen; a measure of relatedness between two different antigenic substances. Thus, a binding molecule is cross reactive if it binds to an epitope other than the one that induced its formation. The cross-reactive epitope generally contains many of the same complementary structural features as the inducing epitope, and in some cases, can actually fit better than the original.

A binding molecule, e.g., an antibody or fragment, variant, or derivative thereof can also be described or specified in terms of their binding affinity to an antigen. For example, a binding molecule can bind to an antigen with a dissociation constant or K_{D} no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹²M, 10⁻¹² M, 5×10⁻¹³ M, 10⁻¹³ M, 5×10⁻¹⁴M, 10⁻¹⁴ M, 5×10⁻¹⁵ M, or 10⁻¹⁵ M.

“Antigen-binding antibody fragments” including single-chain antibodies or other antigen-binding domains can exist alone or in combination with one or more of the following: hinge region, CH1, CH2, CH3, or CH4 domains, J-chain, or secretory component. Also included are antigen-binding fragments that can include any combination of variable region(s) with one or more of a hinge region, CH1, CH2, CH3, or CH4 domains, a J-chain, or a secretory component. Binding molecules, e.g., antibodies, or antigen-binding fragments thereof can be from any animal origin including birds and mammals. The antibodies can be, e.g., human, murine, donkey, rabbit, goat, guinea pig, camel, llama, horse, or chicken antibodies. In another embodiment, the variable region can be condricthoid in origin (e.g., from sharks). As used herein, “human” antibodies include antibodies having the amino acid sequence of a human immunoglobulin and include antibodies isolated from human immunoglobulin libraries or from animals transgenic for one or more human immunoglobulins and can in some instances express endogenous immunoglobulins and some not, as described infra and, for example in, U.S. Pat. No. 5,939,598 by Kucherlapati et al. According to aspects of the present disclosure, an IgM or IgM-like antibody or IgM-derived binding molecule as provided herein can include an antigen-binding fragment of an antibody, e.g., a scFv fragment, so long as the IgM or IgM-like antibody is able to form a multimer, e.g., a hexamer or a pentamer.

As used herein, the term “heavy chain subunit” includes amino acid sequences derived from an immunoglobulin heavy chain, a binding molecule, e.g., an antibody or antibody-like molecule comprising a heavy chain subunit can include at least one of: a VH domain, a CH1 domain, a hinge (e.g., upper, middle, and/or lower hinge region) domain, a CH2 domain, a CH3 domain, a CH4 domain, a tail-piece (tp), or a variant or fragment thereof. For example, a binding molecule, e.g., an antibody, antibody-like molecule, or fragment, variant, or derivative thereof can include without limitation, in addition to a VH domain: any combination of a CH1 domain, a hinge, a CH2 domain, a CH3 domain, a CH4 domain or a tailpiece (tp) of one or more antibody isotypes and/or species. In certain aspects a binding molecule, e.g., an antibody, antibody-like molecule, or fragment, variant, or derivative thereof can include, in addition to a VH domain, a CH3 domain and a CH4-tp domain; or a CH3 domain, a CH4-tp domain, and a J-chain. Further, a binding molecule, e.g., antibody or antibody-like molecule, for use in the disclosure can lack certain constant region portions, e.g., all or part of a CH2 domain. These domains (e.g., the heavy chain subunit) can be modified such that they vary in amino acid sequence from the original immunoglobulin molecule. According to aspects of the present disclosure, an IgM or IgM-like antibody as provided herein includes sufficient portions of an IgM heavy chain constant region to allow the IgM or IgM-like antibody to form a multimer, e.g., a hexamer or a pentamer, e.g., the IgM heavy chain constant region includes a “multimerizing fragment” of an IgM heavy chain constant region.

As used herein, the term “light chain subunit” includes amino acid sequences derived from an immunoglobulin light chain. The light chain subunit includes at least a VL, and can further include a CL (e.g., Cκ or Cλ) domain.

Binding molecules, e.g., antibodies, antibody-like molecules, antigen-binding fragments, variants, or derivatives thereof, or multimerizing fragments thereof can be described or specified in terms of the epitope(s) or portion(s) of an antigen that they recognize or specifically bind. The portion of a target antigen that specifically interacts with the antigen-binding domain of an antibody is an “epitope,” or an “antigenic determinant.” A target antigen can comprise a single epitope or at least two epitopes, and can include any number of epitopes, depending on the size, conformation, and type of antigen.

As used herein, the term “hinge region” includes the portion of a heavy chain molecule that joins the CH1 domain to the CH2 domain in IgG, IgA, and IgD heavy chains. This hinge region comprises approximately 25 amino acids and is flexible, thus allowing the two N-terminal antigen-binding regions to move independently.

As used herein the term “disulfide bond” includes the covalent bond formed between two sulfur atoms. The amino acid cysteine comprises a thiol group that can form a disulfide bond or bridge with a second thiol group.

As used herein, the term “chimeric antibody” refers to an antibody in which the immunoreactive region or site is obtained or derived from a first species and the constant region (which can be intact, partial or modified) is obtained from a second species. In some embodiments the target binding region or site will be from a non-human source (e.g. mouse or primate) and the constant region is human.

The terms “multispecific antibody” or “bispecific antibody” refer to an antibody or antibody-like molecule that has antigen-binding domains for two or more different epitopes within a single antibody molecule. Other binding molecules in addition to the canonical antibody structure can be constructed with two binding specificities.

As used herein, the term “engineered antibody” refers to an antibody in which the variable domain in either the heavy and light chain or both is altered by at least partial replacement of one or more amino acids in either the CDR or framework regions. In certain aspects entire CDRs from an antibody of known specificity can be grafted into the framework regions of a heterologous antibody. Although alternate CDRs can be derived from an antibody of the same class or even subclass as the antibody from which the framework regions are derived, CDRs can also be derived from an antibody of different class, e.g., from an antibody from a different species. An engineered antibody in which one or more “donor” CDRs from a non-human antibody of known specificity are grafted into a human heavy or light chain framework region is referred to herein as a “humanized antibody.” In certain aspects not all of the CDRs are replaced with the complete CDRs from the donor variable region and yet the antigen-binding capacity of the donor can still be transferred to the recipient variable domains. Given the explanations set forth in, e.g., U.S. Pat. Nos. 5,585,089, 5,693,761, 5,693,762, and 6,180,370, it will be well within the competence of those skilled in the art, either by carrying out routine experimentation to obtain a functional engineered or humanized antibody.

As used herein the term “engineered” includes manipulation of nucleic acid or polypeptide molecules by synthetic means (e.g. by recombinant techniques, in vitro peptide synthesis, by enzymatic or chemical coupling of peptides, nucleic acids, or glycans, or some combination of these techniques).

As used herein, the terms “linked,” “fused” or “fusion” or other grammatical equivalents can be used interchangeably. These terms refer to the joining together of two more elements or components, by whatever means including chemical conjugation or recombinant means. An “in-frame fusion” refers to the joining of two or more polynucleotide open reading frames (ORFs) to form a continuous longer ORF, in a manner that maintains the translational reading frame of the original ORFs. Thus, a recombinant fusion protein is a single protein containing two or more segments that correspond to polypeptides encoded by the original ORFs (which segments are not normally so joined in nature.) Although the reading frame is thus made continuous throughout the fused segments, the segments can be physically or spatially separated by, for example, in-frame linker sequence. For example, polynucleotides encoding the CDRs of an immunoglobulin variable region can be fused, in-frame, but be separated by a polynucleotide encoding at least one immunoglobulin framework region or additional CDR regions, as long as the “fused” CDRs are co-translated as part of a continuous polypeptide.

In the context of polypeptides, a “linear sequence” or a “sequence” is an order of amino acids in a polypeptide in an amino to carboxyl terminal direction in which amino acids that neighbor each other in the sequence are contiguous in the primary structure of the polypeptide. A portion of a polypeptide that is “amino-terminal” or “N-terminal” to another portion of a polypeptide is that portion that comes earlier in the sequential polypeptide chain. Similarly, a portion of a polypeptide that is “carboxy-terminal” or “C-terminal” to another portion of a polypeptide is that portion that comes later in the sequential polypeptide chain. For example, in a typical antibody, the variable domain is “N-terminal” to the constant region, and the constant region is “C-terminal” to the variable domain.

The term “expression” as used herein refers to a process by which a gene produces a biochemical, for example, a polypeptide. The process includes any manifestation of the functional presence of the gene within the cell including, without limitation, gene knockdown as well as both transient expression and stable expression. It includes without limitation transcription of the gene into RNA, e.g., messenger RNA (mRNA), and the translation of such mRNA into polypeptide(s). If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide that is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

Terms such as “treating” or “treatment” or “to treat” or “alleviating” or “to alleviate” refer to therapeutic measures that cure, slow down, lessen symptoms of, and/or halt or slow the progression of an existing diagnosed disease, pathologic condition, or disorder. Terms such as “prevent,” “prevention,” “avoid,” “deterrence” and the like refer to prophylactic or preventative measures that prevent the development of an undiagnosed targeted disease, pathologic condition, or disorder. Thus, “a subject in need of treatment” can include subjects already with the disorder; those prone to have the disorder; and those in whom the disorder is to be prevented.

As used herein the terms “serum half-life” or “plasma half-life” refer to the time it takes (e.g., in minutes, hours, or days) following administration for the serum or plasma concentration of a protein or a drug, e.g., a binding molecule such as an antibody, antibody-like molecule or fragment thereof as described herein, to be reduced by 50%. Two half-lives can be described: the alpha half-life, a half-life, or ti/2a, which is the rate of decline in plasma concentrations due to the process of drug redistribution from the central compartment, e.g., the blood in the case of intravenous delivery, to a peripheral compartment (e.g., a tissue or organ), and the beta half-life, R half-life, or ti/20 which is the rate of decline due to the processes of excretion or metabolism.

As used herein the term “area under the plasma drug concentration-time curve” or “AUC” reflects the actual body exposure to drug after administration of a dose of the drug and is expressed in mg*h/L. This area under the curve is measured from time 0 (t₀) to infinity (∞) and is dependent on the rate of elimination of the drug from the body and the dose administered.

As used herein, the term “mean residence time” or “MRT” refers to the average length of time the drug remains in the body.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, swine, cows, bears, and so on.

As used herein, phrases such as “a subject that would benefit from therapy” and “an animal in need of treatment” refers to a subset of subjects, from amongst all prospective subjects, which would benefit from administration of a given therapeutic agent, e.g., a binding molecule such as an antibody or antibody-like molecule, comprising one or more antigen-binding domains. Such binding molecules, e.g., antibodies or antibody-like molecules, can be used, e.g., for a diagnostic procedure and/or for treatment or prevention of a disease.

IgM Antibodies, IgM-Like Antibodies, and IgM-Derived Binding Molecules

IgM is the first immunoglobulin produced by B cells in response to stimulation by antigen and is naturally present at around 1.5 mg/ml in serum with a half-life of about 5 days. IgM is a pentameric or hexameric molecule and thus includes five or six binding units. An IgM binding unit typically includes two light and two heavy chains. While an IgG heavy chain constant region contains three heavy chain constant domains (CH1, CH2 and CH3), the heavy (μ) constant region of IgM additionally contains a fourth constant domain (CH4) and includes a C-terminal “tailpiece” (tp). While several human alleles exist, the human IgM constant region typically comprises the amino acid sequence SEQ ID NO: 51 (IMGT allele IGHM*03, identical to, e.g., GenBank Accession No. pir∥S37768) or SEQ ID NO: 52 (IMGT allele IGHM*04, identical to, e.g., GenBank Accession No. sp|P01871.4). The human Cμ1 region ranges from about amino acid 5 to about amino acid 102 of SEQ ID NO: 51 or SEQ ID NO: 52; the human Cμ2 region ranges from about amino acid 114 to about amino acid 205 of SEQ ID NO: 51 or SEQ ID NO: 52, the human Cμ3 region ranges from about amino acid 224 to about amino acid 319 of SEQ ID NO: 51 or SEQ ID NO: 52, the Cμ4 region ranges from about amino acid 329 to about amino acid 430 of SEQ ID NO: 51 or SEQ ID NO: 52, and the tailpiece ranges from about amino acid 431 to about amino acid 453 of SEQ ID NO: 51 or SEQ ID NO: 52.

Other forms of the human IgM constant region with minor sequence variations exist, including, without limitation, GenBank Accession Nos. CAB37838.1 and pir∥MHHU. The amino acid substitutions, insertions, and/or deletions at positions corresponding to SEQ ID NO: 51 or SEQ ID NO: 52 described and claimed elsewhere in this disclosure can likewise be incorporated into alternate human IgM sequences, as well as into IgM constant region amino acid sequences of other species.

Each IgM heavy chain constant region can be associated with an antigen-binding domain, e.g., a scFv or VHH, or a subunit of an antigen-binding domain, e.g., a VH region.

Five IgM binding units can form a complex with an additional small polypeptide chain (the J-chain), or a functional fragment, variant, or derivative thereof, to form a pentameric IgM antibody or IgM-like antibody. The precursor form of the human J-chain is presented as SEQ ID NO: 1. The signal peptide (underlined in Table 10) extends from amino acid 1 to about amino acid 22 of SEQ ID NO: 1, and the mature human J-chain extends from about amino acid 23 to amino acid 159 of SEQ ID NO: 1. The mature human J-chain has the amino acid sequence SEQ ID NO: 2.

Exemplary variant and modified J-chains are provided elsewhere herein. Without the J-chain, an IgM antibody or IgM-like antibody typically assembles into a hexamer, comprising six binding units and up to twelve antigen-binding domains. With a J-chain, an IgM antibody or IgM-like antibody typically assembles into a pentamer, comprising five binding units and up to ten antigen-binding domains, or more, if the J-chain is a modified J-chain comprising one or more heterologous polypeptides that can be, e.g., additional antigen-binding domain(s). The assembly of five or six IgM binding units into a pentameric or hexameric IgM antibody or IgM-like antibody is thought to involve interactions between the Cμ4 and tailpiece domains. See, e.g., Braathen, R., et al., J Biol. Chem. 277:42755-42762 (2002). Accordingly, the constant regions of a pentameric or hexameric IgM antibody or antibody-like molecule provided in this disclosure typically includes at least the Cμ4 and/or tailpiece domains (also referred to herein collectively as Cμ4-tp). A “multimerizing fragment” of an IgM heavy chain constant region thus includes at least the Cμ4-tp domain. An IgM heavy chain constant region can additionally include a Cμ3 domain or a fragment thereof, a Cμ2 domain or a fragment thereof, a Cμ1 domain or a fragment thereof. In certain aspects, a binding molecule, e.g., an IgM antibody or IgM-like antibody as provided herein can include a complete IgM heavy (μ) chain constant domain, e.g., SEQ ID NO: 51 or SEQ ID NO: 52, or a variant, derivative, or analog thereof, e.g., as provided herein.

In certain aspects, the disclosure provides a pentameric IgM or IgM-like antibody comprising five bivalent binding units, where each binding unit includes two IgM heavy chain constant regions or multimerizing fragments or variants thereof, each associated with an antigen-binding domain or a subunit of an antigen-binding domain. In certain aspects, the two IgM heavy chain constant regions are human heavy chain constant regions.

Where the IgM or IgM-like antibody provided herein is pentameric, the IgM or IgM-like antibody typically further includes a J-chain, or functional fragment or variant thereof. In certain aspects the J-chain is a modified J-chain or variant thereof that further comprises one or more heterologous moieties attached thereto, as described elsewhere herein. In certain aspects the J-chain can be mutated to affect, e.g., enhance, the serum half-life of the IgM or IgM-like antibody provided herein, as discussed elsewhere herein. In certain embodiments the J-chain can be mutated to affect glycosylation, as discussed elsewhere in this disclosure.

In some embodiments, the multimeric binding molecules are hexameric and comprise six bivalent binding units or variants or fragments thereof. In some embodiments, the multimeric binding molecules are hexameric and comprise six bivalent binding units or variants or fragments thereof, where each binding unit comprises two IgM heavy chain constant regions or multimerizing fragments or variants thereof.

An IgM heavy chain constant region can include one or more of a Cμ1 domain or fragment or variant thereof, a Cμ2 domain or fragment or variant thereof, a Cμ3 domain or fragment or variant thereof, a Cμ4 domain or fragment or variant thereof, and/or a tail piece (tp) or fragment or variant thereof, provided that the constant region can serve a desired function in the IgM or IgM-like antibody, e.g., associate with second IgM constant region to form a binding unit with one, two, or more antigen-binding domain(s), and/or associate with other binding units (and in the case of a pentamer, a J-chain) to form a hexamer or a pentamer. In certain embodiments the two IgM heavy chain constant regions or fragments or variants thereof within an individual binding unit each comprise a Cμ4 domain or fragment or variant thereof, a tailpiece (tp) or fragment or variant thereof, or a combination of a Cμ4 domain and a tp or fragment or variant thereof. In certain embodiments the two IgM heavy chain constant regions or fragments or variants thereof within an individual binding unit each further comprise a Cμ3 domain or fragment or variant thereof, a Cμ2 domain or fragment or variant thereof, a Cμ1 domain or fragment or variant thereof, or any combination thereof.

In some embodiments, the binding units of the IgM or IgM-like antibody comprise two light chains. In some embodiments, the binding units of the IgM or IgM-like antibody comprise two fragments of light chains. In some embodiments, the light chains are kappa light chains. In some embodiments, the light chains are lambda light chains. In some embodiments, each binding unit comprises two immunoglobulin light chains each comprising a VL situated amino terminal to an immunoglobulin light chain constant region.

IgM Antibodies, IgM-Like Antibodies, and IgM-Derived Binding Molecules with Enhanced Serum Half-Life

Certain IgM-derived multimeric binding molecules provided herein can be modified to have enhanced serum half-life. Exemplary IgM heavy chain constant region mutations that can enhance serum half-life of an IgM-derived binding molecule are disclosed in PCT Publication No. WO 2019/169314A1, which is incorporated by reference herein in its entirety. For example, a variant IgM heavy chain constant region of an IgM-derived binding molecule as provided herein can include an amino acid substitution at an amino acid position corresponding to amino acid S401, E402, E403, R344, and/or E345 of a wild-type human IgM constant region (e.g., SEQ ID NO: 51 or SEQ ID NO: 52). By “an amino acid corresponding to amino acid S401, E402, E403, R344, and/or E345 of a wild-type human IgM constant region” is meant the amino acid in the sequence of the IgM constant region of any species which is homologous to S401, E402, E403, R344, and/or E345 in the human IgM constant region. In certain aspects, the amino acid corresponding to S401, E402, E403, R344, and/or E345 of SEQ ID NO: 51 or SEQ ID NO: 52 can be substituted with any amino acid, e.g., alanine.

IgM Antibodies, IgM-Like Antibodies, and IgM-Derived Binding Molecules with Reduced CDC Activity

Certain IgM-derived multimeric binding molecules as provided herein can be engineered to exhibit reduced complement-dependent cytotoxicity (CDC) activity to cells in the presence of complement, relative to a reference IgM antibody or IgM-like antibody with a corresponding reference human IgM constant region identical, except for the mutations conferring reduced CDC activity. These CDC mutations can be combined with any of the mutations to block N-linked glycosylation and/or to confer increased serum half-life as provided herein. By “corresponding reference human IgM constant region” is meant a human IgM constant region or portion thereof, e.g., a Cμ3 domain, that is identical to the variant IgM constant region except for the modification or modifications in the constant region affecting CDC activity. In certain aspects, the variant human IgM constant region includes one or more amino acid substitutions, e.g., in the Cμ3 domain, relative to a wild-type human IgM constant region as described, e.g., in PCT Publication No. WO/2018/187702, which is incorporated herein by reference in its entirety. Assays for measuring CDC are well known to those of ordinary skill in the art, and exemplary assays are described e.g., in PCT Publication No. WO/2018/187702.

In certain embodiments, a variant human IgM constant region conferring reduced CDC activity includes an amino acid substitution corresponding to the wild-type human IgM constant region at position L310, P311, P313, and/or K315 of SEQ ID NO: 22 (human IgM constant region allele IGHM*03) or SEQ ID NO: 23 (human IgM constant region allele IGHM*04). In certain aspects, a variant human IgM constant region conferring reduced CDC activity includes an amino acid substitution corresponding to the wild-type human IgM constant region at position P311 of SEQ ID NO: 51 or SEQ ID NO: 52. In other aspects the variant IgM constant region as provided herein contains an amino acid substitution corresponding to the wild-type human IgM constant region at position P313 of SEQ ID NO: 51 or SEQ ID NO: 52. In other aspects the variant IgM constant region as provided herein contains a combination of substitutions corresponding to the wild-type human IgM constant region at positions P311 of SEQ ID NO: 51 or SEQ ID NO: 52 and/or P313 of SEQ ID NO: 51 or SEQ ID NO: 52. These proline residues can be independently substituted with any amino acid, e.g., with alanine, serine, or glycine. In certain embodiments, a variant human IgM constant region conferring reduced CDC activity includes an amino acid substitution corresponding to the wild-type human IgM constant region at position K315 of SEQ ID NO: 22 or SEQ ID NO: 23. The lysine residue can be independently substituted with any amino acid, e.g., with alanine, serine, glycine, or aspartic acid. In certain embodiments, a variant human IgM constant region conferring reduced CDC activity includes an amino acid substitution corresponding to the wild-type human IgM constant region at position K315 of SEQ ID NO: 22 or SEQ ID NO: 23 with aspartic acid. In certain embodiments, a variant human IgM constant region conferring reduced CDC activity includes an amino acid substitution corresponding to the wild-type human IgM constant region at position L310 of SEQ ID NO: 22 or SEQ ID NO: 23. The lysine residue can be independently substituted with any amino acid, e.g., with alanine, serine, glycine, or aspartic acid. In certain embodiments, a variant human IgM constant region conferring reduced CDC activity includes an amino acid substitution corresponding to the wild-type human IgM constant region at position L310 of SEQ ID NO: 22 or SEQ ID NO: 23 with aspartic acid.

Human and certain non-human primate IgM constant regions typically include five (5) naturally-occurring asparagine (N)-linked glycosylation motifs or sites. As used herein “an N-linked glycosylation motif” comprises or consists of the amino acid sequence N-X₁-S/T, where N is asparagine, X₁ is any amino acid except proline (P), and S/T is serine (S) or threonine (T). The glycan is attached to the nitrogen atom of the asparagine residue. See, e.g., Drickamer K, Taylor M E (2006), Introduction to Glycobiology (2nd ed.). Oxford University Press, USA. N-linked glycosylation motifs occur in the human IgM heavy chain constant regions of SEQ ID NO: 22 or SEQ ID NO: 23 starting at positions 46 (“N1”), 209 (“N2”), 272 (“N3”), 279 (“N4”), and 440 (“N5”). These five motifs are conserved in non-human primate IgM heavy chain constant regions, and four of the five are conserved in the mouse IgM heavy chain constant region. Accordingly, in some embodiments, IgM heavy chain constant regions of a multimeric binding molecule as provided herein comprise 5 N-linked glycosylation motifs: N1, N2, N3, N4, and N5. In some embodiments, at least three of the N-linked glycosylation motifs (e.g., N1, N2, and N3) on each IgM heavy chain constant region are occupied by a complex glycan.

In certain embodiments, at least one, at least two, at least three, or at least four of the N-X₁-S/T motifs can include an amino acid insertion, deletion, or substitution that prevents glycosylation at that motif. In certain embodiments, the IgM-derived multimeric binding molecule can include an amino acid insertion, deletion, or substitution at motif N1, motif N2, motif N3, motif N5, or any combination of two or more, three or more, or all four of motifs N1, N2, N3, or N5, where the amino acid insertion, deletion, or substitution prevents glycosylation at that motif. In some embodiment, the IgM constant region comprises one or more substitutions relative to a wild-type human IgM constant region at positions 46, 209, 272, or 440 of SEQ ID NO: 22 (human IgM constant region allele IGHM*03) or SEQ ID NO: 23 (human IgM constant region allele IGHM*04). See, e.g., U.S. Provisional Application No. 62/891,263, which is incorporated herein by reference in its entirety.

IgA Antibodies, IgA-Like Antibodies, and IgA-Derived Binding Molecules

IgA plays a critical role in mucosal immunity and comprises about 15% of total immunoglobulin produced. IgA can be monomeric or multimeric, forming primarily dimeric molecules, but can also assemble as trimers, tetramers, and/or pentamers. See, e.g., de Sousa-Pereira, P., and J. M. Woof, Antibodies 8:57 (2019).

In some embodiments, the multimeric binding molecules are dimeric and comprise two bivalent binding units or variants or fragments thereof. In some embodiments, the multimeric binding molecules are dimeric, comprise two bivalent binding units or variants or fragments thereof, and further comprise a J-chain or functional fragment or variant thereof as described herein. In some embodiments, the multimeric binding molecules are dimeric, comprise two bivalent binding units or variants or fragments thereof, and further comprise a J-chain or functional fragment or variant thereof as described herein, where each binding unit comprises two IgA heavy chain constant regions or multimerizing fragments or variants thereof.

In some embodiments, the multimeric binding molecules are tetrameric and comprise four bivalent binding units or variants or fragments thereof. In some embodiments, the multimeric binding molecules are tetrameric, comprise four bivalent binding units or variants or fragments thereof, and further comprise a J-chain or functional fragment or variant thereof as described herein. In some embodiments, the multimeric binding molecules are tetrameric, comprise four bivalent binding units or variants or fragments thereof, and further comprise a J-chain or functional fragment or variant thereof as described herein, where each binding unit comprises two IgA heavy chain constant regions or multimerizing fragments or variants thereof.

In certain aspects, the multimeric binding molecule provided by this disclosure is a dimeric binding molecule that includes IgA heavy chain constant regions, or multimerizing fragments thereof, each associated with an antigen-binding domain for a total of four antigen-binding domains. As provided herein, an IgA antibody, IgA-derived binding molecule, or IgA-like antibody includes two binding units and a J-chain, e.g., a modified J-chain comprising IL-15 and/or the IL-15 receptor-α sushi domain fused thereto as described elsewhere herein. Each binding unit as provided comprises two IgA heavy chain constant regions or multimerizing fragments or variants thereof. In certain aspects, at least three or all four antigen-binding domains of the multimeric binding molecule bind to the same target antigen. In certain aspects, at least three or all four binding polypeptides of the multimeric binding molecule are identical.

A bivalent IgA-derived binding unit includes two IgA heavy chain constant regions, and a dimeric IgA-derived binding molecule includes two binding units. IgA contains the following heavy chain constant domains, Cal (or alternatively CA1 or CH1), a hinge region, Cα2 (or alternatively CA2 or CH2), and Cα3 (or alternatively CA3 or CH3), and a C-terminal “tailpiece.” Human IgA has two subtypes, IgA1 and IgA2. The human IgA1 constant region typically includes the amino acid sequence SEQ ID NO: 53 The human Cal domain extends from about amino acid 6 to about amino acid 98 of SEQ ID NO: 53; the human IgA1 hinge region extends from about amino acid 102 to about amino acid 124 of SEQ ID NO:53, the human Cα2 domain extends from about amino acid 125 to about amino acid 219 of SEQ ID NO:53, the human Cα3 domain extends from about amino acid 228 to about amino acid 330 of SEQ ID NO:53, and the tailpiece extends from about amino acid 331 to about amino acid 352 of SEQ ID NO:53. The human IgA2 constant region typically includes the amino acid sequence SEQ ID NO:54. The human Cal domain extends from about amino acid 6 to about amino acid 98 of SEQ ID NO:54; the human IgA2 hinge region extends from about amino acid 102 to about amino acid 111 of SEQ ID NO:54, the human Cα2 domain extends from about amino acid 113 to about amino acid 206 of SEQ ID NO:54, the human Cα3 domain extends from about amino acid 215 to about amino acid 317 of SEQ ID NO:54, and the tailpiece extends from about amino acid 318 to about amino acid 340 of SEQ ID NO:54.

Two IgA binding units can form a complex with two additional polypeptide chains, the J chain (SEQ ID NO: 2) and the secretory component (precursor, SEQ ID NO: 55, mature, SEQ ID NO: 56) to form a bivalent secretory IgA (sIgA)-derived binding molecule as provided herein. While not wishing to be bound by theory, the assembly of two IgA binding units into a dimeric IgA-derived binding molecule is thought to involve the Cα3 and tailpiece domains. See, e.g., Braathen, R., et al., J. Biol. Chem. 277:42755-42762 (2002). Accordingly, a multimerizing dimeric IgA-derived binding molecule provided in this disclosure typically includes IgA constant regions that include at least the Cα3 and tailpiece domains. Four IgA binding units can likewise form a tetramer complex with a J-chain. A sIgA antibody can also form as a higher order multimer, e.g., a tetramer.

An IgA heavy chain constant region can additionally include a Cα2 domain or a fragment thereof, an IgA hinge region or fragment thereof, a Cal domain or a fragment thereof, and/or other IgA (or other immunoglobulin, e.g., IgG) heavy chain domains, including, e.g., an IgG hinge region. In certain aspects, a binding molecule as provided herein can include a complete IgA heavy (α) chain constant domain (e.g., SEQ ID NO:53 or SEQ ID NO:54), or a variant, derivative, or analog thereof. In some embodiments, the IgA heavy chain constant regions or multimerizing fragments thereof are human IgA constant regions.

In certain aspects each binding unit of a multimeric binding molecule as provided herein includes two IgA heavy chain constant regions or multimerizing fragments or variants thereof, each including at least an IgA Cα3 domain and an IgA tailpiece domain. In certain aspects the IgA heavy chain constant regions can each further include an IgA Cα2 domain situated N-terminal to the IgA Cα3 and IgA tailpiece domains. For example, the IgA heavy chain constant regions can include amino acids 125 to 353 of SEQ ID NO:53 or amino acids 113 to 340 of SEQ ID NO:54. In certain aspects the IgA heavy chain constant regions can each further include an IgA or IgG hinge region situated N-terminal to the IgA Cα2 domains. For example, the IgA heavy chain constant regions can include amino acids 102 to 353 of SEQ ID NO:53 or amino acids 102 to 340 of SEQ ID NO:54. In certain aspects the IgA heavy chain constant regions can each further include an IgA Cal domain situated N-terminal to the IgA hinge region.

In some embodiments, each binding unit of an IgA antibody, IgA-like antibody, or other IgA-derived binding molecule comprises two light chains. In some embodiments, each binding unit of an IgA antibody, IgA-like antibody, or other IgA-derived binding molecule comprises two fragments light chains. In some embodiments, the light chains are kappa light chains. In some embodiments, the light chains are lambda light chains. In some embodiments the light chains are chimeric kappa-lambda light chains. In some embodiments, each binding unit comprises two immunoglobulin light chains each comprising a VL situated amino terminal to an immunoglobulin light chain constant region.

Modified and/or Variant J-Chains

In certain aspects, the J-chain of a pentameric an IgM or IgM-like antibody or a dimeric IgA or IgA-like antibody as provided herein can be modified, e.g., by introduction of a heterologous moiety, or two or more heterologous moieties, e.g., polypeptides, without interfering with the ability of the IgM or IgM-like antibody or IgA or IgA-like antibody to assemble and bind to its binding target(s). See U.S. Pat. No. 9,951,134, PCT Publication No. WO 2017/059387, and PCT Publication No. WO 2017/059380, each of which is incorporated herein by reference in its entirety. Accordingly, IgM or IgM-like antibodies or IgA or IgA-like antibodies as provided herein, including multispecific IgM or IgM-like antibodies as described elsewhere herein, can include a modified J-chain or functional fragment or variant thereof that further includes a heterologous moiety, e.g., a heterologous polypeptide, introduced into the J-chain or fragment or variant thereof. In certain aspects heterologous moiety can be a peptide or polypeptide fused in frame or chemically conjugated to the J-chain or fragment or variant thereof. For example, the heterologous polypeptide can be fused to the J-chain or functional fragment or variant thereof. In certain aspects, the heterologous polypeptide is fused to the J-chain or functional fragment or variant thereof via a linker, e.g., a peptide linker consisting of least 5 amino acids, but typically no more than 25 amino acids. In certain aspects, the peptide linker consists of GGGGS (SEQ ID NO: 80), GGGGSGGGGS (SEQ ID NO: 81), GGGGSGGGGSGGGGS (SEQ ID NO: 78), GGGGSGGGGSGGGGSGGGGS (SEQ ID NO: 82), or GGGGSGGGGSGGGGSGGGGSGGGGS (SEQ ID NO: 83), or GGSGGGGSGGGSGGGGSLQ (SEQ ID NO: 79). In certain aspects the heterologous moiety can be a chemical moiety conjugated to the J-chain. Heterologous moieties to be attached to a J-chain can include, without limitation, a binding moiety, e.g., an antibody or antigen-binding fragment thereof, e.g., a single chain Fv (scFv) molecule, a stabilizing peptide that can increase the half-life of the IgM or IgM-like antibody, or a chemical moiety such as a polymer or a cytotoxin. In some embodiments, heterologous moiety comprises a stabilizing peptide that can increase the half-life of the binding molecule, e.g., human serum albumin (HSA) or an HSA binding molecule.

In some embodiments, a modified J-chain can further include an antigen-binding domain, e.g., a polypeptide capable of specifically binding to a target antigen. In certain aspects, an antigen-binding domain associated with a modified J-chain can be an antibody or an antigen-binding fragment thereof, as described elsewhere herein. In certain aspects the antigen-binding domain can be a single chain Fv (scFv) antigen-binding domain or a single-chain antigen-binding domain derived, e.g., from a camelid or condricthoid antibody. The antigen-binding domain can be introduced into the J-chain at any location that allows the binding of the antigen-binding domain to its binding target without interfering with J-chain function or the function of an associated IgM or IgA antibody. Insertion locations include but are not limited to at or near the C-terminus, at or near the N-terminus or at an internal location that, based on the three-dimensional structure of the J-chain, is accessible. In certain aspects, the antigen-binding domain can be introduced into the mature human J-chain of SEQ ID NO: 2 between cysteine residues 92 and 101 of SEQ ID NO: 2. In a further aspect, the antigen-binding domain can be introduced into the human J-chain of SEQ ID NO: 2 at or near a glycosylation site. In a further aspect, the antigen-binding domain can be introduced into the human J-chain of SEQ ID NO: 2 within about 10 amino acid residues from the C-terminus, or within about 10 amino acids from the N-terminus.

In some embodiments described in detail herein, a modified J-chain can further include a cytokine, e.g., interleukin-2 (IL-2) or interleukin-15 (IL-15), or a receptor-binding fragment or variant thereof, which in certain aspects can be associated, either via binding or covalent attachment, to part of its receptor, e.g., the sushi domain of IL-15 receptor-α.

In certain aspects, the J-chain of an IgM antibody, IgM-like antibody or IgM-derived binding molecule as provided herein is a variant J-chain that comprises one or more amino acid substitutions that can alter, e.g., the serum half-life of an IgM antibody, IgM-like antibody, IgA antibody, IgA-like antibody, or IgM- or IgA-derived binding molecule provided herein. For example certain amino acid substitutions, deletions, or insertions can result in the IgM-derived binding molecule exhibiting an increased serum half-life upon administration to a subject animal relative to a reference IgM-derived binding molecule that is identical except for the one or more single amino acid substitutions, deletions, or insertions in the variant J-chain, and is administered using the same method to the same animal species. In certain embodiments the variant J-chain can include one, two, three, or four single amino acid substitutions, deletions, or insertions relative to the reference J-chain.

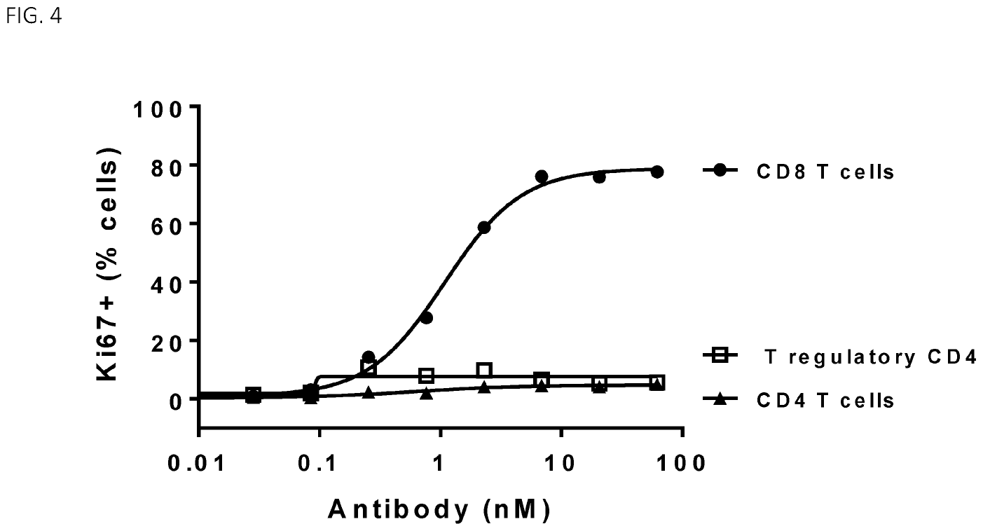

In some embodiments, the multimeric binding molecule can comprise a variant J-chain sequence, such as a variant sequence described herein with reduced glycosylation or reduced binding to one or more polymeric Ig receptors (e.g., pIgR, Fc alpha-mu receptor (FcαμR), or Fc mu receptor (FcμR)). See, e.g., PCT Publication No. WO 2019/169314, which is incorporated herein by reference in its entirety. In certain embodiments, the variant J-chain can comprise an amino acid substitution at the amino acid position corresponding to amino acid Y102 of the mature wild-type human J-chain (SEQ ID NO: 2). By “an amino acid corresponding to amino acid Y102 of the mature wild-type human J-chain” is meant the amino acid in the sequence of the J-chain of any species which is homologous to Y102 in the human J-chain. See PCT Publication No. WO 2019/169314A1, which is incorporated herein by reference in its entirety. The position corresponding to Y102 in SEQ ID NO: 2 is conserved in the J-chain amino acid sequences of at least 43 other species. See FIG. 4 of U.S. Pat. No. 9,951,134, which is incorporated by reference herein. Certain mutations at the position corresponding to Y102 of SEQ ID NO: 2 can inhibit the binding of certain immunoglobulin receptors, e.g., the human or murine Fcαμ receptor, the murine Fcμ receptor, and/or the human or murine polymeric Ig receptor (pIg receptor) to an IgM pentamer comprising the mutant J-chain. IgM antibodies, IgM-like antibodies, and IgM-derived binding molecules comprising a mutation at the amino acid corresponding to Y102 of SEQ ID NO: 2 have an improved serum half-life when administered to an animal than a corresponding antibody, antibody-like molecule or binding molecule that is identical except for the substitution, and which is administered to the same species in the same manner. In certain aspects, the amino acid corresponding to Y102 of SEQ ID NO: 2 can be substituted with any amino acid. In certain aspects, the amino acid corresponding to Y102 of SEQ ID NO: 2 can be substituted with alanine (A), serine (S) or arginine (R). In a particular aspect, the amino acid corresponding to Y102 of SEQ ID NO: 2 can be substituted with alanine. In a particular aspect the J-chain or functional fragment or variant thereof is a variant human J-chain and comprises the amino acid sequence SEQ ID NO: 3, a J chain referred to herein as “J*”.

Wild-type J-chains typically include one N-linked glycosylation site. In certain embodiments, a variant J-chain or functional fragment thereof of a multimeric binding molecule as provided herein includes a mutation within the asparagine (N)-linked glycosylation motif N-X₁-S/T, e.g., starting at the amino acid position corresponding to amino acid 49 (motif N6) of the mature human J-chain (SEQ ID NO: 2) or J* (SEQ ID NO: 3), where N is asparagine, X₁ is any amino acid except proline, and S/T is serine or threonine, and where the mutation prevents glycosylation at that motif. For example, in some embodiments, the J-chain comprises a substitution at N49, such as N49D. In some embodiments, the J-chain comprises amino acids 1-137 of SEQ ID NO: 86. As demonstrated in PCT Publication No. WO 2019/169314, mutations preventing glycosylation at N49 can result in the multimeric binding molecule as provided herein, exhibiting an increased serum half-life upon administration to a subject animal relative to a reference multimeric binding molecule that is identical except for the mutation or mutations preventing glycosylation in the variant J-chain, and is administered in the same way to the same animal species.

For example, in certain embodiments the variant J-chain or functional fragment or variant thereof of a binding molecule comprising a J-chain as provided herein can include an amino acid substitution at the amino acid position corresponding to amino acid N49 or amino acid S51 of SEQ ID NO: 2 or SEQ ID NO: 3, provided that the amino acid corresponding to S51 is not substituted with threonine (T), or where the variant J-chain comprises amino acid substitutions at the amino acid positions corresponding to both amino acids N49 and S51 of SEQ ID NO: 2 or SEQ ID NO: 3. In certain embodiments, the position corresponding to N49 of SEQ ID NO: 2 or SEQ ID NO: 3 is substituted with any amino acid, e.g., alanine (A), glycine (G), threonine (T), serine (S) or aspartic acid (D). In a particular embodiment, the position corresponding to N49 of SEQ ID NO: 2 or SEQ ID NO: 3 can be substituted with alanine (A). In another particular embodiment, the position corresponding to N49 of SEQ ID NO: 2 or SEQ ID NO: 3 can be substituted with aspartic acid (D). In some embodiments, the position corresponding to S51 of SEQ ID NO: 2 or SEQ ID NO: 3 is substituted with alanine (A) or glycine (G). In some embodiments, the position corresponding to S51 of SEQ ID NO: 2 or SEQ ID NO: 3 is substituted with alanine (A).

Multimeric Binding Molecules with a Modified J-Chain Expressing an Immune Stimulatory Agent

This disclosure provides multimeric binding molecules with immune stimulatory properties. In certain aspects, the disclosure provides a multimeric binding molecule that includes two IgA or IgA-like bivalent binding units or five IgM or IgM-like bivalent binding units or multimerizing variants or fragments thereof and a modified J-chain. Each binding unit includes either two IgA or two IgM heavy chain constant regions or multimerizing variants or fragments thereof, each associated with an antigen-binding domain for a total of four or ten antigen-binding domains, which can be the same or different, but in certain aspects at least three, at least four, at least five, at least six, at least seven, at least eight, at least nine or ten of the antigen-binding domains of the binding molecule specifically bind to a target antigen. The antigen binding domains can be identical, or can be different, e.g., binding to different epitopes of the same target antigen.

The modified J-chain of the provided multimeric binding molecule includes (a) a J-chain or functional fragment or variant thereof (“J”), and (b) an immunostimulatory agent (“ISA”), wherein J and the ISA are associated as a fusion protein. As used herein, the term “ISA” can refer to the heterologous moiety fused to the J-chain that possesses immune stimulatory activity, or can refer to entire multimeric binding molecule, which possesses immune stimulatory activity. In certain aspects the ISA comprises a cytokine, or a receptor-binding fragment or variant thereof. For example, the ISA can include interleukin-15 (IL-15), interleukin-2 (IL-2), interferon (IFN)-α, interleukin 12 (IL-12), interleukin-21 (IL-21), granulocyte macrophage colony-stimulating factor (GM-CSF), or any receptor-binding fragment or variant thereof. As described below, the ISA can, in addition, include portions of a receptor subunit, or other immune stimulating moieties.

IL-15, complexed with the sushi domain of IL-15Rα, forms a highly potent ISA that can stimulate CD8+ T cells and NK cells. Accordingly, in certain aspects the disclosure provides a modified J-chain comprising a J-chain or functional fragment or variant thereof (“J”), and (a) an interleukin-15 (IL-15) protein or receptor-binding fragment or variant thereof (“I”), and/or (b) an interleukin-15 receptor-α (IL-15Rα) fragment comprising the sushi domain or a variant thereof capable of associating with I (“R”), wherein J and at least one of I and R are associated as a fusion protein, and wherein I and R can associate to function as the ISA. “J” can be a wild-type J-chain of any species, e.g., a wild-type human J-chain comprising the amino acid sequence SEQ ID NO: 2 or a functional fragment or variant thereof.

Alternatively, “J” can be a variant J-chain or fragment thereof comprising one or more single amino acid substitutions, deletions, or insertions relative to a wild-type J-chain that can affect, e.g., the serum half-life of the multimeric binding molecule comprising the J-chain, as described in PCT Publication No. WO 2019/169314A1. In certain aspects, “J” is a variant human J-chain and comprises the amino acid sequence SEQ ID NO: 3, also referred to herein as (“J*”).

In certain aspects, the interleukin-15 (IL-15) protein or receptor-binding fragment or variant thereof (“I”) of the immune stimulatory agent is a wild-type human IL-15 protein comprising the amino acid sequence SEQ ID NO: 4. Non-limiting examples of modified J-chain ISAs comprising the wild-type human IL-15 are provided herein e.g., SEQ ID NO: 6, SEQ ID NO: 19, SEQ ID NO: 20, SEQ ID NO: 21, SEQ ID NO: 22, SEQ ID NO: 23, SEQ ID NO: 24, SEQ ID NO: 25, or SEQ ID NO: 26.

Multimeric binding molecules comprising an immune stimulatory agent (ISA) as provided herein can efficiently stimulate proliferation and activation of immune effector cells, e.g., CD8+ cytotoxic T lymphocytes or natural killer (NK) cells. Accordingly, multimeric binding molecules comprising an ISA as provided herein can function as effective therapeutics to treat, e.g., cancer or infectious diseases. In certain contexts, however, it can be desirable to modulate, e.g., reduce the potency of the effector cell stimulation to allow for sufficient effector cell proliferation while minimizing toxic side effects such as cytokine release syndrome (CRS). Accordingly, this disclosure provides multimeric binding molecules in which the potency of the ISA activity is modulated, e.g., altered or reduced. In certain aspects, “I” comprises a receptor binding variant of human IL-15, in which receptor binding is reduced but not eliminated. In certain aspects the receptor binding variant of human IL-15 comprises at least one, at least two, at least three, at least four, at least five, at least six, at least seven, at least eight, or at least nine single amino acid insertions, deletions, or substitutions, where the single amino acid insertions, deletions, or substitutions reduce the affinity of the IL-15 variant for its receptor. Variant versions of human IL-15 that achieve this goal are described in PCT Publication No. WO 2018/071918A1. In certain aspects the variant human IL-15 comprises at least one, at least two, at least three, at least four, at least five, at least six, at least seven, at least eight, or at least nine single amino acid insertions, deletions, or substitutions, but no more than ten, single amino acid insertions, deletions, or substitutions. In certain aspects I comprises a variant human IL-15 comprising one, two, three, four, five, six, seven, eight or nine single amino acid substitutions. In certain aspects the amino acid substitutions are at one or more of positions corresponding to N1, N4, D8, D30, D61, E64, N65, N72, or Q108 of SEQ ID NO: 4. In certain aspects, the amino acid substitutions comprise one or more of substitutions N1D, N4D, D8N, D30N, D61N, E64Q, N65D, N72D, or Q108E, in SEQ ID NO: 4. For example, in certain multimeric binding molecules provided herein I comprises SEQ ID NO: 4 except for: a single amino acid substitution at a position selected from the group consisting of N1D, N4D, D8N, D30N, D61N, E64Q, N65D, N72D, and Q108E; two amino acid substitutions at positions selected from the group consisting of N4D/N65D and N1D/N65D; or three amino acid substitutions at positions D30N/E64Q/N65D. In certain aspects I comprises the amino acid sequence SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, or SEQ ID NO: 68. In certain aspects, the J*RI ISA comprises the amino acid sequence SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, or SEQ ID NO: 18.

As shown in the Examples, anti-PD-L1 IgM pentamers comprising a modified J-chain ISA in the J*RI configuration and comprising one, two, or three of these mutations can still trigger proliferation of CD8+ T cells and NK cells, but at reduced potency relative to a corresponding ISA comprising the wild-type human IL-15.

In certain aspects, “R” comprises the sushi domain of the human IL-15 receptor-α. In certain aspects, R comprises the amino acid sequence SEQ ID NO: 5 or a variant or fragment thereof that is capable of associating with human IL-15. In certain aspects R consists essentially of or consists of the amino acid sequence SEQ ID NO: 5 or a variant thereof that is capable of associating with human IL-15.

A modified J-chain ISA comprising IL-15 and the sushi domain of the IL-15Rα can be configured in a number of ways. Typically, at least I or R is associated with J as a fusion protein. For example, where J is 1*, the configuration can be J*I, IJ*, J*R, or RJ*. In these embodiments, I or R can be provided as a separate protein subunit that can associate with R or I fused to 1*. In certain aspects, both I and R are fused to the J-chain. For example, where J is J*, the configuration can be J*RI, J*IR, RIP*, IRJ*, IJ*R, or RJ*I. Typically, heterologous moieties are fused to the J-chain or variant or fragment thereof via a linker, a small, flexible chain of amino acids, typically comprising small amino acids, e.g., glycine (G) and/or serine (S). Exemplary linkers comprise (GGGGS)n, where n is an integer from 1 to 10 (SEQ ID NO: 226). For example the linker can comprise, consist of, or consist essentially of SEQ ID NO: 80, SEQ ID NO: 81, SEQ ID NO: 82, or SEQ ID NO: 83. Where the J-chain, e.g., J or J* is fused to both I and R, linkers are typically employed between each element for a total of at least two linkers. The at least two linkers can be the same or different. In certain aspects at least one linker comprises, consists essentially of, or consists of the amino acid sequence GGGGSGGGGSGGGGS (SEQ ID NO: 78). In certain aspects at least one linker comprises, consists essentially of, or consists of the amino acid sequence GGSGGGGSGGGSGGGGSLQ (SEQ ID NO: 79). In certain aspects, a multimeric binding molecule comprising a modified J-chain with immune stimulatory activity, where the modified J chain comprises the J* mutation, and where the modified J-chain is arranged from N-terminus to C-terminus as J*-R-I, J*-I-R, I-R-J*, R-I-J, R-J*-I, I-J*-R, I-J*, or J*-I, wherein “-” is a linker. In certain aspect, the modified J-chain of the multimeric binding molecule comprises the amino acid sequence SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 20, SEQ ID NO: 21, SEQ ID NO: 22, SEQ ID NO: 23, SEQ ID NO: 24, SEQ ID NO: 25, or SEQ ID NO: 26.

In certain aspects, the modified J-chain is arranged from N-terminus to C-terminus as J*-R-I. Exemplary ISAs in this configuration for inclusion in a multimeric binding molecule as provided herein comprise the amino acid sequence SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, or SEQ ID NO: 18.

The modified J-chain of the provided multimeric binding molecule includes (a) a J-chain or functional fragment or variant thereof (“J”), and (b) an immunostimulatory agent (“ISA”), wherein J and the ISA are associated as a fusion protein. In certain aspects, the ISA can comprise the cytokine IL-2, or a variant thereof. Wild-type human IL-2, when used as cancer immunotherapy, can cause severe side effects in humans. Accordingly, variants of IL-2 have been developed that bind to the lower affinity dimeric β/γ receptor but not to the high affinity trimeric α/β/γ receptor. Accordingly, the variants exhibit lower potency and lower levels of toxic side effects. One variant IL-2v, is described in U.S. Pat. No. 9,266,938, and is presented herein as SEQ ID NO: 31. A modified J-chain ISA comprising IL-2v is presented herein as SEQ ID NO: 32.

In certain aspects a modified J-chain of a multimeric binding molecule as provided herein can further comprise, in addition to an ISA, an antigen-binding domain of an antibody fused thereto. For example, a modified J-chain such as J*RI provided herein can further include a single-chain Fv binding domain fused to the N-terminus of the variant J-chain. Such an antigen binding domain can be used to target immune effector cells such as cytotoxic T lymphocytes (CTLs) or NK cells which can then be stimulated to proliferate in response to the ISA. Thus, in certain aspects a modified J-chain as provided herein can further comprise a scFv antigen-binding domain that binds to a target on an immune effector cell, e.g., CTLs or NK cells. Where it is desired to target NK cells, the scFv can specifically target CD16. Where it is desired to target CD8+ cytotoxic T cells, the scFv can specifically target CD3, e.g., CD3ε. An exemplary modified J-chain is provided that comprises S-J*-R-I, where S is a scFv comprising the VH and VL regions of mouse anti-human CD3 monoclonal antibody SP34, J* is a human J chain variant comprising a Y102A mutation in the human J-chain sequence, I is human IL-15, and R is the sushi domain of the human IL-15 receptor-α, where each “-” comprises a linker. In certain aspects the modified J-chain in this configuration comprises the amino acid sequence SEQ ID NO: 19. Other CD3R antigen-binding domains can also be utilized, including, but not limited to the VH and VL of visilizumab, OKT3, or the CD3 binders disclosed in PCT Publication No. WO 2018/208864.

Other ISAs comprising cytokine or cytokine variants fused to a modified J-chain are provided and will be easily contemplated by the skilled person based on the present disclosure.

Antigen-Binding Domains

A multimeric binding molecule as provided herein includes at least one, at least two, at least three, at least four, at least five, at least six, at least seven, at least eight, at least nine, at least ten, at least eleven, or twelve heavy chain constant regions associated with a binding domain, e.g., an antigen-binding domain, that specifically binds to a target of interest. In certain aspects, the target is a target epitope, a target antigen, a target cell, a target organ, or a target virus. Targets can include, without limitation, tumor antigens, other oncologic targets, immuno-oncologic targets such as immune checkpoint inhibitors, infectious disease antigens, such as viral antigens expressed on the surface of infected cells, target antigens involved in blood-brain-barrier transport, target antigens involved in neurodegenerative diseases and neuroinflammatory diseases, and any combination thereof. Exemplary targets and binding domains that bind to such targets are provided elsewhere herein, and can be found in, e.g., U.S. Patent Application Publication Nos. US-2019-0100597, or US-2019-0185570, in PCT Publication Nos. WO/2017/196867, WO 2018/017888, WO 2018/017889, WO 2018/017761, WO 2018/017763, or WO 2018/187702, WO 2019/165340, WO 2019/169314A1, WO 2020/086745A1, or in U.S. Pat. Nos. 9,951,134, 9,938,347, 8,377,435, 9,458,241, 9,409,976, 10,351,631, 10,570,191, 10,604,559 or 10,618,978. Each of these applications and/or patents is incorporated herein by reference in its entirety.

In certain aspects a multimeric binding molecule as provided herein comprises at least three, at least four, at least five, at least six, at least seven, at least eight, at least nine, antigen binding domains that specifically bind to a target antigen, where the target antigen comprises a tumor-specific antigen, a tumor-associated antigen, or a target that modulates a T cell response or NK cell response.

In certain aspects the antigen binding domains bind to a target that modulates a T cell response or an NK cell response. For example, certain targets in their normal activity can promote tumor growth by inhibiting cytotoxic CD8+ T cell or NK cell activity, antigen binding domains that antagonize these targets can promote CD8+ or NK cell activity. Such targets include, without limitation inhibitory immune checkpoint proteins. In certain aspects, the inhibitory immune checkpoint protein comprises a programmed cell death-1 protein (PD-1), a programmed cell death ligand-1 protein (PD-L1), a lymphocyte-activation gene 3 protein (LAG3), a T-cell immunoglobulin and mucin domain 3 protein (TIM3), a cytotoxic T-lymphocyte-associated protein 4 (CTLA4), a B- and T-lymphocyte attenuator protein (BTLA), a V-domain Ig suppressor of T-cell activation protein (VISTA), a T-cell immunoreceptor with Ig and ITIM Domains protein (TIGIT), a Killer-cell Immunoglobulin-like Receptor protein (KIR), a B7-H3 protein, a B7-H4 protein, or any combination thereof, and the antigen binding domains of the multimeric binding molecule provided herein antagonize the targets, thereby promoting immune effector cell activity.

In certain aspects the inhibitory immune checkpoint protein comprises PD-L1. This disclosure contemplates any antigen binding domains that specifically bind to and inhibit PD-L1, including antibodies currently in the clinic or commercially available such as Pembrolizumab, Nivolumab, Atezolizumab, or Durvalumab. In certain aspects and wherein the antigen-binding domain comprises the heavy chain variable region (VH) and the light chain variable region (VL) of the humanized anti-PD-L1 antibody h3C5, disclosed in U.S. Patent Application Publication No. 2019/0338031, which is incorporated herein by reference. The VH comprises the amino acid sequence SEQ ID NO: 33, SEQ ID NO: 91, SEQ ID NO: 92, or SEQ ID NO: 93 and the VL comprises the amino acid sequence SEQ ID NO: 34 or SEQ ID NO: 94. Alternatively the PD-L1 antibody can comprise the VH and VL of the phage library-derived anti-PD-L1 antibody YW243.55.S70 as disclosed in U.S. Pat. No. 8,217,149, the antigen binding domain comprising the VH amino acid sequence SEQ ID NO: 75 and the VL amino acid sequence SEQ ID NO: 76. Alternatively, the PD-L1 antibody can comprise the CDRs with zero, one, or two substitutions, or VH and VL sequences with 85%, 90%, 95%, 99%, or 100% sequence identity to the VH and VL sequences of SEQ ID NO: 96 and SEQ ID NO: 97, SEQ ID NO: 98 and SEQ ID NO: 99, SEQ ID NO: 100 and SEQ ID NO: 101, SEQ ID NO: 102 and SEQ ID NO: 103, SEQ ID NO: 104 and SEQ ID NO: 105, SEQ ID NO: 106 and SEQ ID NO: 107, SEQ ID NO: 108 and SEQ ID NO: 109, SEQ ID NO: 110 and SEQ ID NO: 111, SEQ ID NO: 112 and SEQ ID NO: 113, SEQ ID NO: 114 and SEQ ID NO: 115, SEQ ID NO: 116 and SEQ ID NO: 117, SEQ ID NO: 118 and SEQ ID NO: 119, SEQ ID NO: 120 and SEQ ID NO: 121, SEQ ID NO: 122 and SEQ ID NO: 123, SEQ ID NO: 124 and SEQ ID NO: 125, SEQ ID NO: 126 and SEQ ID NO: 127, SEQ ID NO: 128 and SEQ ID NO: 129, SEQ ID NO: 130 and SEQ ID NO: 131, SEQ ID NO: 132 and SEQ ID NO: 133, SEQ ID NO: 134 and SEQ ID NO: 135, SEQ ID NO: 136 and SEQ ID NO: 137, SEQ ID NO: 138 and SEQ ID NO: 139, SEQ ID NO: 140 and SEQ ID NO: 141, SEQ ID NO: 142 and SEQ ID NO: 143, SEQ ID NO: 144 and SEQ ID NO: 145, SEQ ID NO: 146 and SEQ ID NO: 147, SEQ ID NO: 148 and SEQ ID NO: 149, SEQ ID NO: 150 and SEQ ID NO: 151, SEQ ID NO: 152 and SEQ ID NO: 153, SEQ ID NO: 154 and SEQ ID NO: 155, SEQ ID NO: 156 and SEQ ID NO: 157, SEQ ID NO: 158 and SEQ ID NO: 159, SEQ ID NO: 160 and SEQ ID NO: 161, SEQ ID NO: 162 and SEQ ID NO: 163, SEQ ID NO: 164 and SEQ ID NO: 165, SEQ ID NO: 166 and SEQ ID NO: 167, SEQ ID NO: 168 and SEQ ID NO: 169, SEQ ID NO: 170 and SEQ ID NO: 171, SEQ ID NO: 172 and SEQ ID NO: 173, SEQ ID NO: 174 and SEQ ID NO: 175, SEQ ID NO: 176 and SEQ ID NO: 177, SEQ ID NO: 178 and SEQ ID NO: 179, SEQ ID NO: 180 and SEQ ID NO: 181, SEQ ID NO: 182 and SEQ ID NO: 183, SEQ ID NO: 184 and SEQ ID NO: 185, SEQ ID NO: 186 and SEQ ID NO: 187, SEQ ID NO: 188 and SEQ ID NO: 189, SEQ ID NO: 190 and SEQ ID NO: 191, SEQ ID NO: 192 and SEQ ID NO: 193, SEQ ID NO: 194 and SEQ ID NO: 195, SEQ ID NO: 196 and SEQ ID NO: 197, SEQ ID NO: 198 and SEQ ID NO: 199, SEQ ID NO: 200 and SEQ ID NO: 201, SEQ ID NO: 202 and SEQ ID NO: 203, SEQ ID NO: 204 and SEQ ID NO: 205, SEQ ID NO: 206 and SEQ ID NO: 207, SEQ ID NO: 208 and SEQ ID NO: 209, SEQ ID NO: 210 and SEQ ID NO: 211, SEQ ID NO: 212 and SEQ ID NO: 213, SEQ ID NO: 214 and SEQ ID NO: 215, SEQ ID NO: 216 and SEQ ID NO: 217, SEQ ID NO: 218 and SEQ ID NO: 219, SEQ ID NO: 220 and SEQ ID NO: 221, or SEQ ID NO: 222 and SEQ ID NO: 223, respectively. In some embodiments, the PD-L1 antibody can comprise the CDRs with zero, one, or two substitutions, or VH and VL sequences with 85%, 90%, 95%, 99%, or 100% sequence identity to the VH and VL sequences of SEQ ID NO: 134 and SEQ ID NO: 135, SEQ ID NO: 136 and SEQ ID NO: 137, SEQ ID NO: 138 and SEQ ID NO: 139, SEQ ID NO: 140 and SEQ ID NO: 141, SEQ ID NO: 142 and SEQ ID NO: 143, SEQ ID NO: 144 and SEQ ID NO: 145, SEQ ID NO: 146 and SEQ ID NO: 147, SEQ ID NO: 148 and SEQ ID NO: 149, SEQ ID NO: 166 and SEQ ID NO: 167, SEQ ID NO: 168 and SEQ ID NO: 169, SEQ ID NO: 170 and SEQ ID NO: 171, or SEQ ID NO: 186 and SEQ ID NO: 187.

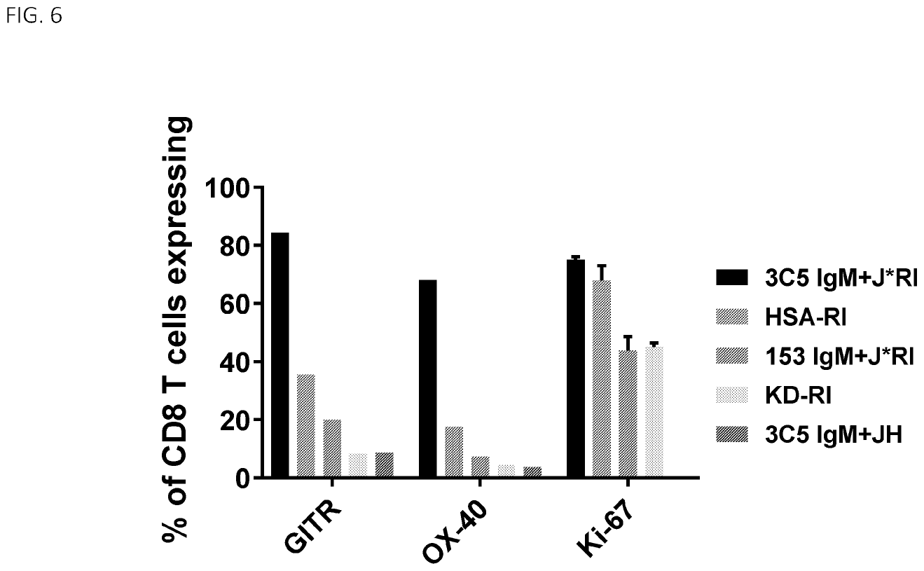

In certain aspects the target is one that enhances immune effector cell activity, e.g., CD8+ T cell or NK cell activity, and the antigen binding domains of the multimeric binding molecule provided herein agonizes the target, thereby stimulating immune effector activity. For example, in certain aspects the target comprises a TNF receptor superfamily target that acts on immune effector cells, and wherein the antigen-binding domains can agonize the target. Exemplary TNFrSF targets in this category include Glucocorticoid-induced TNFR-related protein (GITR) and OX40. Expression of both of these targets is upregulated by certain ISAs provided herein. See, e.g., FIG. 6.

GITR is an activating receptor that is expressed on the surface of T cells and other immune cells. Once exposure to tumor antigen activates a T cell, the number of GITR receptors on its surface increases. GITR acts as a costimulatory receptor on activated T cells and enhances CD8+ T cell proliferation. Signaling through GITR also inhibits regulatory T cells. Multimeric agonist antibodies targeting GITR as disclosed, e.g., in U.S. Patent Application Publication No. 2019/0330360A1 and in PCT Application No.: PCT/US2020/017083, which are incorporated herein by reference in their entireties. In certain aspects, the GITR antigen binding domains can be any anti-GITR agonist antibody, including, but not limited to those listed in U.S. Patent Application Publication No. 2019/0330360A1. In certain aspects the anti-GITR antigen-binding domain comprises a heavy chain variable region (VH) and a light chain variable region (VL) comprising, respectively, the amino acid sequences SEQ ID NO: 35 and SEQ ID NO: 36, SEQ ID NO: 37 and SEQ ID NO: 38, SEQ ID NO: 39 and SEQ ID NO: 40, SEQ ID NO: 41 and SEQ ID NO: 42, or SEQ ID NO: 43 and SEQ ID NO: 44.

OX40 is an activating receptor expressed on the surface of activated cytotoxic T cells and regulatory T cells (Tregs). Signaling through OX40 plays a dual role in the immune response, both activating and amplifying T-cell responses. Cytotoxic T cells are able to recognize and attack tumor cells. On cytotoxic T cells, OX40 binds to its ligand (OX40L), resulting in stimulatory signals that promote T-cell reproduction, function, and survival. Tregs act to limit the immune response. OX40-OX40L signaling blocks the ability of Tregs to suppress T cells and reduces Treg generation. By inhibiting the immunosuppressive effect of Tregs and limiting their population, OX40 further amplifies the impact of T-cell activation. Multimeric agonist antibodies targeting OX40 as disclosed, e.g., in U.S. Patent Application Publication No. 2019/0330374, which is incorporated herein by reference in its entirety. In certain aspects, the OX40 antigen binding domains can be any anti-OX40 agonist antibody, including, but not limited to those listed in U.S. Patent Application Publication No. 2019/0330374. In certain aspects the antigen-binding domain comprises a heavy chain variable region (VH) and a light chain variable region (VL) comprising, respectively, the amino acid sequences SEQ ID NO: 45 and SEQ ID NO: 46 or SEQ ID NO: 47 and SEQ ID NO: 48.

In certain aspects the target is a tumor-specific antigen, i.e., a target antigen that is largely or primarily expressed only on tumor or cancer cells, or that may be expressed only at reduced or undetectable levels in normal healthy cells of an adult. In certain aspects the target is a tumor-associated antigen, i.e., a target antigen that is expressed on both healthy and cancerous cells but is expressed at much higher density on cancerous cells than on normal healthy cells. Exemplary tumor-specific and tumor-associated antigens include, without limitation, B-cell maturation antigen (BCMA), CD19, CD20, epidermal growth factor receptor (EGFR), human epidermal growth factor receptor 2 (HER2, also called ErbB2), HER3 (ErbB3), receptor tyrosine-protein kinase ErbB4, cytotoxic T-lymphocyte antigen 4 (CTLA4), programmed cell death protein 1 (PD-1), Programmed death-ligand 1 (PD-L1), vascular endothelial growth factor (VEGF), VEGF receptor-1 (VEGFR1), VEGFR2, CD52, CD30, prostate-specific membrane antigen (PSMA), CD38, ganglioside GD2, self-ligand receptor of the signaling lymphocytic activation molecule family member 7 (SLAMF7), platelet-derived growth factor receptor A (PDGFRA), CD22, FLT3 (CD135), CD123, MUC-16, carcinoembryonic antigen-related cell adhesion molecule 1 (CEACAM-1), mesothelin, tumor-associated calcium signal transducer 2 (Trop-2), glypican-3 (GPC-3), human blood group H type 1 trisaccharide (Globo-H), sialyl Tn antigen (STn antigen), or CD33. The skilled person will understand that these target antigens appear in the literature by a number of different names, but that these well-known therapeutic targets can be easily identified using databases available online, e.g., EXPASY.org.

Other tumor associated and/or tumor-specific antigens include, without limitation: DLL4, Notch1, Notch2, Notch3, Notch4, JAG1, JAG2, c-Met, IGF-1R, Patched, Hedgehog family polypeptides, WNT family polypeptides, FZD1, FZD2, FZD3, FZD4, FZD5, FZD6, FZD7, FZD8, FZD9, FZD10, LRP5, LRP6, IL-6, TNFalpha, IL-23, IL-17, CD80, CD86, CD3, CEA, Muc16, PSCA, CD44, c-Kit, DDR1, DDR2, RSPO1, RSPO2, RSPO3, RSPO4, BMP family polypeptides, BMPR1a, BMPR1b, or a TNF receptor superfamily protein such as TNFR1 (DR1), TNFR2, TNFR1/2, CD40 (p50), Fas (CD95, Apo1, DR2), CD30, 4-1BB (CD137, ILA), TRAILR1 (DR4, Apo2), DR5 (TRAILR2), TRAILR3 (DcR1), TRAILR4 (DcR2), OPG (OCIF), TWEAKR (FN14), LIGHTR (HVEM), DcR3, DR3, EDAR, or XEDAR.

The multimeric binding molecule of claim 39, wherein the target antigen comprises a tumor-associated antigen.

The multimeric binding molecule of claim 51, wherein the tumor associated antigen comprises B-cell maturation antigen (BCMA), CD19, CD20, EGFR, HER2 (ErbB2), ErbB3, ErbB4, CTLA4, PD-1, PD-L1, VEGF, VEGFR1, VEGFR2, CD52, CD30, prostate-specific membrane antigen (PSMA), CD38, GD2, SLAMF7, platelet-derived growth factor receptor A (PDGFRA), CD22, FLT3 (CD135), CD123, MUC-16, carcinoembryonic antigen-related cell adhesion molecule 1 (CEACAM-1), mesothelin, tumor-associated calcium signal transducer 2 (Trop-2), glypican-3 (GPC-3), human blood group H type 1 trisaccharide (Globo-H), sialyl Tn antigen (STn antigen), CD33, or any combination thereof.

In certain aspects the target antigen comprises CD20, Any CD20 antigen binding domains can be used. An exemplary antigen-binding domain comprises a heavy chain variable region (VH) and a light chain variable region (VL) comprising, respectively, the amino acid sequences SEQ ID NO: 49 and SEQ ID NO: 50.

In certain aspects, at least four, at least five, at least six, at least seven, at least eight, at least nine or ten of the antigen-binding domains of the binding molecule specifically bind to the same target antigen. In certain aspects, the at least four, at least five, at least six, at least seven, at least eight, at least nine or ten antigen-binding domains are identical.

Polynucleotides, Vectors, and Host Cells

The disclosure further provides a polynucleotide, e.g., an isolated, recombinant, and/or non-naturally occurring polynucleotide, comprising a nucleic acid sequence that encodes a polypeptide subunit of a multimeric binding molecule as provided herein. By “polypeptide subunit” is meant a portion of a binding molecule, binding unit, IgM antibody, IgM-like antibody, IgA antibody, or IgA-like antibody or antigen-binding domain that can be independently translated. Examples include, without limitation, an antibody variable domain, e.g., a VH or a VL, a J chain, including modified J-chains as provided herein, a secretory component, a single chain Fv, an antibody heavy chain, an antibody light chain, an antibody heavy chain constant region, an antibody light chain constant region, and/or any fragment, variant, or derivative thereof.

In certain aspects, the disclosure provides an isolated polynucleotide comprising a nucleic acid encoding a subunit polypeptide of any multimeric binding molecule provided herein, wherein the subunit polypeptide comprises (a) an IgA or IgM heavy chain comprising an IgA or IgM heavy chain constant region or a multimerizing variant or fragment thereof associated with an antibody heavy chain variable region (VH), (b) an antibody light chain comprising an antibody light chain constant region associated with an antibody light chain variable region (VL), or (c) a modified J-chain comprising two or more of (i) a J-chain or functional fragment or variant thereof (“J”), (ii) an interleukin-15 (IL-15) protein or receptor-binding fragment or variant thereof (“I”), or (iii) an interleukin-15 receptor-α (IL-15Rα) fragment comprising the sushi domain or a variant thereof capable of associating with I (“R”), or (iv) an interleukin-2v protein (IL-2v), wherein J and at least one of I, R, or IL-2v are associated as a fusion protein, and wherein I and R can associate to function as an immune stimulatory complex, or (d) any combination thereof.

In certain aspects, the polypeptide subunit can comprise an IgM heavy chain constant region or IgM-like heavy chain constant region or multimerizing fragment thereof, or an IgA heavy chain constant region or IgA-like heavy chain constant region or multimerizing fragment thereof, which can be fused to an antigen-binding domain or a subunit thereof, e.g., to the VH portion of an antigen-binding domain, all as provided herein. In certain aspects the polynucleotide can encode a polypeptide subunit comprising a human IgM heavy chain constant region, a human IgM-like heavy chain constant region, a human IgA heavy chain constant region, a human IgA-like heavy chain constant region, or multimerizing fragment thereof, e.g., SEQ ID NO: 51, SEQ ID NO: 52, SEQ ID NO: 53, or SEQ ID NO: 54, any of which can be fused to an antigen-binding domain or subunit thereof, e.g., the C-terminal end of a VH.

In certain aspects, the polypeptide subunit can comprise an antibody VL portion of an antigen-binding domain as described elsewhere herein. In certain aspects the polypeptide subunit can comprise an antibody light chain constant region, e.g., a human antibody light chain constant region, or fragment thereof, which can be fused to the C-terminal end of a VL.

In certain aspects the polypeptide subunit can comprise a J-chain, a modified J-chain, or any functional fragment or variant thereof, as provided herein. In certain aspects the polypeptide subunit can comprise a human J-chain or functional fragment or variant thereof, including any modified J-chains. In certain aspects the J-chain can comprise the amino acid sequence SEQ ID NO: 1, SEQ ID NO: 2, SEQ ID NO: 3, SEQ ID NO: 6, SEQ ID NO: 7, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15 SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, or SEQ ID NO: 32.

In certain aspects a polynucleotide as provided herein, e.g., an expression vector such as a plasmid, can include a nucleic acid sequence encoding one polypeptide subunit, e.g., an IgM heavy chain or IgM-like heavy chain, a light chain, or a J-chain, or can include two or more nucleic acid sequences encoding two or more or all three polypeptide subunits of an IgM antibody or IgM-like antibody as provided herein. Alternatively, the nucleic acid sequences encoding the three polypeptide subunits can be on separate polynucleotides, e.g., separate expression vectors. The disclosure provides such single or multiple expression vectors. The disclosure also provides one or more host cells encoding the provided polynucleotide(s) or expression vector(s).

The disclosure further provides a composition comprising two or more polynucleotides, where the two or more polynucleotides collectively can encode multimeric binding molecule as provided herein.

The disclosure further provides a host cell, e.g., a prokaryotic or eukaryotic host cell, comprising a polynucleotide or two or more polynucleotides encoding a multimeric binding molecule as provided herein, or any subunit thereof, a polynucleotide composition as provided herein, or a vector or two, three, or more vectors that collectively encode the IgM or IgM-like antibody as provided herein, or any subunit thereof.

In a related aspect, the disclosure provides a method of producing a multimeric binding molecule as provided by this disclosure, where the method comprises culturing a host cell as provided herein and recovering the multimeric binding molecule.

Methods of Use

The disclosure further provides a method of treating a disease or disorder in a subject in need of treatment, comprising administering to the subject a therapeutically effective amount of a multimeric binding molecule comprising an ISA as provided herein. By “therapeutically effective dose or amount” or “effective amount” is intended an amount of the multimeric binding molecule that when administered brings about a positive immunotherapeutic response with respect to treatment of subject.

Effective doses of compositions for treatment of cancer vary depending upon many different factors, including means of administration, target site, physiological state of the subject, whether the subject is human or an animal, other medications administered, and whether treatment is prophylactic or therapeutic. Usually, the subject is a human, but non-human mammals including transgenic mammals can also be treated. Treatment dosages can be titrated using routine methods known to those of skill in the art to optimize safety and efficacy.

The subject to be treated can be any animal, e.g., mammal, in need of treatment, in certain aspects, the subject is a human subject.

In its simplest form, a preparation to be administered to a subject is the multimeric binding molecule comprising an ISA as provided herein, or a multimeric antigen-binding fragment thereof, administered in conventional dosage form, which can be combined with a pharmaceutical excipient, carrier or diluent as described elsewhere herein.

The compositions of the disclosure can be administered by any suitable method, e.g., parenterally, intraventricularly, orally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques.

Pharmaceutical Compositions and Administration Methods

Methods of preparing and administering a multimeric binding molecule comprising an ISA as provided herein to a subject in need thereof are well known to or are readily determined by those skilled in the art in view of this disclosure. The route of administration of can be, for example, intratumoral, oral, parenteral, by inhalation or topical. The term parenteral as used herein includes, e.g., intravenous, intraarterial, intraperitoneal, intramuscular, subcutaneous, rectal, or vaginal administration. While these forms of administration are contemplated as suitable forms, another example of a form for administration would be a solution for injection, in particular for intratumoral, intravenous, or intraarterial injection or drip. A suitable pharmaceutical composition can comprise a buffer (e.g. acetate, phosphate or citrate buffer), a surfactant (e.g. polysorbate), optionally a stabilizer agent (e.g. human albumin), etc.

As discussed herein, a multimeric binding molecule comprising an ISA as provided herein can be administered in a pharmaceutically effective amount for the treatment of a subject in need thereof. In this regard, it will be appreciated that the disclosed multimeric binding molecule comprising an ISA can be formulated so as to facilitate administration and promote stability of the active agent. Pharmaceutical compositions accordingly can comprise a pharmaceutically acceptable, non-toxic, sterile carrier such as physiological saline, non-toxic buffers, preservatives, and the like. A pharmaceutically effective amount of a multimeric binding molecule comprising an ISA as provided herein means an amount sufficient to achieve effective binding to a target and to achieve a therapeutic benefit. Suitable formulations are described in Remington's Pharmaceutical Sciences, e.g., 21^{st} Edition (Lippincott Williams & Wilkins) (2005).

Certain pharmaceutical compositions provided herein can be orally administered in an acceptable dosage form including, e.g., capsules, tablets, aqueous suspensions or solutions. Certain pharmaceutical compositions also can be administered by nasal aerosol or inhalation. Such compositions can be prepared as solutions in saline, employing benzyl alcohol or other suitable preservatives, absorption promoters to enhance bioavailability, and/or other conventional solubilizing or dispersing agents.

The amount of a multimeric binding molecule comprising an ISA that can be combined with carrier materials to produce a single dosage form will vary depending, e.g., upon the subject treated and the particular mode of administration. The composition can be administered as a single dose, multiple doses or over an established period of time in an infusion. Dosage regimens also can be adjusted to provide the optimum desired response (e.g., a therapeutic or prophylactic response).

In keeping with the scope of the present disclosure, a multimeric binding molecule comprising an ISA as provided herein can be administered to a subject in need of therapy in an amount sufficient to produce a therapeutic effect. A multimeric binding molecule comprising an ISA as provided herein can be administered to the subject in a conventional dosage form prepared by combining the antibody or multimeric antigen-binding fragment, variant, or derivative thereof of the disclosure with a conventional pharmaceutically acceptable carrier or diluent according to known techniques. The form and character of the pharmaceutically acceptable carrier or diluent can be dictated by the amount of active ingredient with which it is to be combined, the route of administration and other well-known variables.

This disclosure also provides for the use of a multimeric binding molecule comprising an ISA as provided herein in the manufacture of a medicament for treating, preventing, or managing cancer. The disclosure also provides for a multimeric binding molecule comprising an ISA as provided herein for use in treating, preventing, or managing cancer.

This disclosure employs, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Green and Sambrook, ed. (2012) Molecular Cloning A Laboratory Manual (4th ed.; Cold Spring Harbor Laboratory Press); Sambrook et al., ed. (1992) Molecular Cloning: A Laboratory Manual, (Cold Springs Harbor Laboratory, NY); D. N. Glover and B. D. Hames, eds., (1995) DNA Cloning 2d Edition (IRL Press), Volumes 1-4; Gait, ed. (1990) Oligonucleotide Synthesis (IRL Press); Mullis et al. U.S. Pat. No. 4,683,195; Hames and Higgins, eds. (1985) Nucleic Acid Hybridization (IRL Press); Hames and Higgins, eds. (1984) Transcription And Translation (IRL Press); Freshney (2016) Culture Of Animal Cells, 7th Edition (Wiley-Blackwell); Woodward, J., Immobilized Cells And Enzymes (IRL Press) (1985); Perbal (1988) A Practical Guide To Molecular Cloning; 2d Edition (Wiley-Interscience); Miller and Calos eds. (1987) Gene Transfer Vectors For Mammalian Cells, (Cold Spring Harbor Laboratory); S. C. Makrides (2003) Gene Transfer and Expression in Mammalian Cells (Elsevier Science); Methods in Enzymology, Vols. 151-155 (Academic Press, Inc., N.Y.); Mayer and Walker, eds. (1987) Immunochemical Methods in Cell and Molecular Biology (Academic Press, London); Weir and Blackwell, eds.; and in Ausubel et al. (1995) Current Protocols in Molecular Biology (John Wiley and Sons).

General principles of antibody engineering are set forth, e.g., in Strohl, W. R., and L. M. Strohl (2012), Therapeutic Antibody Engineering (Woodhead Publishing). General principles of protein engineering are set forth, e.g., in Park and Cochran, eds. (2009), Protein Engineering and Design (CDC Press). General principles of immunology are set forth, e.g., in: Abbas and Lichtman (2017) Cellular and Molecular Immunology 9th Edition (Elsevier). Additionally, standard methods in immunology known in the art can be followed, e.g., in Current Protocols in Immunology (Wiley Online Library); Wild, D. (2013), The Immunoassay Handbook 4th Edition (Elsevier Science); Greenfield, ed. (2013), Antibodies, a Laboratory Manual, 2d Edition (Cold Spring Harbor Press); and Ossipow and Fischer, eds., (2014), Monoclonal Antibodies: Methods and Protocols (Humana Press).

All of the references cited above, as well as all references cited herein, are incorporated herein by reference in their entireties.

The following examples are offered by way of illustration and not by way of limitation.

### EXAMPLES

### Example 1: Construction and Characterization of IgM-Based Immunostimulatory Agents (ISA) with a Modified J-Chain Expressing IL-15 and the IL-15Rα Sushi Domain

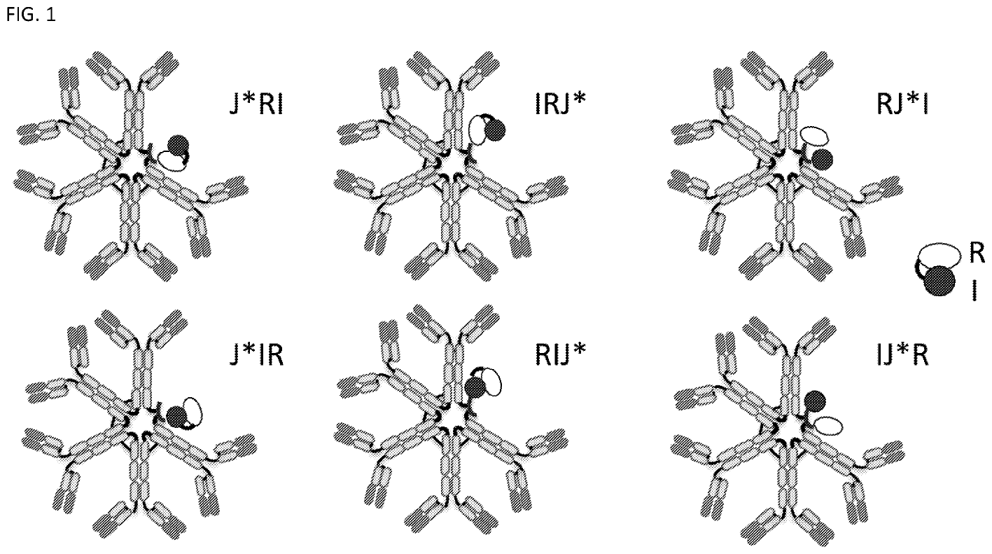

To produce IgM-based immunostimulatory agents (ISAs), modified J-chains expressing mature human IL-15 (SEQ ID NO: 4) and the sushi domain of human IL-15Rα (SEQ ID NO: 5) as a fusion protein were constructed and characterized as follows. The starting point for the modified J-chains was a variant of the mature human J-chain comprising a Y to A amino acid substitution at position 102 (“Y102A” or “J*,” amino acid sequence of the variant presented as SEQ ID NO: 3) that enhances serum half-life of IgM pentamers that comprise the J-chain variant. See PCT Publication No. WO 2019/169314A1. Initially, IgM antibodies comprising antigen binding domains that bind to PD-L1 were assembled with various fusion proteins comprising all three domains: J*, mature IL-15 (“I”) and the IL-15Rα sushi domain (“R”) in, various orientations. The modified J-chains evaluated included J*RI (SEQ ID NO: 6), J*IR (SEQ ID NO: 20), IRJ* (SEQ ID NO: 21), RIJ* (SEQ ID NO: 22), RJ*I (SEQ ID NO: 23), and IJ*R (SEQ ID NO: 24). See FIG. 1. In addition, fusion proteins including just J*I (SEQ ID NO: 26) or IJ* (SEQ ID NO: 25), as well as a human J-chain fused to human serum albumin (HSA) (SEQ ID NO: 84, “J15HSA” as disclosed in U.S. Pat. No. 10,618,978, which is incorporated herein by reference in its entirety) were constructed. DNA constructs encoding these J-chain fusion proteins were expressed along with DNA constructs encoding anti-PD-L1 IgM heavy chains and light chains comprising, respectively, the VH and VL amino acid sequences of humanized anti-human PD-L1 antibody h3C5, SEQ ID NO: 33 and SEQ ID NO: 34, respectively, as disclosed in U.S. Patent Application Publication No. 2019/0338031, which is incorporated herein by reference in its entirety. The antibodies were assessed for proper assembly as pentamers.

The IgM pentamers with the modified J-chains and corresponding IgG antibodies were constructed and expressed as described in U.S. Patent Application Publication No. 2019/0338031.

A schematic of the various orientations of the modified J-chain as IgM pentamers is presented in FIG. 1. J*RI (SEQ ID NO: 6) showed superior expression and assembly as an IgM, and thus was selected for further characterization. The construct was compared to “KD-RI,” an anti-PD-L1 IgG antibody comprising the human IL-15Rα sushi domain and human IL-15 fused to its C-terminus, as described in U.S. Pat. No. 10,407,502 (heavy chain fusion protein presented as SEQ ID NO: 29 and light chain presented as SEQ ID NO: 30).

The IgM and IgG constructs were shown to bind to PD-L1 by ELISA with binding affinities as shown in Table 2.

TABLE 2
PD-L1 Binding Affinities

| Construct | Binding Affinity K_{d}(nM) |
|---|---|
| H1-3C5 + JH | 0.05 |
| H1-3C5 + J*RI | 0.04 |
| KD-RI | 1.33 |

Alternatively, the modified J-chain J*RI (SEQ ID NO: 6) was assembled as anti-PD-L1 human IgM pentamers where the IgM heavy chain and the light chain include, respectively, the VH and VL amino acid sequences of the antibody disclosed in U.S. Pat. No. 8,217,149 presented here as SEQ ID NO: 75 and SEQ ID NO: 76, respectively. Additional IgM pentamers were also assembled with the modified J-chain J*RI, including anti-GITR IgM antibody #23 (VH and VL comprising SEQ ID NO: 39 and 40, respectively), anti-GITR IgM antibody #14 (VH and VL comprising SEQ ID NO: 41 and 42, respectively), anti-GITR IgM antibody #12 (VH and VL comprising SEQ ID NO: 43 and 44, respectively), anti CD20 IgM antibody 153 (VH and VL comprising SEQ ID NO: 49 and 50, respectively). Finally, non-J-chain constructs in which IL-15 and the IL-15 receptor α sushi domain are fused to human serum albumin (“HRI,” SEQ ID NO: 27 and “IRH,” SEQ ID NO: 28) were expressed.

### Example 2: Ki-67 In Vitro Potency Assay of IgM-Based ISAs

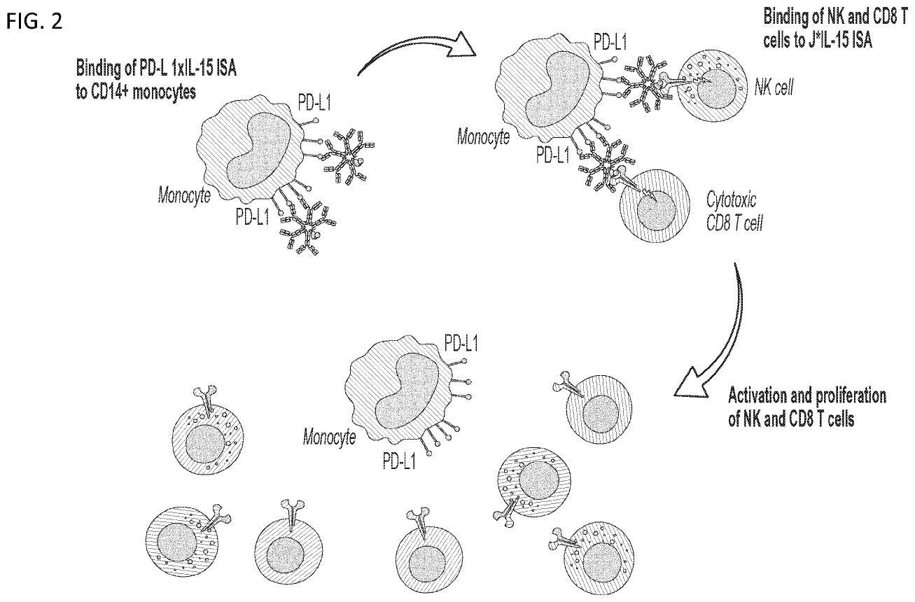

The in vitro potency of the various IgM J*RI ISA constructs prepared in Example 1 was evaluated in a Ki-67 Proliferation Assay, as follows. The assay measures proliferation of primary cells (huPBMCs, human peripheral blood mononuclear cells) in response to IL-15. Binding of IL-15 to its receptor leads to cell proliferation, which can be visualized by several techniques, one of them being a cell cycle-associated protein assay. The most common used cell cycle-associated protein is Ki-67, expressed only in G1, S, G2 and M phases. Determination of Ki-67 protein level in the nucleus of cytotoxic CD8 T cells and natural killer NK cells (cells expressing physiologically the β and γ subunits of the IL-15 receptor) by flow cytometry is a surrogate assay for actual cell proliferation. A schematic of the assay is shown in FIG. 2.

Briefly, healthy donor PBMCs were incubated in presence of a dose titration of the compounds to be tested for 3-5 days, then surface stained for T and NK cell markers, and intracellularly for Ki-67. Stained cells are acquired on a flow cytometer, and the flow data analysis focuses on the Ki-67 content of the CD8 T cells and NK cells. EC50 determination is achieved by graphing the percentage of Ki-67 positive CD8 T cells and NK cells against the compound concentration.

Protocol

huPBMCs were thawed, counted, and resuspended in RPMI-1640 medium containing 10% fetal bovine serum (FBS) at 1×10⁶ cells/mL, and 180 μL of cell suspension was added per well in U-bottom microtiter plates (cat. no. 351177, Falcon). Dose titration (1:3 dilution series) of ISAs produced as described in Example 1 or controls were done in RPMI-1640 containing 10% FBS, and 20 μL of the appropriate dilutions added to the wells containing the huPBMCs. Cells were incubated from 3-5 days at 37° C. in 5% CO₂.

The cell/ISA mixtures were then transferred in V-bottom plate (cat. no. 82.1583.001, Sarstedt) and surface-stained for 30 min at room temperature in FACS staining buffer (BD Biosciences cat. no. 554656) with the following antibodies: anti-CD3 PerCP-Cy5.5 (Biolegend cat. no. 300430), anti-CD4-Brilliant Violet-421 (Biolegend cat. no. 300532), anti-CD8a APC-Fire 750 (Biolegend cat. no. 344746), and anti-NKp46 PE-Cy7 (Biolegend cat. no. 331916). The cells were washed twice, fixed, and intracellularly stained for Ki67 (Anti-Ki-67 APC, Biolegend cat. no. 350514) and FoxP3 (anti-FoxP3 PE, Biolegend cat. no. 320108) using the Foxp3/Transcription Factor Staining Buffer Set (cat. no. 00-5523-00, eBiosciences/ThermoFisher Scientific). Cells were finally washed twice and acquired on FACSCalibur-DxP8 (BD/Cytek Biosciences).

FACS data was analyzed in FlowJo software (FlowJo LLC.) as followed: CD4 T cells (CD3+/CD4+), CD8 T cells (CD3+/CD8+), NK cells (CD3−/NKp46+), and regulatory T cell (Treg, CD4+/FoxP3+) subsets were gated and the percentage of Ki67 positive cells in each population was graphed against the antibody concentration. The EC50 for the biological activity were calculated using a Nonlinear fit with variable slope (4 parameters) in GraphPad Prism (GraphPad Software Inc.).

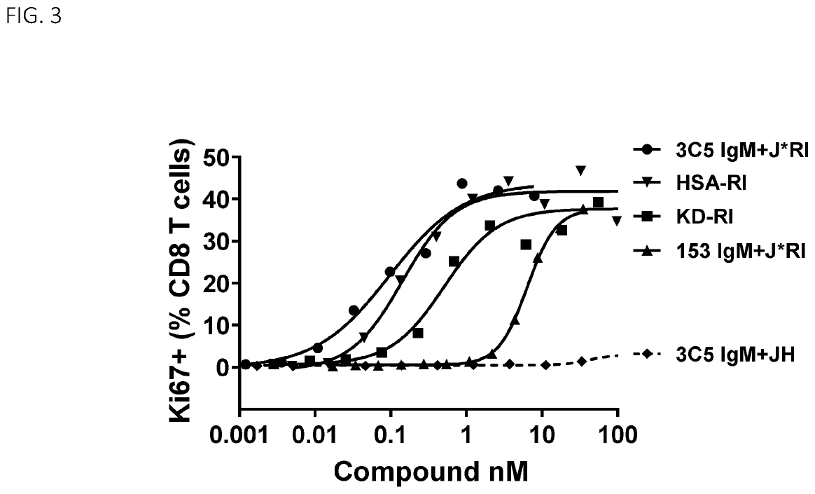

Exemplary results showing CD8 T cell proliferation in response to increasing concentrations of h3C5 IgM+J*RI, HRI, KD-RI, and 153 IgM J*RI are shown in FIG. 3 and in Table 3. Comparable results were obtained for NK cell proliferation (data not shown).

TABLE 3
CD8+ Proliferation in Response to IL-15 ISAs

| Antibody | EC50(nM) |
|---|---|
| h3C5 IgM + J*RI | 0.1 |
| HSA-RI | 0.14 |
| KD-RI | 0.51 |
| 153 IgM + J*RI | 0.64 |
| h3C5 IgM + JH | >100 |

In another assay, proliferation of CD8+ and CD4+ T cells as well as regulatory CD4+/FoxP3+ T cells (Treg) in response to increasing concentrations of h3C5 IgM+J*RI was compared. As shown in FIG. 4, 3C5 IgM+J*RI induced proliferation of CD8+ T cells but not CD4+ T cells or Treg cells.

### Example 3: Evaluation of ISAs Comprising IL-15 Variants with Reduced Receptor Binding

In certain aspects, for example to control potential toxic side of therapeutic ISAs, it is desirable to modify the potency of IL-15 ISAs by reducing binding to the IL-15 beta/gamma receptor. Nine residues in mature human IL-15 (SEQ ID NO: 4) were previously identified by others as having the capability to reduce receptor binding, see PCT Publication No. WO 2018/071918A1. These include IL-15 N1D (SEQ ID NO: 57), N4D (SEQ ID NO: 58), D8N (SEQ ID NO: 59), D30N (SEQ ID NO: 60), D61N (SEQ ID NO: 61), E64Q (SEQ ID NO: 62), N65D (SEQ ID NO: 63), N72D (SEQ ID NO: 64), and Q108E (SEQ ID NO: 65). These mutant IL-15 sequences, along with double mutants N4D/N65D (SEQ ID NO: 66) and N1D/N65D (SEQ ID NO: 67), and triple mutant D30N/E64Q/N65D (SEQ ID NO: 68) were incorporated into modified J-chains with the J*RI configuration, resulting in fusions proteins with the sequences SEQ ID Nos: 7-18, respectively. A modified J-chain comprising an IL-15 sequence with all nine mutations (SEQ ID NO: 77) was also constructed.

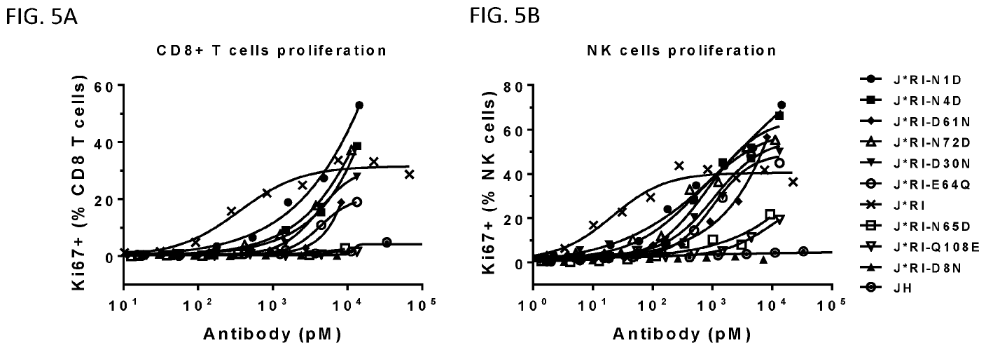

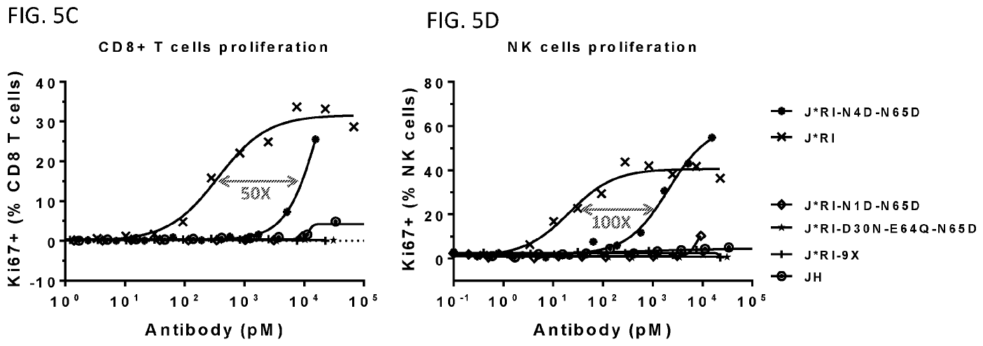

The ability of the various ISA constructs with single IL-15 mutations to trigger CD8+ T cell or NK cell proliferation is shown in FIG. 5A and FIG. 5B, respectively, and the ability of ISA constructs with the double or triple IL-15 mutations to trigger CD8+ T cell or NK cell proliferation is shown in FIG. 5C and FIG. 5D, respectively. ISAs with the various single IL-15 mutations showed a range of reduction in receptor activation. The construct with all nine mutations had no activity (data not shown). The J*RI N4D/N65D double mutant showed 50× and 100× reduced potency over the constructs with WT IL-15 on CD8+ T cell and NK cell proliferation, respectively, while the N1D/N65D double mutant and the triple mutant did not trigger proliferation of either cell type.

### Example 4: h3C5 IgM+J*RI Upregulates GITR and OX-40 on Cytotoxic CD8 T Cells

A Ki-67 proliferation assay was carried out using h3C5 IgM+J*RI as the ISA, according to the methods described in Example 2, except huPBMCs were incubated with the 5 nM of each indicated ISA for 5-6 days, and GITR, OX-40 and Ki-67 expression on CD8-gated T-cells was resolved by flow cytometry. As shown in FIG. 6, the IgM-based ISA targeting PD-L1 upregulated GITR and OX40 expression on CD8+ T cells to a greater extent than HRI, 153 IgM J*RI, KD-RI, or h3C5 IgM_JH (no IL-15).

### Example 5: Anti-GITR IgM+J*RI ISAs Show In Vitro Potency in Ki-67 CD8+ T Cell Proliferation Assay

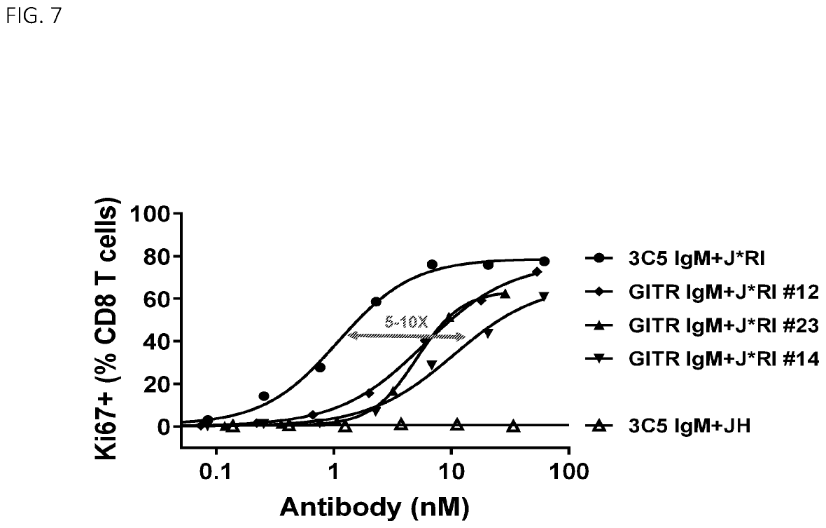

The three anti-GITR IgM+J*RI ISA compounds prepared as described in Example 1 were tested in the Ki-67 assay for their ability to trigger CD8+ T cell proliferation. The VH and VL sequences of GITR IgM_J*RI mab #23 are presented as SEQ ID NO: 39 and SEQ ID NO: 40, respectively. GITR IgM_J*RI mab #14 are presented as SEQ ID NO: 41 and SEQ ID NO: 42, respectively. GITR IgM_J*RI mab #12 are presented as SEQ ID NO: 43 and SEQ ID NO: 44, respectively. The mab numbers correspond to the GITR binders disclosed in PCT Application No. PCT/US2020/017083, which is incorporated herein by reference in its entirety. The Ki-67 assay was performed on human PBMCs as described in Example 2. The results, compared to h3C5 IgM+J*RI, are shown in FIG. 7. The three anti-GITR constructs each showed potency to trigger CD8+ T cell proliferation at a level of about 5-10 times less than the anti-PD-L1 construct.

### Example 6: h3C5 IgM+SJ*RI has Increased Potency in the Ki-67 CD8+ T Cell Proliferation Assay

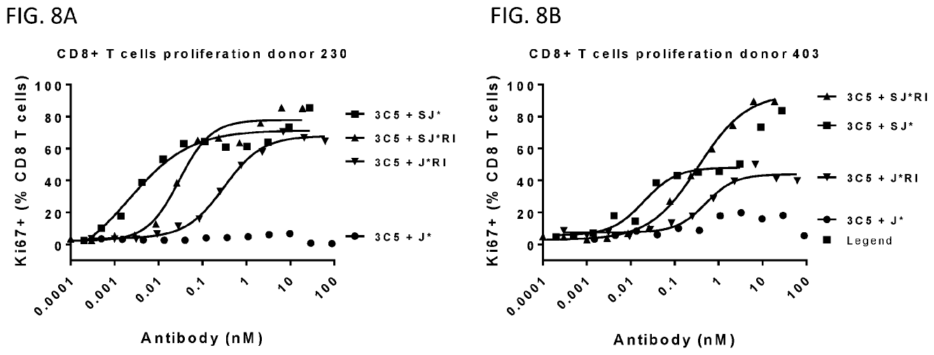

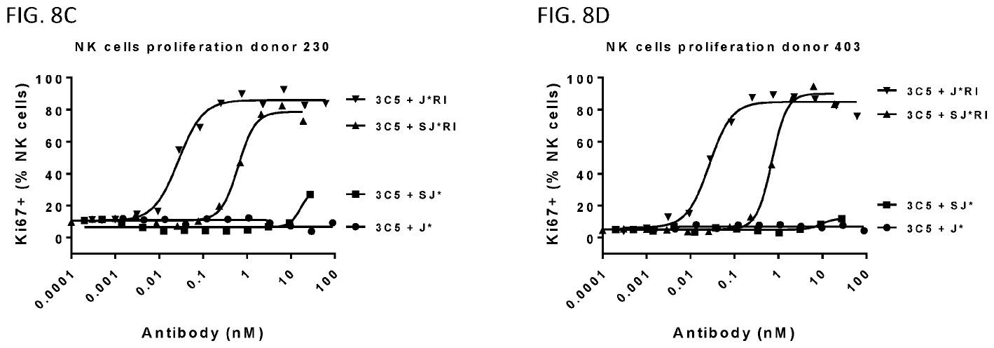

We next investigated whether fusing a T-cell-targeting antigen binding domain onto the J*RI element of the IgM-based ISAs could increase potency. A modified J-chain comprising, in N-terminal to C-terminal direction an scFv comprising the VH and VL of mouse-anti-human CD3 monoclonal antibody SP34, J*, R, and I, the latter three as described in Example 1, was constructed (SEQ ID NO: 19), and was shown to assemble properly with IgM h3C5 heavy chains and light chains to form a pentamer. The pentamer was tested in the Ki-67 proliferation assay (60-hour incubation) gated on CD8+ T cells (FIG. 8A and FIG. 8B showing two different PBMC donors) or CD3-negative NK cells (FIG. 8C and FIG. 8D showing two different PBMC donors). The construct exhibited intermediate potency for CD8+ T cell proliferation and intermediate potency for NK cell proliferation relative to h3C5 IgM+SJ*, h3C5 IgM+J*RI, or h3C5 IgM J*.

The EC50s, expressed in nM for CD8+ T cell proliferation for the two different donors is shown in Table 4, and the EC50s, expressed in NM for the NK cell proliferation for the two different donors is shown in Table 5.

TABLE 4
CD8+ T Cell Proliferation EC50s

| | h3C5 + SJ* | h3C5 + SJ*RI | h3C5 + J*RI | h3C5 + J* |
|---|---|---|---|---|
| EC₅₀ nM | 0.002 | 0.03 | 0.31 | >80 |
| Donor 230 | | | | |
| EC₅₀ nM | 0.02 | 0.34 | 0.51 | >80 |
| Donor 403 | | | | |

TABLE 5
NK Cell Proliferation EC50s

| | h3C5 + SJ* | h3C5 + SJ*RI | h3C5 + J*RI | h3C5 + J* |
|---|---|---|---|---|
| EC₅₀ nM | >80 | 0.63 | 0.027 | >80 |
| Donor 230 | | | | |
| EC₅₀ nM | >80 | 0.71 | 0.027 | >80 |
| Donor 403 | | | | |

### Example 7: Potency of Anti-PD-L1 J*RI in In Vivo Mouse Tumor Efficacy Models

The modified J-chain J*RI (SEQ ID NO: 6) was assembled as anti-PD-L1 human IgM pentamers where the IgM heavy chain and the light chain include, respectively, the VH and VL amino acid sequences SEQ ID NO: 224 and SEQ ID NO: 225, respectively, hereafter “m3c5-J*RI.” An anti-PD-L1 IgG antibody comprising the VH and VL domains of SEQ ID NO: 75 and SEQ ID NO: 76, respectively, was generated using standard techniques.

The anti-tumor effect of m3c5-J*RI was evaluated in a genetically engineered mouse model. The mouse colon carcinoma cell line CT-26 expressing human PD-L1 in lieu of mPD-L1 was implanted on the left flank in a group of BALB/c mice expressing the human PD-1 and human CTLA-4 molecules in lieu of the mouse PD-1 and CTLA-4 molecules. When the tumors reached between 60-100 mm³, mice were randomized in groups of 10 and were treated as shown in Table 6. Tumor size was measured 3 times a week for a total duration of 38 days from start of treatments.

TABLE 6
Treatment groups

| Groups n = 10 | Treatment | Dosing conc. | Dosing schedule |
|---|---|---|---|
| 1 | Vehicle | NA | ip BIW × 3⁺ |
| 2 | Anti-PD-L1 IgG | 5 mg/kg | ip Q3d × 6* |
| 3 | m3c5-J*RI | 5 mg/kg | ip Q2d × 3^{#} |
| 4 | m3c5-J*RI | 10 mg/kg | ip Q2d × 3 |
| 5 | m3c5-J*RI | 25 mg/kg | ip Q2d × 3 |

⁺ip BIW × 3: twice weekly intraperitoneally for a total of 3 injections
*ip Q3d × 6: every 3 days intraperitoneally for a total of 6 injections
^{#}ip Q2d × 3: every 2 days intraperitoneally for a total of 3 injections

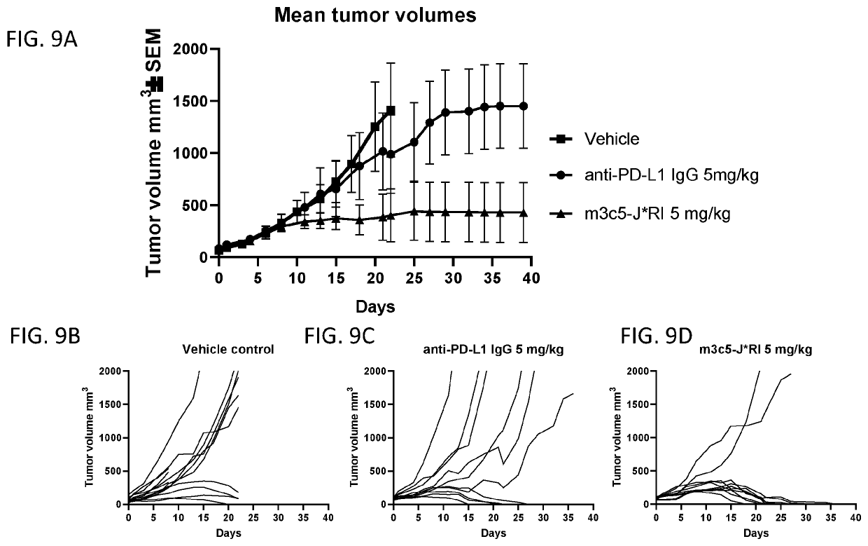

The average tumor size for each tumor group is shown in FIG. 9A. The individual tumor sizes in treatments groups 1, 2 and 3 are shown in FIGS. 9B-9D, respectively. Group 1 was terminated at day 22 (endpoint of average tumor size >1,500 mm³) and groups 2 and 3 were monitored to day 39. The number of tumor-free animals in groups 1-5 is shown in Table 7.

TABLE 7
Regressing or tumor-free mice

| | | Day 22 ≤60 | Day 39 |
|---|---|---|---|
| Groups | Treatment | mm³ | Tumor-free |
| 1 | Vehicle | 1/10 | — |
| 2 | Anti-PD-L1 IgG 5 mg/kg | 4/10 | 4/10 |
| 3 | mIGM-7354 5 mg/kg | 6/10 | 8/10 |
| 4 | mIGM-7354 10 mg/kg | 2/10 | 6/10 |
| 5 | mIGM-7354 25 mg/kg | 3/10 | 5/10 |

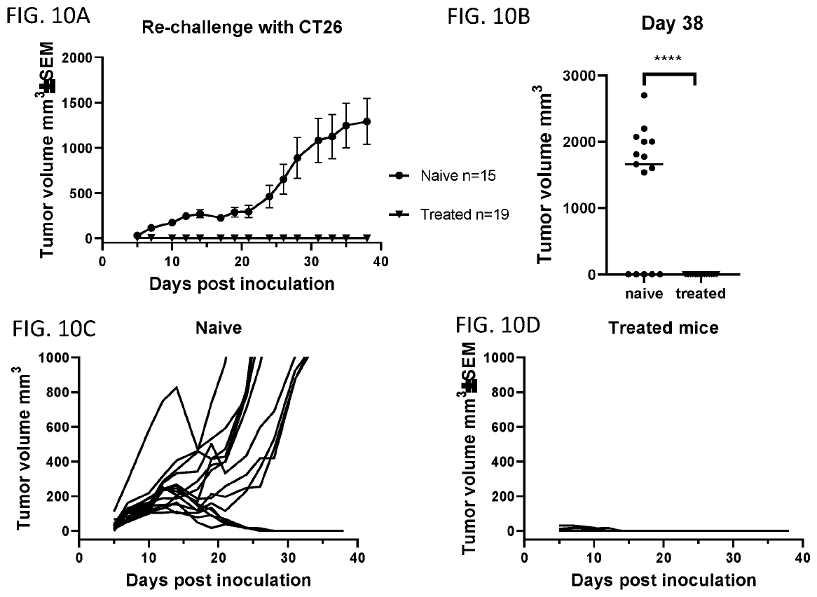

Tumor-free mice from groups 3-5 (n=19 total) as well as 15 naïve mice were re-challenged in the right flank with wild-type CT-26 tumor cells. Tumor cell growth was monitored for up to 48 days. The average tumor size for each tumor group is shown in FIG. 10A. The individual tumor sizes in naïve and treated mice are shown in FIGS. 10C and 10D, respectively. Tumors grew in 10/15 mice in the naïve group, but none grew (0/19) in the m3C5-J*RI group, showing that the anti-tumor immunity elicited by the 3 original treatments with m3C5-J*RI was long lived. FIG. 10B shows that the difference in tumor growth between the naïve and treatment groups is statistically significant. (P<0.0001).

### Example 8: Potency of Anti-PD-L1 J*RI in an In Vivo Mouse Pharmacodynamic Model

The pharmacodynamic effects of m3C5-J*RI were evaluated in a BALB/c mouse model. Groups of 5 mice were treated as shown in Table 8.

TABLE 8
Treatment groups in BALB/c pharmacodynamic model

| Groups n = 5 | Treatment | Dosing conc. | Dosing schedule |
|---|---|---|---|
| 1 | Vehicle | NA | ip Q2d × 3^{#} |
| 2 | m3C5-J*RI | 5 mg/kg | ip Q2d × 3 |
| 3 | m3C5-J*RI | 10 mg/kg | ip Q2d × 3 |
| 4 | m3C5-J*RI | 25 mg/kg | ip Q2d × 3 |

^{#}ip Q2d × 3: every 2 days intraperitoneally for a total of 3 injections

Peripheral blood NK, B cell, CD8 and CD4 T cell counts were conducted by flow cytometry as follows. Blood was collected with K2-EDTA, stained with antibodies for CD45 (leukocytes), CD3 (T cells), CD4 (CD4 T cell), CD8 (CD8 T cell), CD19 (B cell) and CD49b (NK cell) and analyzed on a flow cytometer machine with counting beads to evaluate the number of cells.

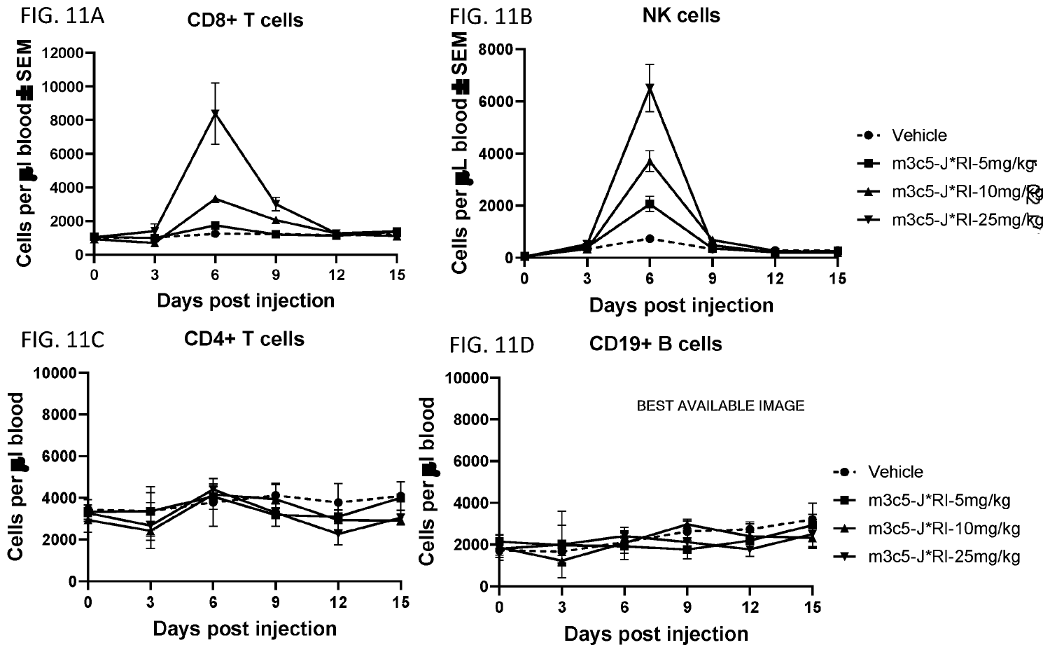

The results for CD8+ T cells, NK cells, CD4+ T cells, and CD19+ B cells are shown in FIGS. 11A-11D, respectively. There is a transient m3c5-J*RI dose-dependent increase in mouse CD8 T cells and mouse NK cells. No increase is observed with vehicle only. The proliferative effects of m3c5-J*RI do not impact CD4 T cells or B cells.

### Example 9: Evaluation of ISAs Comprising IL-15 Variants with Mutated Glycosylation Sites

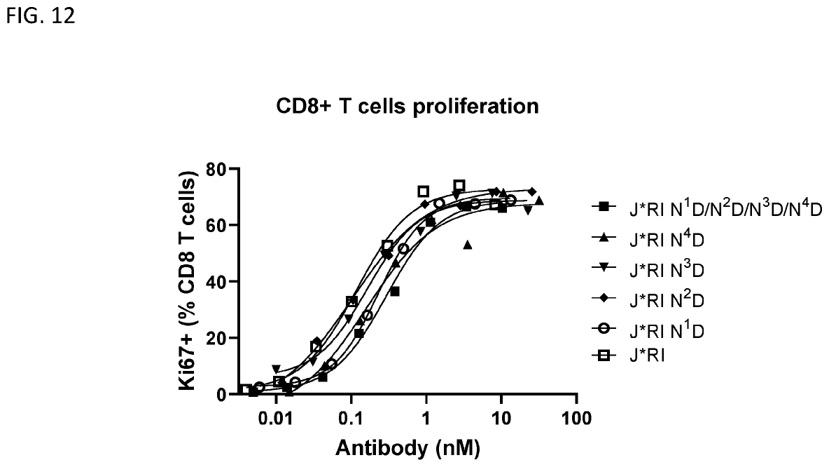

The impact of eliminating the four asparagine-based glycosylation sites on J*RI was evaluated. The first asparagine-based glycosylation site on J*RI (“N¹”) is at position 49 of J* (SEQ ID NO: 3). The other 3 asparagine-based glycosylation sites on J*RI are located in the IL-15 portion, at positions 71 “N²”, 79 “N³”, and 112 “N⁴” of SEQ ID NO: 4. J*RI sequences were generated where N¹, N², N³, N⁴ or a combination of N¹-N⁴ were mutated to an aspartic acid to remove the glycosylation sites (SEQ ID NOS: 86-90, respectively). h3C5 IgM (VH: SEQ ID NO: 33, VL: SEQ ID NO: 34)+J*RI N¹D, N²D, N³D, N⁴D, or N¹D/N²D/N³D/N⁴D mutations were generated, purified and tested for potency compared to according to the method described in Example 2. The results are shown in FIG. 12. None of the single or combination mutants showed any significant decrease in potency in comparison to the wild-type J*RI sequence in this assay

### Example 10: Evaluation of Anti-PD-L1 J*RI on CD8 T Cells from Multiple Species

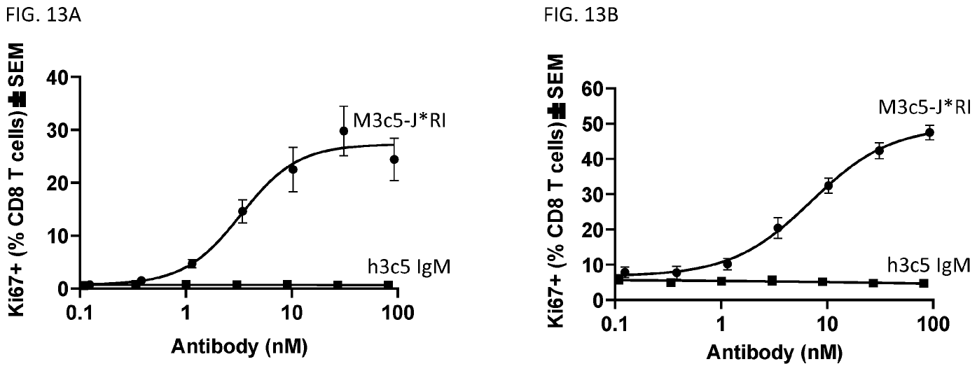

m3c5-J*RI was evaluated for its proliferative and cytokine release activity on cynomolgus PBMCs. 6 healthy human donor PBMCs as well as 4 healthy cynomolgus donor PBMCs were used side by side in the assay and incubated for 3 days with dose titrations of m3c5-J*RI and an anti-PD-L1 IgM control without IL-15 ISA, “m3c5 IgM” (VH: SEQ ID NO: 224, VL: SEQ ID NO: 225) according to the methods described in Example 2. FIGS. 13A-13B show that m3c5-J*RI has a comparable proliferative activity on human and cynomolgus CD8 T cells. Table 9 shows the average proliferative EC50 for human and cynomolgus CD8 T cells.

TABLE 9
CD8+ T cells proliferation from human and
cynomolgus in response to m3c5-J*RI and h3C5 IgM

| Antibody | Human EC50(nM) | Cynomolgus EC50(nM) |
|---|---|---|
| m3c5-J*RI | 3.22 | 6.85 |
| m3C5 IgM | >75 | >75 |

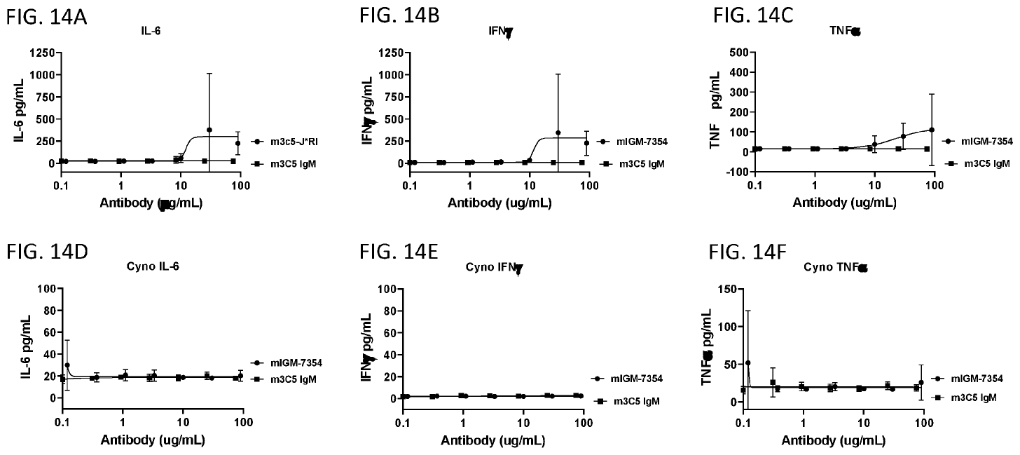

The supernatant from the proliferative assay was analyzed to determine cytokine concentration using Cytometric Bead Array (CBA) assays. Human cytokines (IL-2, IL-4, IL-6, IL-10, IFNγ and TNFα) concentration were evaluated using the human TH1/TH2 cytokine kit II according to manufacturer's instructions. Cynomolgus cytokines (IL-2, IL-4, IL-5, IL-6, IFNγ and TNFα) were evaluated using the Non-human Primate TH1/TH2 kit according to manufacturer's instructions. The resulting concentrations for human IL-6, IFNγ and TNFα and cynomolgus IL-6, IFNγ and TNFα are shown in FIGS. 14A-14F, respectively. All other cytokines were below the limit of detection.

### Example 11: Evaluation of Anti-PD-L1 J*RI in Cell-Dependent Cytotoxicity Assay

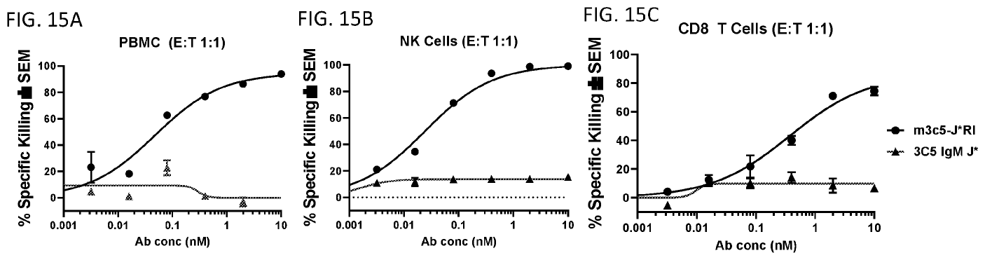

The ability of m3c5-J*RI to increase tumor cell killing using an in vitro cell-dependent cytotoxicity assay was evaluated. The human breast cancer cell line MDA-MB-231-Luc, which expresses PD-L1 and was engineered to express luciferase (Luc), was chosen as target tumor cells. PBMCs, purified NK cells, or purified CD8 T cells from healthy donors were cocultured with the MDA-MB-231-Luc at the indicated to the E:T (Effector: Target) ratios. Dose titrations of antibodies were added to the cocultures. Cells were incubated for 3 or 6 days and the luminescence resulting from the killing of MDA-MB-231-Luc was read on a EnVision Luminescence plate reader (Perkin-Elmer In.). The results for PMBCs, NK cells, and CD8 T cells are shown FIGS. 15A-15C, respectively. m3c5-J*RI increases the in vitro tumor cell killing potential of PBMCs, NK cells, and CD8 T cells.

### Example 12: Evaluation of the Epitope that 3C5 Binds on Human PD-L1

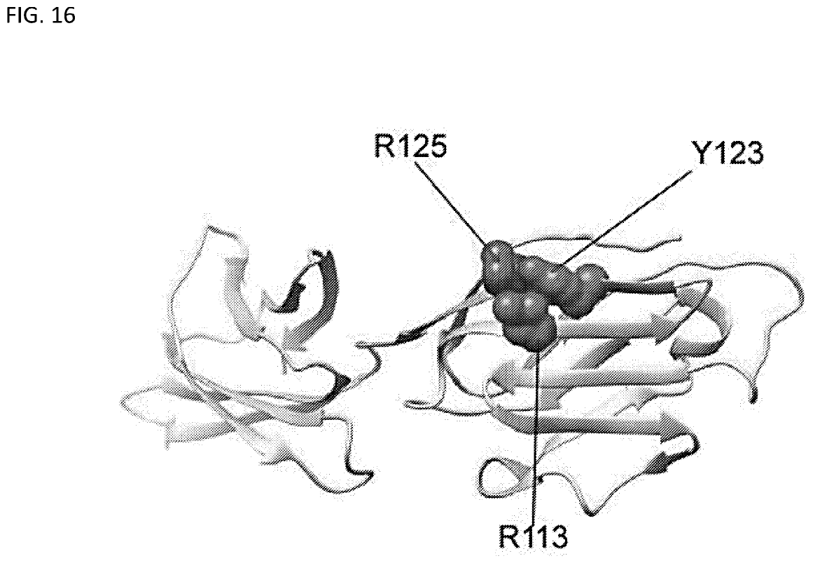

The epitope that the 3C5 H2L2 (VH: SEQ ID NO: 91, VL: SEQ ID NO: 94) antibody binds to on human PD-L1 was mapped using Alanine-scanning mutagenesis. Epitope mapping was performed by constructing an Alanine scan library of human PD-L1 which was then expressed on HEK-293T cells. Binding of the 3C5 H2L2 F(ab′)2 to each HEK-293 transfected PD-L1 mutant version was evaluated by high-throughput flow cytometry. The PD-L1 amino acids that were found to be important for 3C5 binding were R113, Y123 and R125 and the position of these amino acids on the crystal structure of PD-L1, as determined by Zhang et al. (Oncotarget, 2017, 8(52): 90215-90224) are shown in FIG. 16.

TABLE 10
Sequences Presented in the Disclosure
(signal peptides are underlined, otherwise protein is mature)

| SEQ ID | Short Name | Sequence |
|---|---|---|
| 1 | Precursor Human J | MKNHLLFWGVLAVFIKAVHVKAQEDERIVLVDNKCKCARITSRIIRSS |
| | Chain | EDPNEDIVERNIRIIVPLNNRENISDPTSPLRTRFVYHLSDLCKKCDPTEV |
| | | ELDNQIVTATQSNICDEDSATETCYTYDRNKCYTAVVPLVYGGETKM |
| | | VETALTPDACYPD |
| 2 | Mature Human J | QEDERIVLVDNKCKCARITSRIIRSSEDPNEDIVERNIRIIVPLNNRENISD |
| | Chain | PTSPLRTRFVYHLSDLCKKCDPTEVELDNQIVTATQSNICDEDSATETC |
| | | YTYDRNKCYTAVVPLVYGGETKMVETALTPDACYPD |
| 3 | Y102A mutation | QEDERIVLVDNKCKCARITSRIIRSSEDPNEDIVERNIRIIVPLNNRENISD |
| | | PTSPLRTRFVYHLSDLCKKCDPTEVELDNQIVTATQSNICDEDSATETC |
| | | ATYDRNKCYTAVVPLVYGGETKMVETALTPDACYPD |
| 4 | Mature human IL-15 | NWVNVISDLKKIEDLIQSMHIDATLYTESDVHPSCKVTAMKCFLLELQ |
| | (GenBank: amino | VISLESGDASIHDTVENLIILANNSLSSNGNVTESGCKECEELEEKNIKEF |
| | acids 23 to 136 of | LQSFVHIVQMFINTS |
| | CAA71044.1) | |
| 5 | IL-15R-alpha sushi | ITCPPPMSVEHADIWVKSYSLYSRERYICNSGFKRKAGTSSLTECVLNK |
| | domain (31-107 of | ATNVAHWTTPSLKCIRDPALVHQRPAPP |
| | NP_002180.1) | |
| 6 | J*RI Sequence | QEDERIVLVDNKCKCARITSRIIRSSEDPNEDIVERNIRIIVPLNNRENISD |
| | | PTSPLRTRFVYHLSDLCKKCDPTEVELDNQIVTATQSNICDEDSATETC |
| | | ATYDRNKCYTAVVPLVYGGETKMVETALTPDACYPDGGGGSGGGGS |
| | | GGGGSCPPPMSVEHADIWVKSYSLYSRERYICNSGFKRKAGTSSLTEC |
| | | VLNKATNVAHWTTPSLKCIRDPALVHQRPAPPSGGSGGGGSGGGSGG |
| | | GGSLQNWVNVISDLKKIEDLIQSMHIDATLYTESDVHPSCKVTAMKCF |
| | | LLELQVISLESGDASIHDTVENLIILANNSLSSNGNVTESGCKECEELEE |
| | | KNIKEFLQSFVHIVQMFINTS |
| 7 | J*RI-N1D | QEDERIVLVDNKCKCARITSRIIRSSEDPNEDIVERNIRIIVPLNNRENISD |
| | | PTSPLRTRFVYHLSDLCKKCDPTEVELDNQIVTATQSNICDEDSATETC |
| | | ATYDRNKCYTAVVPLVYGGETKMVETALTPDACYPDGGGGSGGGGS |
| | | GGGGSCPPPMSVEHADIWVKSYSLYSRERYICNSGFKRKAGTSSLTEC |
| | | VLNKATNVAHWTTPSLKCIRDPALVHQRPAPPSGGSGGGGSGGGSGG |
| | | GGSLQDWVNVISDLKKIEDLIQSMHIDATLYTESDVHPSCKVTAMKCF |
| | | LLELQVISLESGDASIHDTVENLIILANNSLSSNGNVTESGCKECEELEE |
| | | KNIKEFLQSFVHIVQMFINTS |
| 8 | J*RI-N4D | QEDERIVLVDNKCKCARITSRIIRSSEDPNEDIVERNIRIIVPLNNRENISD |
| | | PTSPLRTRFVYHLSDLCKKCDPTEVELDNQIVTATQSNICDEDSATETC |
| | | ATYDRNKCYTAVVPLVYGGETKMVETALTPDACYPDGGGGSGGGGS |
| | | GGGGSCPPPMSVEHADIWVKSYSLYSRERYICNSGFKRKAGTSSLTEC |
| | | VLNKATNVAHWTTPSLKCIRDPALVHQRPAPPSGGSGGGGSGGGSGG |
| | | GGSLQNWVDVISDLKKIEDLIQSMHIDATLYTESDVHPSCKVTAMKCF |
| | | LLELQVISLESGDASIHDTVENLIILANNSLSSNGNVTESGCKECEELEE |
| | | KNIKEFLQSFVHIVQMFINTS |
| 9 | J*RI-D8N | QEDERIVLVDNKCKCARITSRIIRSSEDPNEDIVERNIRIIVPLNNRENISD |
| | | PTSPLRTRFVYHLSDLCKKCDPTEVELDNQIVTATQSNICDEDSATETC |
| | | ATYDRNKCYTAVVPLVYGGETKMVETALTPDACYPDGGGGSGGGGS |
| | | GGGGSCPPPMSVEHADIWVKSYSLYSRERYICNSGFKRKAGTSSLTEC |
| | | VLNKATNVAHWTTPSLKCIRDPALVHQRPAPPSGGSGGGGSGGGSGG |
| | | GGSLQNWVNVISNLKKIEDLIQSMHIDATLYTESDVHPSCKVTAMKCF |
| | | LLELQVISLESGDASIHDTVENLIILANNSLSSNGNVTESGCKECEELEE |
| | | KNIKEFLQSFVHIVQMFINTS |
| 10 | J*RI-D30N | QEDERIVLVDNKCKCARITSRIIRSSEDPNEDIVERNIRIIVPLNNRENISD |
| | | PTSPLRTRFVYHLSDLCKKCDPTEVELDNQIVTATQSNICDEDSATETC |
| | | ATYDRNKCYTAVVPLVYGGETKMVETALTPDACYPDGGGGSGGGGS |
| | | GGGGSCPPPMSVEHADIWVKSYSLYSRERYICNSGFKRKAGTSSLTEC |
| | | VLNKATNVAHWTTPSLKCIRDPALVHQRPAPPSGGSGGGGSGGGSGG |
| | | GGSLQNWVNVISDLKKIEDLIQSMHIDATLYTESNVHPSCKVTAMKCF |
| | | LLELQVISLESGDASIHDTVENLIILANNSLSSNGNVTESGCKECEELEE |
| | | KNIKEFLQSFVHIVQMFINTS |
| 11 | J*RI-D61N | QEDERIVLVDNKCKCARITSRIIRSSEDPNEDIVERNIRIIVPLNNRENISD |
| | | PTSPLRTRFVYHLSDLCKKCDPTEVELDNQIVTATQSNICDEDSATETC |
| | | ATYDRNKCYTAVVPLVYGGETKMVETALTPDACYPDGGGGSGGGGS |
| | | GGGGSCPPPMSVEHADIWVKSYSLYSRERYICNSGFKRKAGTSSLTEC |
| | | VLNKATNVAHWTTPSLKCIRDPALVHQRPAPPSGGSGGGGSGGGSGG |
| | | GGSLQNWVNVISDLKKIEDLIQSMHIDATLYTESDVHPSCKVTAMKCF |
| | | LLELQVISLESGDASIHNTVENLIILANNSLSSNGNVTESGCKECEELEE |
| | | KNIKEFLQSFVHIVQMFINTS |
| 12 | J*RI-E64Q | QEDERIVLVDNKCKCARITSRIIRSSEDPNEDIVERNIRIIVPLNNRENISD |
| | | PTSPLRTRFVYHLSDLCKKCDPTEVELDNQIVTATQSNICDEDSATETC |
| | | ATYDRNKCYTAVVPLVYGGETKMVETALTPDACYPDGGGGSGGGGS |
| | | GGGGSCPPPMSVEHADIWVKSYSLYSRERYICNSGFKRKAGTSSLTEC |
| | | VLNKATNVAHWTTPSLKCIRDPALVHQRPAPPSGGSGGGGSGGGSGG |
| | | GGSLQNWVNVISDLKKIEDLIQSMHIDATLYTESDVHPSCKVTAMKCF |
| | | LLELQVISLESGDASIHDTVQNLIILANNSLSSNGNVTESGCKECEELEE |
| | | KNIKEFLQSFVHIVQMFINTS |
| 13 | J*RI-N65D | QEDERIVLVDNKCKCARITSRIIRSSEDPNEDIVERNIRIIVPLNNRENISD |
| | | PTSPLRTRFVYHLSDLCKKCDPTEVELDNQIVTATQSNICDEDSATETC |
| | | ATYDRNKCYTAVVPLVYGGETKMVETALTPDACYPDGGGGSGGGGS |
| | | GGGGSCPPPMSVEHADIWVKSYSLYSRERYICNSGFKRKAGTSSLTEC |
| | | VLNKATNVAHWTTPSLKCIRDPALVHQRPAPPSGGSGGGGSGGGSGG |
| | | GGSLQNWVNVISDLKKIEDLIQSMHIDATLYTESDVHPSCKVTAMKCF |
| | | LEELQVISLESGDASIHDTVEDLIILANNSLSSNGNVTESGCKECEELEE |
| | | KNIKEFLQSFVHIVQMFINTS |
| 14 | J*RI-N72D | QEDERIVLVDNKCKCARITSRIIRSSEDPNEDIVERNIRIIVPLNNRENISD |
| | | PTSPLRTRFVYHLSDLCKKCDPTEVELDNQIVTATQSNICDEDSATETC |
| | | ATYDRNKCYTAVVPLVYGGETKMVETALTPDACYPDGGGGSGGGGS |
| | | GGGGSCPPPMSVEHADIWVKSYSLYSRERYICNSGFKRKAGTSSLTEC |
| | | VLNKATNVAHWTTPSLKCIRDPALVHQRPAPPSGGSGGGGSGGGSGG |
| | | GGSLQNWVNVISDLKKIEDLIQSMHIDATLYTESDVHPSCKVTAMKCF |
| | | LLELQVISLESGDASIHDTVENLIILANDSLSSNGNVTESGCKECEELEE |
| | | KNIKEFLQSFVHIVQMFINTS |
| 15 | J*RI-Q108E | QEDERIVLVDNKCKCARITSRIIRSSEDPNEDIVERNIRIIVPLNNRENISD |
| | | PTSPLRTRFVYHLSDLCKKCDPTEVELDNQIVTATQSNICDEDSATETC |
| | | ATYDRNKCYTAVVPLVYGGETKMVETALTPDACYPDGGGGSGGGGS |
| | | GGGGSCPPPMSVEHADIWVKSYSLYSRERYICNSGFKRKAGTSSLTEC |
| | | VLNKATNVAHWTTPSLKCIRDPALVHQRPAPPSGGSGGGGSGGGSGG |
| | | GGSLQNWVNVISDLKKIEDLIQSMHIDATLYTESDVHPSCKVTAMKCF |
| | | LLELQVISLESGDASIHDTVENLIILANNSLSSNGNVTESGCKECEELEE |
| | | KNIKEFLQSFVHIVEMFINTS |
| 16 | J*RI-N4D/N65D | QEDERIVLVDNKCKCARITSRIIRSSEDPNEDIVERNIRIIVPLNNRENISD |
| | | PTSPLRTRFVYHLSDLCKKCDPTEVELDNQIVTATQSNICDEDSATETC |
| | | ATYDRNKCYTAVVPLVYGGETKMVETALTPDACYPDGGGGSGGGGS |
| | | GGGGSCPPPMSVEHADIWVKSYSLYSRERYICNSGFKRKAGTSSLTEC |
| | | VLNKATNVAHWTTPSLKCIRDPALVHQRPAPPSGGSGGGGSGGGSGG |
| | | GGSLQNWVDVISDLKKIEDLIQSMHIDATLYTESDVHPSCKVTAMKCF |
| | | LLELQVISLESGDASIHDTVEDLIILANNSLSSNGNVTESGCKECEELEE |
| | | KNIKEFLQSFVHIVQMFINTS |
| 17 | J*RI-N1D/N65D | QEDERIVLVDNKCKCARITSRIIRSSEDPNEDIVERNIRIIVPLNNRENISD |
| | | PTSPLRTRFVYHLSDLCKKCDPTEVELDNQIVTATQSNICDEDSATETC |
| | | ATYDRNKCYTAVVPLVYGGETKMVETALTPDACYPDGGGGSGGGGS |
| | | GGGGSCPPPMSVEHADIWVKSYSLYSRERYICNSGFKRKAGTSSLTEC |
| | | VLNKATNVAHWTTPSLKCIRDPALVHQRPAPPSGGSGGGGSGGGSGG |
| | | GGSLQDWVNVISDLKKIEDLIQSMHIDATLYTESDVHPSCKVTAMKCF |
| | | LLELQVISLESGDASIHDTVEDLIILANNSLSSNGNVTESGCKECEELEE |
| | | KNIKEFLQSFVHIVQMFINTS |
| 18 | J*RI- | QEDERIVLVDNKCKCARITSRIIRSSEDPNEDIVERNIRIIVPLNNRENISD |
| | D30N/E64Q/N65D | PTSPLRTRFVYHLSDLCKKCDPTEVELDNQIVTATQSNICDEDSATETC |
| | | ATYDRNKCYTAVVPLVYGGETKMVETALTPDACYPDGGGGSGGGGS |
| | | GGGGSCPPPMSVEHADIWVKSYSLYSRERYICNSGFKRKAGTSSLTEC |
| | | VLNKATNVAHWTTPSLKCIRDPALVHQRPAPPSGGSGGGGSGGGSGG |
| | | GGSLQNWVNVISDLKKIEDLIQSMHIDATLYTESNVHPSCKVTAMKCF |
| | | LLELQVISLESGDASIHDTVQDLIILANNSLSSNGNVTESGCKECEELEE |
| | | KNIKEFLQSFVHIVQMFINTS |
| 19 | SJ*RI | EVQLVESGGGLVQPKGSLKLSCAASGFTFNTYAMNWVRQAPGKGLE |
| | | WVARIRSKYNNYATYYADSVKDRFTISRDDSQSILYLQMNNLKTEDT |
| | | AMYYCVRHGNFGNSYVSWFAYWGQGTLVTVSSGGGGSGGGGSGGG |
| | | GSQAVVTQESALTTSPGETVTLTCRSSTGAVTTSNYANWVQEKPDHLF |
| | | TGLIGGTNKRAPGVPARFSGSLIGDKAALTITGAQTEDEAIYFCALWYS |
| | | NLWVFGGGTKLTVLGGGGSGGGGSGGGGSQEDERIVLVDNKCKCARI |
| | | TSRIIRSSEDPNEDIVERNIRIIVPLNNRENISDPTSPLRTRFVYHLSDLCK |
| | | KCDPTEVELDNQIVTATQSNICDEDSATETCATYDRNKCYTAVVPLVY |
| | | GGETKMVETALTPDACYPDGGGGSGGGGSGGGGSCPPPMSVEHADIW |
| | | VKSYSLYSRERYICNSGFKRKAGTSSLTECVLNKATNVAHWTTPSLKC |
| | | IRDPALVHQRPAPPSGGSGGGGSGGGSGGGGSLQNWVNVISDLKKIED |
| | | LIQSMHIDATLYTESDVHPSCKVTAMKCFLLELQVISLESGDASIHDTV |
| | | ENLIILANNSLSSNGNVTESGCKECEELEEKNIKEFLQSFVHIVQMFINTS |
| 20 | J*IR | QEDERIVLVDNKCKCARITSRIIRSSEDPNEDIVERNIRIIVPLNNRENISD |
| | | PTSPLRTRFVYHLSDLCKKCDPTEVELDNQIVTATQSNICDEDSATETC |
| | | ATYDRNKCYTAVVPLVYGGETKMVETALTPDACYPDGGGGSGGGGS |
| | | GGGGS |
| | | NWVNVISDLKKIEDLIQSMHIDATLYTESDVHPSCKVTAMKCFLLELQ |
| | | VISLESGDASIHDTVENLIILANNSLSSNGNVTESGCKECEELEEKNIKEF |
| | | LQSFVHIVQMFINTSSGGGSGGGGSGGGGSGGGGSGGGSLQITCPPPMS |
| | | VEHADIWVKSYSLYSRERYICNSGFKRKAGTSSLTECVLNKATNVAH |
| | | WTTPSLKCIRDPALVHQRPAPP |
| 21 | IRJ* | NWVNVISDLKKIEDLIQSMHIDATLYTESDVHPSCKVTAMKCFLLELQ |
| | | VISLESGDASIHDTVENLIILANNSLSSNGNVTESGCKECEELEEKNIKEF |
| | | LQSFVHIVQMFINTSSGGGSGGGGSGGGGSGGGGSGGGSLQITCPPPMS |
| | | VEHADIWVKSYSLYSRERYICNSGFKRKAGTSSLTECVLNKATNVAH |
| | | WTTPSLKCIRDPALVHQRPAPPGGGGSGGGGSGGGGSQEDERIVLVDN |
| | | KCKCARITSRIIRSSEDPNEDIVERNIRIIVPLNNRENISDPTSPLRTRFVY |
| | | HLSDLCKKCDPTEVELDNQIVTATQSNICDEDSATETCATYDRNKCYT |
| | | AVVPLVYGGETKMVETALTPDACYPD |
| 22 | RIJ* | ITCPPPMSVEHADIWVKSYSLYSRERYICNSGFKRKAGTSSLTECVLNK |
| | | ATNVAHWTTPSLKCIRDPALVHQRPAPPSGGSGGGGSGGGSGGGGSL |
| | | QNWVNVISDLKKIEDLIQSMHIDATLYTESDVHPSCKVTAMKCFLLEL |
| | | QVISLESGDASIHDTVENLIILANNSLSSNGNVTESGCKECEELEEKNIK |
| | | EFLQSFVHIVQMFINTSGGGGSGGGGSGGGGSQEDERIVLVDNKCKCA |
| | | RITSRIIRSSEDPNEDIVERNIRIIVPLNNRENISDPTSPLRTRFVYHLSDLC |
| | | KKCDPTEVELDNQIVTATQSNICDEDSATETCATYDRNKCYTAVVPLV |
| | | YGGETKMVETALTPDACYPD |
| 23 | RJ*I | ITCPPPMSVEHADIWVKSYSLYSRERYICNSGFKRKAGTSSLTECVLNK |
| | | ATNVAHWTTPSLKCIRDPALVHQRPAPPSGGGSGGGGSGGGGSGGGG |
| | | SGGGSLQQEDERIVLVDNKCKCARITSRIIRSSEDPNEDIVERNIRIIVPL |
| | | NNRENISDPTSPLRTRFVYHLSDLCKKCDPTEVELDNQIVTATQSNICD |
| | | EDSATETCATYDRNKCYTAVVPLVYGGETKMVETALTPDACYPDSGG |
| | | SGGGGSGGGSGGGGSLQNWVNVISDLKKIEDLIQSMHIDATLYTESDV |
| | | HPSCKVTAMKCFLLELQVISLESGDASIHDTVENLIILANNSLSSNGNVT |
| | | ESGCKECEELEEKNIKEFLQSFVHIVQMFINTS |
| 24 | IJ*R | NWVNVISDLKKIEDLIQSMHIDATLYTESDVHPSCKVTAMKCFLLELQ |
| | | VISLESGDASIHDTVENLIILANNSLSSNGNVTESGCKECEELEEKNIKEF |
| | | LQSFVHIVQMFINTSGGGGSGGGGSGGGGSQEDERIVLVDNKCKCARI |
| | | TSRIIRSSEDPNEDIVERNIRIIVPLNNRENISDPTSPLRTRFVYHLSDLCK |
| | | KCDPTEVELDNQIVTATQSNICDEDSATETCATYDRNKCYTAVVPLVY |
| | | GGETKMVETALTPDACYPDGGSGGGGSGGGSGGGGSLQCPPPMSVEH |
| | | ADIWVKSYSLYSRERYICNSGFKRKAGTSSLTECVLNKATNVAHWTTP |
| | | SLKCIRDPALVHQRPAPPSG |
| 25 | IJ* | NWVNVISDLKKIEDLIQSMHIDATLYTESDVHPSCKVTAMKCFLLELQ |
| | | VISLESGDASIHDTVENLIILANNSLSSNGNVTESGCKECEELEEKNIKEF |
| | | LQSFVHIVQMFINTSGGGGSGGGGSGGGGSQEDERIVLVDNKCKCARI |
| | | TSRIIRSSEDPNEDIVERNIRIIVPLNNRENISDPTSPLRTRFVYHLSDLCK |
| | | KCDPTEVELDNQIVTATQSNICDEDSATETCATYDRNKCYTAVVPLVY |
| | | GGETKMVETALTPDACYPD |
| 26 | J*I | QEDERIVLVDNKCKCARITSRIIRSSEDPNEDIVERNIRIIVPLNNRENISD |
| | | PTSPLRTRFVYHLSDLCKKCDPTEVELDNQIVTATQSNICDEDSATETC |
| | | ATYDRNKCYTAVVPLVYGGETKMVETALTPDACYPDGGGGSGGGGS |
| | | GGGGSNWVNVISDLKKIEDLIQSMHIDATLYTESDVHPSCKVTAMKCF |
| | | LLELQVISLESGDASIHDTVENLIILANNSLSSNGNVTESGCKECEELEE |
| | | KNIKEFLQSFVHIVQMFINTS |
| 27 | HSA Fusion HRI | DAHKSEVAHRFKDLGEENFKALVLIAFAQYLQQCPFEDHVKLVNEVT |
| | | EFAKTCVADESAENCDKSLHTLFGDKLCTVATLRETYGEMADCCAKQ |
| | | EPERNECFLQHKDDNPNLPRLVRPEVDVMCTAFHDNEETFLKKYLYEI |
| | | ARRHPYFYAPELLFFAKRYKAAFTECCQAADKAACLLPKLDELRDEG |
| | | KASSAKQRLKCASLQKFGERAFKAWAVARLSQRFPKAEFAEVSKLVT |
| | | DLTKVHTECCHGDLLECADDRADLAKYICENQDSISSKLKECCEKPLL |
| | | EKSHCIAEVENDEMPADLPSLAADFVESKDVCKNYAEAKDVFLGMFL |
| | | YEYARRHPDYSVVLLLRLAKTYETTLEKCCAAADPHECYAKVFDEFK |
| | | PLVEEPQNLIKQNCELFKQLGEYKFQNALLVRYTKKVPQVSTPTLVEV |
| | | SRNLGKVGSKCCKHPEAKRMPCAEDYLSVVLNQLCVLHEKTPVSDRV |
| | | TKCCTESLVNRRPCFSALEVDETYVPKEFNAETFTFHADICTLSEKERQI |
| | | KKQTALVELVKHKPKATKEQLKAVMDDFAAFVEKCCKADDKETCFA |
| | | EEGKKLVAASQAALGLGGGGSGGGGSGGGGSCPPPMSVEHADIWVKS |
| | | YSLYSRERYICNSGFKRKAGTSSLTECVLNKATNVAHWTTPSLKCIRDP |
| | | ALVHQRPAPPSGGSGGGGSGGGSGGGGSLQNWVNVISDLKKIEDLIQ |
| | | SMHIDATLYTESDVHPSCKVTAMKCFLLELQVISLESGDASIHDTVE |
| | | NLIILANNSLSSNGNVTESGCKECEELEEKNIKEFLQSFVHIVQMFINTS |
| 28 | HSA Fusion IRH | NWVNVISDLKKIEDLIQSMHIDATLYTESDVHPSCKVTAMKCFLLELQ |
| | | VISLESGDASIHDTVENLIILANNSLSSNGNVTESGCKECEELEEKNIKEF |
| | | LQSFVHIVQMFINTSSGGGSGGGGSGGGGSGGGGSGGGSLQITCPPPMS |
| | | VEHADIWVKSYSLYSRERYICNSGFKRKAGTSSLTECVLNKATNVAH |
| | | WTTPSLKCIRDPALVHQRPAPPGGGGSGGGGSGGGGSDAHKSEVAHR |
| | | FKDLGEENFKALVLIAFAQYLQQCPFEDHVKLVNEVTEFAKTCVADES |
| | | AENCDKSLHTLFGDKLCTVATLRETYGEMADCCAKQEPERNECFLQH |
| | | KDDNPNLPRLVRPEVDVMCTAFHDNEETFLKKYLYEIARRHPYFYAPE |
| | | LLFFAKRYKAAFTECCQAADKAACLLPKLDELRDEGKASSAKQRLKC |
| | | ASLQKFGERAFKAWAVARLSQRFPKAEFAEVSKLVTQLTKVHTECCH |
| | | GDLLECADDRADLAKYICENQDSISSKLKECCEKPLLEKSHCIAEVEND |
| | | EMPADLPSLAADFVESKDVCKNYAEAKDVFLGMFLYEYARRHPDYSV |
| | | VLLLRLAKTYETTLEKCCAAADPHECYAKVFDEFKPLVEEPQNLIKQN |
| | | CELFKQLGEYKFQNALLVRYTKKVPQVSTPTLVEVSRNLGKVGSKCC |
| | | KHPEAKRMPCAEDYLSVVLNQLCVLHEKTPVSDRVTKCCTESLVNRR |
| | | PCFSALEVDETYVPKEFNAETFTFHADICTLSEKERQIKKQTALVELVK |
| | | HKPKATKEQLKAVMDDFAAFVEKCCKADDKETCFAEEGKKLVAASQ |
| | | AALGL |
| 29 | KDRI: anti-PD- | EVQLLESGGGLVQPGGSLRLSCAASGFTFSAYRMFWVRQAPGKGLEW |
| | L1_IgG_heavy_RI | VSSIYPSGGITFYADSVKGRFTISRDNSKNTLYLQMNSLRAEDTAIYYC |
| | (US20160340429A1) | ARIKLGTVTTVDYWGQGTLVTVSSASTKGPSVFPLAPSSKSTSGGTAA |
| | | LGCLVKDYFPEPVTVSWNSGALTSGVHTFPAVLQSSGLYSLSSVVTVP |
| | | SSSLGTQTYICNVNHKPSNTKVDKRVEPKSCDKTHTCPPCPAPELLGGP |
| | | SVFLFPPKPKDTLMISRTPEVTCVVVDVSHEDPEVKFNWYVDGVEVHN |
| | | AKTKPREEQYNSTYRVVSVLTVLHQDWLNGKEYKCKVSNKALPAPIE |
| | | KTISKAKGQPREPQVYTLPPSREEMTKNQVSLTCLVKGFYPSDIAVEW |
| | | ESNGQPENNYKTTPPVLDSDGSFFLYSKLTVDKSRWQQGNVFSCSVM |
| | | HEALHNHYTQKSLSLSPGSCPPPMSVEHADIWVKSYSLYSRERYICNSG |
| | | FKRKAGTSSLTECVLNKATNVAHWTTPSLKCIRDSGGSGGGGSGGGS |
| | | GGGGSNWVNVISDLKKIEDLIQSMHIDATLYTESDVHPSCKVTAMKCF |
| | | LLELQVISLESGDASIHDTVENLIILANNSLSSNGNVTESGCKECEELEE |
| | | KNIKEFLQSFVHIVQMFINTS |
| 30 | KDRI: anti-PD- | QSALTQPASVSGSPGQSITISCTGTSSDVGAYNYVSWYQQHPGKAPKL |
| | L1_light | MIYDVSNRPSGVSNRFSGSKSGNTASLTISGLQAEDEADYYCSSYTSSS |
| | (US20160340429A1) | TRVFGTGTKVTVLGQPKANPTVTLFPPSSEELQANKATLVCLISDFYPG |
| | | AVTVAWKADGSPVKAGVETTKPSKQSNNKYAASSYLSLTPEQWKSH |
| | | RSYSCQVTHEGSTVEKTVAPTEC |
| 31 | IL2v | APASSSTKKTQLQLEHLLLDLQMILNGINNYKNPKLTRMLTAKFAMPK |
| | | KATELKHLQCLEEELKPLEEVLNGAQSKNFHLRPRDLISNINVIVLELK |
| | | GSETTFMCEYADETATIVEFLNRWITFAQSIISTLT |
| 32 | J*-IL2v | QEDERIVLVDNKCKCARITSRIIRSSEDPNEDIVERNIRIIVPLNNRENISD |
| | | PTSPLRTRFVYHLSDLCKKCDPTEVELDNQIVTATQSNICDEDSATETC |
| | | ATYDRNKCYTAVVPLVYGGETKMVETALTPDACYPDGGGGSGGGGS |
| | | GGGGSAPASSSTKKTQLQLEHLLLDLQMILNGINNYKNPKLTRMLTAK |
| | | FAMPKKATELKHLQCLEEELKPLEEVLNGAQSKNFHLRPRDLISNINVI |
| | | VLELKGSETTFMCEYADETATIVEFLNRWITFAQSIISTLT |
| 33 | h3c5H1-VH | QVQLQESGPGLVKPSETLSLTCTVSGFSLTSYDISWIRQPPGKGLEWIG |
| | | VIWTGVGTNYNPSLKSRVTISVDTSKNQFSLKLSSVTAADTAVYYCAR |
| | | DPYYYGMDYWGQGTLVTVSS |
| 34 | h3c5L1-VL | DIQMTQSPSSLSASVGDRVTITCRASQDISIWLSWYQQKPGKAPKLLIY |
| | | KASNLHTGVPSRFSGSGSGTDFTLTISSLQPEDFATYYCLQSQSFPRTFG |
| | | QGTKLEIK |
| 35 | Anti-GITR #1: | QVQLVESGGGVVQPGRSLRLSCAASGFTFSSYGMHWVRQAPGKGLE |
| | US9228016B2 VH | WVAVIWYEGSNKYYADSVKGRFTISRDNSKNTLYLQMNSLRAEDTAV |
| | | YYCARGGSMVRGDYYYGMDVWGQGTTVTVSS |
| 36 | Anti-GITR #1: | AIQLTQSPSSLSASVGDRVTITCRASQGISSALAWYQQKPGKAPKLLIY |
| | US9228016B2 VL | DASSLESGVPSRFSGSGSGTDFTLTISSLQPEDFATYYCQQFNSYPYTFG |
| | | QGTKLEIK |
| 37 | Anti-GITR #2: | QVQLVESGGGVVQPGRSLRLSCAASGFTFSSYGMHWVRQAPGKGLE |
| | US20150064204 VL | WMAVIWYVGSNKYYADSVKGRFTISRDNSKNTLYLQMNSLRAEDTA |
| | | VYYCARGGELGRDYYSGMDVWGQGTTVTVSS |
| 38 | Anti-GITR #2: | DIQMTQSPSSLSASVGDRVTITCRASQGIRNDLGWYQQKPGKAPKRLI |
| | US20150064204 VH | YAASSLQSGVPSRFSGSGSGTEFTLTISSLQPEDFATYYCQQHNSYPWT |
| | | FGQGTKVEIKR |
| 39 | Anti-GITR #23 VH | EVQLLESGGGLVQPGGSLRLSCAASGFPFSTYAIHWVRQAPGKGLEW |
| | | VSAISGSGGSTYYADSVKGRFTISRDNSKNTLYLQMNSLRAEDTAVYY |
| | | CAGPDWYFDLWGRGILVTVSS |
| 40 | Anti-GITR #23 VL | DIQMTQSPSSLSASVGDRVTITCRASQAISNSLAWYQQKPGKAPKLLIY |
| | | AASTLQRGVPSRFSGSGSGTDFTLTISSLQPEDFATYYCQQYYSTPYTF |
| | | GQGTKLEIK |
| 41 | Anti-GITR #14 VH | DIQMTQSPSSLSASVGDRVTITCRASQAISNSLAWYQQKPGKAPKLLIY |
| | | AASTLQRGVPSRFSGSGSGTDFTLTISSLQPEDFATYYCQQYYSTPYTF |
| | | GQGTKLEIK |
| 42 | Anti-GITR #14 VL | DIQMTQSPSSLSASVGDRVTITCQASQDISNYLNWYQQKPGKAPKLLIF |
| | | DASSLEAGVPSRFSGSGSGTDFTLTISSLQPEDFATYYCQQANSFPPTFG |
| | | QGTEVEIK |
| 43 | Anti-GITR #12 VH | QVQLVQSGAEVKKPGASVKVSCKASGYTFTSYAINWVRQAPGQGLE |
| | | WMGILSPSGGGTSYAPKFQGRVTMTRDTSTSTVYMELSSLRSEDTAVY |
| | | YCARGPWYFDLWGRGTLVTVSS |
| 44 | Anti-GITR #12 VL | DIQMTQSPSSLSASVGDRVTITCRASQGISNSLAWYQQKPGKAPKLLIY |
| | | AASSLQSGVPSRFSGSGSGTDFTLTISSLQPEDFATYYCQQSYSTPFTFG |
| | | PGTKVDIK |
| 45 | Anti-OX40 #1 VH | QVQLQESGPGLVKPSQTLSLTCAVYGGSFSSGYWNWIRKHPGKGLEYI |
| | WO2016057667 | GYISYNGITYHNPSLKSRITINRDTSKNQYSLQLNSVTPEDTAVYYCAR |
| | | YKYDYDGGHAMDYWGQGTLVTVSS |
| 46 | Anti-OX40 #1 VL | DIQMTQSPSSLSASVGDRVTITCRASQDISNYLNWYQQKPGKAPKLLIY |
| | WO2016057667 | YTSKLHSGVPSRFSGSGSGTDYTLTISSLQPEDFATYYCQQGSALPWTF |
| | | GQGTKVEIK |
| 47 | Anti-OX40 #2 VH | EVQLVQSGAEVKKPGASVKVSCKASGYTFTDSYMSWVRQAPGQGLE |
| | US20150307617A1 | WIGDMYPDNGDSSYNQKFRERVTITRDTSTSTAYLELSSLRSEDTAVY |
| | | YCVLAPRWYFSVWGQGTLVTVSS |
| 48 | Anti-OX40 #2 VL | DIQMTQSPSSLSASVGDRVTITCRASQDISNYLNWYQQKPGKAPKLLIY |
| | US20150307617A1 | YTSRLRSGVPSRFSGSGSGTDFTLTISSLQPEDFATYYCQQGHTLPPTFG |
| | | QGTKVEIK |
| 49 | anti-CD20 VH | EVQLVQSGAEVKKPGESLKISCKGSGYSFTSYWIGWVRQMPGKGLEW |
| | | MGIIYPGDSDTRYSPSFQGQVTISADKSITTAYLQWSSLKASDTAMYYC |
| | | ARHPSYGSGSPNFDYWGQGTLVTVSS |
| 50 | anti-CD20 VL | DIVMTQTPLSSPVTLGQPASISCRSSQSLVYSDGNTYLSWLQQRPGQPP |
| | | RLLIYKISNRFSGVPDRFSGSGAGTDFTLKISRVEAEDVGVYYCVQATQ |
| | | FPLTFGGGTKVEIK |
| 51 | Human IgM Constant | GSASAPTLFPLVSCENSPSDTSSVAVGCLAQDFLPDSITFSWKYKNNSDI |
| | region IMGT allele | SSTRGFPSVLRGGKYAATSQVLLPSKDVMQGTDEHVVCKVQHPNGNK |
| | IGHM*03 | EKNVPLPVIAELPPKVSVFVPPRDGFFGNPRKSKLICQATGFSPRQIQVS |
| | | WLREGKQVGSGVTTDQVQAEAKESGPTTYKVTSTLTIKESDWLSQSM |
| | | FTCRVDHRGLTFQQNASSMCVPDQDTAIRVFAIPPSFASIFLTKSTKLTC |
| | | LVTDLTTYDSVTISWTRQNGEAVKTHTNISESHPNATFSAVGEASICED |
| | | DWNSGERFTCTVTHTDLPSPLKQTISRPKGVALHRPDVYLLPPAREQL |
| | | NLRESATITCLVTGFSPADVFVQWMQRGQPLSPEKYVTSAPMPEPQAP |
| | | GRYFAHSILTVSEEEWNTGETYTCVVAHEALPNRVTERTVDKSTGKPT |
| | | LYNVSLVMSDTAGTCY |
| 52 | Human IgM Constant | GSASAPTLFPLVSCENSPSDTSSVAVGCLAQDFLPDSITFSWKYKNNSDI |
| | region IMGT allele | SSTRGFPSVLRGGKYAATSQVLLPSKDVMQGTDEHVVCKVQHPNGNK |
| | IGHM*04; | EKNVPLPVIAELPPKVSVFVPPRDGFFGNPRKSKLICQATGFSPRQIQVS |
| | | WLREGKQVGSGVTTDQVQAEAKESGPTTYKVTSTLTIKESDWLGQSM |
| | | FTCRVDHRGLTFQQNASSMCVPDQDTAIRVFAIPPSFASIFLTKSTKLTC |
| | | LVTDLTTYDSVTISWTRQNGEAVKTHTNISESHPNATFSAVGEASICED |
| | | DWNSGERFTCTVTHTDLPSPLKQTISRPKGVALHRPDVYLLPPAREQL |
| | | NLRESATITCLVTGFSPADVFVQWMQRGQPLSPEKYVTSAPMPEPQAP |
| | | GRYFAHSILTVSEEEWNTGETYTCVVAHEALPNRVTERTVDKSTGKPT |
| | | LYNVSLVMSDTAGTCY |
| 53 | Human IgA1 Constant | ASPTSPKVFPLSLCSTQPDGNVVIACLVQGFFPQEPLSVTWSESGQGVT |
| | Region | ARNFPPSQDASGDLYTTSSQLTLPATQCLAGKSVTCHVKHYTNPSQDV |
| | | TVPCPVPSTPPTPSPSTPPTPSPSCCHPRLSLHRPALEDLLLGSEANLTCT |
| | | LTGLRDASGVTFTWTPSSGKSAVQGPPERDLCGCYSVSSVLPGCAEPW |
| | | NHGKTFTCTAAYPESKTPLTATLSKSGNTFRPEVHLLPPPSEELALNEL |
| | | VTLTCLARGFSPKDVLVRWLQGSQELPREKYLTWASRQEPSQGTTTFA |
| | | VTSILRVAAEDWKKGDTFSCMVGHEALPLAFTQKTIDRLAGKPTHVN |
| | | VSVVMAEVDGTCY |
| 54 | Human IgA2 Constant | ASPTSPKVFPLSLDSTPQDGNVVVACLVQGFFPQEPLSVTWSESGQNV |
| | Region | TARNFPPSQDASGDLYTTSSQLTLPATQCPDGKSVTCHVKHYTNPSQD |
| | | VTVPCPVPPPPPCCHPRLSLHRPALEDLLLGSEANLTCTLTGLRDASGA |
| | | TFTWTPSSGKSAVQGPPERDLCGCYSVSSVLPGCAQPWNHGETFTCTA |
| | | AHPELKTPLTANITKSGNTFRPEVHLLPPPSEELALNELVTLTCLARGFS |
| | | PKDVLVRWLQGSQELPREKYLTWASRQEPSQGTTTFAVTSILRVAAED |
| | | WKKGDTFSCMVGHEALPLAFTQKTIDRLAGKPTHVNVSVVMAEVDG |
| | | TCY |
| 55 | Human Secretory | MLLFVLTCLLAVFPAISTKSPIFGPEEVNSVEGNSVSITCYYPPTSVNRH |
| | Component Precursor | TRKYWCRQGARGGCITLISSEGYVSSKYAGRANLTNFPENGTFVVNIA |
| | | QLSQDDSGRYKCGLGINSRGLSFDVSLEVSQGPGLLNDTKVYTVDLGR |
| | | TVTINCPFKTENAQKRKSLYKQIGLYPVLVIDSSGYVNPNYTGRIRLDI |
| | | QGTGQLLFSVVINQLRLSDAGQYLCQAGDDSNSNKKNADLQVLKPEP |
| | | ELVYEDLRGSVTFHCALGPEVANVAKFLCRQSSGENCDVVVNTLGKR |
| | | APAFEGRILLNPQDKDGSFSVVITGLRKEDAGRYLCGAHSDGQLQEGS |
| | | PIQAWQLFVNEESTIPRSPTVVKGVAGGSVAVLCPYNRKESKSIKYWC |
| | | LWEGAQNGRCPLLVDSEGWVKAQYEGRLSLLEEPGNGTFTVILNQLT |
| | | SRDAGFYWCLTNGDTLWRTTVEIKIIEGEPNLKVPGNVTAVLGETLKV |
| | | PCHFPCKFSSYEKYWCKWNNTGCQALPSQDEGPSKAFVNCDENSRLV |
| | | SLTLNLVTRADEGWYWCGVKQGHFYGETAAVYVAVEERKAAGSRD |
| | | VSLAKADAAPDEKVLDSGFREIENKAIQDPRLFAEEKAVADTRDQADG |
| | | SRASVDSGSSEEQGGSSRALVSTLVPLGLVLAVGAVAVGVARARHRK |
| | | NVDRVSIRSYRTDISMSDFENSREFGANDNMGASSITQETSLGGKEEFV |
| | | ATTESTTETKEPKKAKRSSKEEAEMAYKDFLLQSSTVAAEAQDGPQEA |
| 56 | human secretory | KSPIFGPEEVNSVEGNSVSITCYYPPTSVNRHTRKYWCRQGARGGCITL |
| | component mature | ISSEGYVSSKYAGRANLTNFPENGTFVVNIAQLSQDDSGRYKCGLGINS |
| | | RGLSFDVSLEVSQGPGLLNDTKVYTVDLGRTVTINCPFKTENAQKRKS |
| | | LYKQIGLYPVLVIDSSGYVNPNYTGRIRLDIQGTGQLLFSVVINQLRLSD |
| | | AGQYLCQAGDDSNSNKKNADLQVLKPEPELVYEDLRGSVTFHCALGP |
| | | EVANVAKFLCRQSSGENCDVVVNTLGKRAPAFEGRILLNPQDKDGSFS |
| | | VVITGLRKEDAGRYLCGAHSDGQLQEGSPIQAWQLFVNEESTIPRSPTV |
| | | VKGVAGGSVAVLCPYNRKESKSIKYWCLWEGAQNGRCPLLVDSEGW |
| | | VKAQYEGRLSLLEEPGNGTFTVILNQLTSRDAGFYWCLTNGDTLWRT |
| | | TVEIKIIEGEPNLKVPGNVTAVLGETLKVPCHFPCKFSSYEKYWCKWN |
| | | NTGCQALPSQDEGPSKAFVNCDENSRLVSLTLNLVTRADEGWYWCGV |
| | | KQGHFYGETAAVYVAVEERKAAGSRDVSLAKADAAPDEKVLDSGFR |
| | | EIENKAIQDPR |
| 57 | Mature human IL-15 | DWVNVISDLKKIEDLIQSMHIDATLYTESDVHPSCKVTAMKCFLLELQ |
| | (N1D) | VISLESGDASIHDTVENLIILANNSLSSNGNVTESGCKECEELEEKNIKEF |
| | | LQSFVHIVQMFINTS |
| 58 | Mature human IL-15 | NWVDVISDLKKIEDLIQSMHIDATLYTESDVHPSCKVTAMKCFLLELQ |
| | (N4D) | VISLESGDASIHDTVENLIILANNSLSSNGNVTESGCKECEELEEKNIKEF |
| | | LQSFVHIVQMFINTS |
| 59 | Mature human IL-15 | NWVNVISNLKKIEDLIQSMHIDATLYTESDVHPSCKVTAMKCFLLELQ |
| | (D8N) | VISLESGDASIHDTVENLIILANNSLSSNGNVTESGCKECEELEEKNIKEF |
| | | LQSFVHIVQMFINTS |
| 60 | Mature human IL-15 | NWVNVISDLKKIEDLIQSMHIDATLYTESNVHPSCKVTAMKCFLLELQ |
| | (D30N) | VISLESGDASIHDTVENLIILANNSLSSNGNVTESGCKECEELEEKNIKEF |
| | | LQSFVHIVQMFINTS |
| 61 | Mature human IL-15 | NWVNVISDLKKIEDLIQSMHIDATLYTESDVHPSCKVTAMKCFLLELQ |
| | (D61N) | VISLESGDASIHNTVENLIILANNSLSSNGNVTESGCKECEELEEKNIKEF |
| | | LQSFVHIVQMFINTS |
| 62 | Mature human IL-15 | NWVNVISDLKKIEDLIQSMHIDATLYTESDVHPSCKVTAMKCFLLELQ |
| | (E64Q) | VISLESGDASIHDTVQNLIILANNSLSSNGNVTESGCKECEELEEKNIKE |
| | | FLQSFVHIVQMFINTS |
| 63 | Mature human IL-15 | NWVNVISDLKKIEDLIQSMHIDATLYTESDVHPSCKVTAMKCFLLELQ |
| | (N65D) | VISLESGDASIHDTVEDLIILANNSLSSNGNVTESGCKECEELEEKNIKEF |
| | | LQSFVHIVQMFINTS |
| 64 | Mature human IL-15 | NWVNVISDLKKIEDLIQSMHIDATLYTESDVHPSCKVTAMKCFLLELQ |
| | (N72D) | VISLESGDASIHDTVENLIILANDSLSSNGNVTESGCKECEELEEKNIKEF |
| | | LQSFVHIVQMFINTS |
| 65 | Mature human IL-15 | NWVNVISDLKKIEDLIQSMHIDATLYTESDVHPSCKVTAMKCFLLELQ |
| | (Q108E) | VISLESGDASIHDTVENLIILANNSLSSNGNVTESGCKECEELEEKNIKEF |
| | | LQSFVHIVEMFINTS |
| 66 | Mature human IL-15 | NWVDVISDLKKIEDLIQSMHIDATLYTESDVHPSCKVTAMKCFLLELQ |
| | (N4D/N65D) | VISLESGDASIHDTVEDLIILANNSLSSNGNVTESGCKECEELEEKNIKEF |
| | | LQSFVHIVQMFINTS |
| 67 | Mature human IL-15 | DWVNVISDLKKIEDLIQSMHIDATLYTESDVHPSCKVTAMKCFLLELQ |
| | (N1D/N65D) | VISLESGDASIHDTVEDLIILANNSLSSNGNVTESGCKECEELEEKNIKEF |
| | | LQSFVHIVQMFINTS |
| 68 | Mature human IL-15 | NWVNVISDLKKIEDLIQSMHIDATLYTESNVHPSCKVTAMKCFLLELQ |
| | (D30N/E64Q/N65D) | VISLESGDASIHDTVQNLIILANDSLSSNGNVTESGCKECEELEEKNIKE |
| | | FLQSFVHIVQMFINTS |
| 69 | Cyno J-chain (mature | MKNHLLFWGVLAIFVKAVHVKAQEGERIVLVDNKCKCARITSRIIRSS |
| | 24-159) EHH53748.1 | EDPNEDIVERHIRIIVPLNNRENISDPTSPLRTKFVYHLSDLCKKCDPTEV |
| | | ELDNQIVTATQSNICDEDSATETCYTYDRNKCYTAVVPLTYGGETKM |
| | | VQTALTPDSCYPD |
| 70 | Mouse J-chain (mature | MKTHLLLWGVLAIFVKAVLVTGDDEATILADNKCMCTRVTSRIIPSTE |
| | 22-159) | DPNEDIVERNIRIVVPLNNRENISDPTSPLRRNFVYHLSDVCKKCDPVE |
| | | VELEDQVVTATQSNICNEDDGVPETCYMYDRNKCYTTMVPLRYHGET |
| | | KMVQAALTPDSCYPD |
| 71 | Cyno IL-15 (mature- | IFQKPHLRSVSIHCYLCLLLNSHFLTEAGIHVFILGCFSAGLPKTEANWV |
| | 48-161) EHH53989.1 | NVISDLKKIEDLIQSMHIDATLYTESDVHPSCKVTAMKCFLLELQVISH |
| | | ESGDTDIHDTVENLIILANNILSSNGNITESGCKECEELEEKNIKEFLQSF |
| | | VHIVQMFINTS |
| 72 | Mouse IL-15 (mature- | MKILKPYMRNTSISCYLCFLLNSHFLTEAGIHVFILGCVSVGLPKTEAN |
| | 49-162) sp\|P48346.1 | WIDVRYDLEKIESLIQSIHIDTTLYTDSDFHPSCKVTAMNCFLLELQVIL |
| | | HEYSNMTLNETVRNVLYLANSTLSSNKNVAESGCKECEELEEKTFTEF |
| | | LQSFIRIVQMFINTS |
| 73 | Cyno interleukin-15 | DHGITCPPPVSVEHADIRVKSYSLYSRERYICNSGFKRKAGTSSLTECVL |
| | receptor alpha, partial | NKATNIAHWTTPSLKCIRDPLLARQRPAPPFTVTTAGVTPQPESLSPSG |
| | ACI42785.1Length: | KEPAASSPSSNTTAATTAAIVPSSRLMPSTSSSTGTTEIGSHESSHGPSQT |
| | 239 (aas 4-80) | TAKTWELTASASHQPPGVYPQGHSDTTVAISTSTVLLCGLSAVSLLAC |
| | | YIKSRQTAPPASIEMEAMEALPVTGETSSRDEDLENCSHDL |
| 74 | Mouse IL-15 receptor | MASPQLRGYGVQAIPVLLLLLLLLLLPLRVTPGTTCPPPVSIEHADIRVK |
| | (sushi = aas 34-98) | NYSVNSRERYVCNSGFKRKAGTSTLIECVINKNTNVAHWTTPSLKCIR |
| | sp\|Q60819.1 | DPSLAHYSPVPTVVTPKVTSQPESPSPSAKEPEAFSPKSDTAMTTETAIM |
| | subunit alpha | PGSRLTPSQTTSAGTTGTGSHKSSRAPSLAATMTLEPTASTSLRITEISPH |
| | | SSKMTKVAISTSVLLVGAGVVMAFLAWYIKSRQPSQPCRVEVETMET |
| | | VPMTVRASSKEDEDTGA |
| 75 | anti-PDL1 antibody | EVQLVESGGGLVQPGGSLRLSCAASGFTFSDSWIHWVRQAPGKGLEW |
| | YW243.55.S70 VH | VAWISPYGGSTYYADSVKGRFTISADTSKNTAYLQMNSLRAEDTAVY |
| | USP 8217149 | YCARRHWPGGFDYWGQGTLVTVSA |
| 76 | anti-PDL1 antibody | DIQMTQSPSSLSASVGDRVTITCRASQDVSTAVAWYQQKPGKAPKLLI |
| | YW243.55.S70 VL | YSASFLYSGVPSRFSGSGSGTDFTLTISSLQPEDFATYYCQQYLYHPATF |
| | USP 8217149 | GQGTKVEIKR |
| 77 | J*RI-9X | QEDERIVLVDNKCKCARITSRIIRSSEDPNEDIVERNIRIIVPLNNRENISD |
| | | PTSPLRTRFVYHLSDLCKKCDPTEVELDNQIVTATQSNICDEDSATETC |
| | | ATYDRNKCYTAVVPLVYGGETKMVETALTPDACYPDGGGGSGGGGS |
| | | GGGGSCPPPMSVEHADIWVKSYSLYSRERYICNSGFKRKAGTSSLTEC |
| | | VLNKATNVAHWTTPSLKCIRDPALVHQRPAPPSGGSGGGGSGGGSGG |
| | | GGSLQDWVDVISNLKKIEDLIQSMHIDATLYTESNVHPSCKVTAMKCF |
| | | LLELQVISLESGDASIHNTVODLIILANDSLSSNGNVTESGCKECEELEE |
| | | KNIKEFLQSFVHIVEMFINTS |
| 78 | Linker #1 | GGGGSGGGGSGGGGS |
| 79 | Linker #2 | GGSGGGGSGGGSGGGGSLQ |
| 80 | Five Linker | GGGGS |
| 81 | Ten Linker | GGGGSGGGGS |
| 82 | Twenty Linker | GGGGSGGGGSGGGGSGGGGS |
| 83 | Twenty-five Linker | GGGGSGGGGSGGGGSGGGGSGGGGS |
| 84 | J15HSA | QEDERIVLVDNKCKCARITSRIIRSSEDPNEDIVERNIRIIVPLNNRENISD |
| | | PTSPLRTRFVYHLSDLCKKCDPTEVELDNQIVTATQSNICDEDSATETC |
| | | YTYDRNKCYTAWPLVYGGETKMVETALTPDACYPDGGGGSGGGGS |
| | | GGGGSDAHI<SEVAHRFI<DLGEENFI<ALVLIAFAQYLQQCPFEDHVKL |
| | | VNEVTEFAKTCVADESAENCDKSLHTLFGDKLCTVATLRETYGEMAD |
| | | CCAKQEPERNECFLQHKDDNPNLPRLVRPEVDVMCTAFHDNEETFLK |
| | | KYLYEIARRHPYFYAPELLFFAKRYKAAFTECCQAADKAACLLPKLDE |
| | | LRDEGKASSAKQRLKCASLQKFGERAFKAWAVARLSQRFPKAEFAEV |
| | | SKLVTDLTKVHTECCHGDLLECADDRADLAKYICENQDSISSKLKECC |
| | | EKPLLEKSHCIAEVENDEMPADLPSLAADFVESKDVCKNYAEAKDVFL |
| | | GMFLYEYARRHPDYSVVLLLRLAKTYETTLEKCCAAADPHECYAKVF |
| | | DEFKPLVEEPQNLIKQNCELFKQLGEYKFQNALLVRYTKKVPQVSTPT |
| | | LVEVSRNLGKVGSKCCKHPEAKRMPCAEDYLSVVLNQLCVLHEKTPV |
| | | SDRVTKCCTESLVNRRPCFSALEVDETYVPKEFNAETFTFHADICTLSE |
| | | KERQIKKQTALVELVKHKPKATKEQLKAVMDDFAAFVEKCCKADEDK |
| | | ETCFAEEGPKLVAASQAALGL |
| 85 | J*RI-S73I | QEDERIVLVDNKCKCARITSRIIRSSEDPNEDIVERNIRIIVPLNNRENISD |
| | | PTSPLRTRFVYHLSDLCKKCDPTEVELDNQIVTATQSNICDEDSATETC |
| | | ATYDRNKCYTAVVPLVYGGETKMVETALTPDACYPDGGGGSGGGGS |
| | | GGGGSCPPPMSVEHADIWVKSYSLYSRERYICNSGFKRKAGTSSLTEC |
| | | VLNKATNVAHWTTPSLKCIRDPALVHQRPAPPSGGSGGGGSGGGSGG |
| | | GGSLQNWVNVISDLKKIEDLIQSMHIDATLYTESDVHPSCKVTAMKCF |
| | | LLELQVISLESGDASIHDTVENLIILANNILSSNGNVTESGCKECEELEEK |
| | | NIKEFLQSFVHIVQMFINTS |
| 86 | >J*RI-N¹D | QEDERIVLVDNKCKCARITSRIIRSSEDPNEDIVERNIRIIVPLNNREDISD |
| | | PTSPLRTRFVYHLSDLCKKCDPTEVELDNQIVTATQSNICDEDSATETC |
| | | ATYDRNKCYTAVVPLVYGGETKMVETALTPDACYPDGGGGSGGGGS |
| | | GGGGSCPPPMSVEHADIWVKSYSLYSRERYICNSGFKRKAGTSSLTEC |
| | | VLNKATNVAHWTTPSLKCIRDPALVHQRPAPPSGGSGGGGSGGGSGG |
| | | GGSLQNWVNVISDLKKIEDLIQSMHIDATLYTESDVHPSCKVTAMKCF |
| | | LLELQVISLESGDASIHDTVENLIILANNSLSSNGNVTESGCKECEELEE |
| | | KNIKEFLQSFVHIVQMFINTS |
| 87 | >J*RI-N²D | QEDERIVLVDNKCKCARITSRIIRSSEDPNEDIVERNIRIIVPLNNRENISD |
| | | PTSPLRTRFVYHLSDLCKKCDPTEVELDNQIVTATQSNICDEDSATETC |
| | | ATYDRNKCYTAVVPLVYGGETKMVETALTPDACYPDGGGGSGGGGS |
| | | GGGGSCPPPMSVEHADIWVKSYSLYSRERYICNSGFKRKAGTSSLTEC |
| | | VLNKATNVAHWTTPSLKCIRDPALVHQRPAPPSGGSGGGGSGGGSGG |
| | | GGSLQNWVNVISDLKKIEDLIQSMHIDATLYTESDVHPSCKVTAMKCF |
| | | LLELQVISLESGDASIHDTVENLIILADNSLSSNGNVTESGCKECEELEE |
| | | KNIKEFLQSFVHIVQMFINTS |
| 88 | >J*RI-N³D | QEDERIVLVDNKCKCARITSRIIRSSEDPNEDIVERNIRIIVPLNNRENISD |
| | | PTSPLRTRFVYHLSDLCKKCDPTEVELDNQIVTATQSNICDEDSATETC |
| | | ATYDRNKCYTAVVPLVYGGETKMVETALTPDACYPDGGGGSGGGGS |
| | | GGGGSCPPPMSVEHADIWVKSYSLYSRERYICNSGFKRKAGTSSLTEC |
| | | VLNKATNVAHWTTPSLKCIRDPALVHQRPAPPSGGSGGGGSGGGSGG |
| | | GGSLQNWVNVISDLKKIEDLIQSMHIDATLYTESDVHPSCKVTAMKCF |
| | | LLELQVISLESGDASIHDTVENLIILANNSLSSNGDVTESGCKECEELEE |
| | | KNIKEFLQSFVHIVQMFINTS |
| 89 | >J*RI-N⁴D | QEDERIVLVDNKCKCARITSRIIRSSEDPNEDIVERNIRIIVPLNNRENISD |
| | | PTSPLRTRFVYHLSDLCKKCDPTEVELDNQIVTATQSNICDEDSATETC |
| | | ATYDRNKCYTAVVPLVYGGETKMVETALTPDACYPDGGGGSGGGGS |
| | | GGGGSCPPPMSVEHADIWVKSYSLYSRERYICNSGFKRKAGTSSLTEC |
| | | VLNKATNVAHWTTPSLKCIRDPALVHQRPAPPSGGSGGGGSGGGSGG |
| | | GGSLQNWVNVISDLKKIEDLIQSMHIDATLYTESDVHPSCKVTAMKCF |
| | | LLELQVISLESGDASIHDTVENLIILANNSLSSNGNVTESGCKECEELEE |
| | | KNIKEFLQSFVHIVQMFIDTS |
| 90 | >J*RI- | QEDERIVLVDNKCKCARITSRIIRSSEDPNEDIVERNIRIIVPLNNREDISD |
| | N¹D/N²D/N³D/N⁴D | PTSPLRTRFVYHLSDLCKKCDPTEVELDNQIVTATQSNICDEDSATETC |
| | | ATYDRNKCYTAVVPLVYGGETKMVETALTPDACYPDGGGGSGGGGS |
| | | GGGGSCPPPMSVEHADIWVKSYSLYSRERYICNSGFKRKAGTSSLTEC |
| | | VLNKATNVAHWTTPSLKCIRDPALVHQRPAPPSGGSGGGGSGGGSGG |
| | | GGSLQNWVNVISDLKKIEDLIQSMHIDATLYTESDVHPSCKVTAMKCF |
| | | LLELQVISLESGDASIHDTVENLIILADNSLSSNGDVTESGCKECEELEE |
| | | KNIKEFLQSFVHIVQMFIDTS |
| 91 | h3c5H2-VH | QVQLQESGPGLVKPSETLSLTCTVSGFSLTSYDISWIRQPPGKGLEWLG |
| | | VIWTGVGTNYNPSLKSRVTISKDTSKNQFSLKLSSVTAADTAVYYCAR |
| | | DPYYYGMDYWGQGTLVTVSS |
| 92 | h3c5H3-VH | QVQLQESGPGLVKPSETLSITCTVSGFSLTSYDISWVRQPPGKGLEWLG |
| | | VIWTGVGTNYNPSFKSRLTISKDTSKNQVSLKMSSLTAADTAVYYCVR |
| | | DPYYYGMDYWGQGTLVTVSS |
| 93 | h3c5H4-VH | QVQLQESGPGLVKPSETLSITCTVSGFSLTSYDISWIRQPPGKGLEWLG |
| | | VIWTGVGTNYNPSFKSRLTISKDNSKNQVSLKMSSLTAADTAVYYCVR |
| | | DPYYYGMDYWGQGTLVTVSS |
| 94 | h3c5L2-VL | DIQMTQSPSSLSASVGDRITITCRASQDISIWLSWYQQKPGKAPKLLIYK |
| | | ASNLHTGVPSRFSGSGSGTDFTLTISSLQPEDFATYYCLQSQSFPRTFGQ |
| | | GTKLEIK |
| 95 | h3c5H2 IgM K315D | QVQLQESGPGLVKPSETLSLTCTVSGFSLTSYDISWIRQPPGKGLEWLG |
| | | VIWTGVGTNYNPSLKSRVTISKDTSKNQFSLKLSSVTAADTAVYYCAR |
| | | DPYYYGMDYWGQGTLVTVSSGSASAPTLFPLVSCENSPSDTSSVAVGC |
| | | LAQDFLPDSITFSWKYKNNSDISSTRGFPSVLRGGKYAATSQVLLPSKD |
| | | VMQGTDEHVVCKVQHPNGNKEKNVPLPVIAELPPKVSVFVPPRDGFF |
| | | GNPRKSKLICQATGFSPRQIQVSWLREGKQVGSGVTTDQVQAEAKESG |
| | | PTTYKVTSTLTIKESDWLSQSMFTCRVDHRGLTFQQNASSMCVPDQDT |
| | | AIRVFAIPPSFASIFLTKSTKLTCLVTDLTTYDSVTISWTRQNGEAVKTH |
| | | TNISESHPNATFSAVGEASICEDDWNSGERFTCTVTHTDLPSPLDQTISR |
| | | PKGVALHRPDVYLLPPAREQLNLRESATITCLVTGFSPADVFVQWMQR |
| | | GQPLSPEKYVTSAPMPEPQAPGRYFAHSILTVSEEEWNTGETYTCVVA |
| | | HEALPNRVTERTVDKSTGKPTLYNVSLVMSDTAGTCY |
| 224 | m3c5-VH | QVQLKESGPGLVAPSQSLSITCTVSGFSLTSYDISWVRQPPGKGLEWLG |
| | | VIWTGVGTNYNSAFMSRLSISKDNSKSQVFLKMNSLQTDDTAMYYCV |
| | | RDPYYYGMDYWGQGTSVTVSS |
| 225 | m3c5-VL | DIQMNQSPSSLSASLGDTITITCRASQDISIWLSWYQQKPGNIPELLIYK |
| | | ASNLHTGVPPRFSGSGSGTDFTLTISSLQPEDIATYYCLQSQSFPRTFGG |
| | | GTKLEIK |

TABLE 11
Additional anti-PD-L1 VH and VL Sequences

| | SEQ | | SEQ | |
|---|---|---|---|---|
| Citation | ID | VH | ID | VL |
| US9988452B2 | 96 | EVQLVQSGAEVKKPGATVKISCKVSGYTFTSYWMYW | 97 | DVVMTQSPLSLPVTLGQPASISCKASQDVG |
| | | VRQATGQGLEWMGRIDPNSGSTKYNEKFKNRVTITA | | TAVAWYQQKPGQAPRLLIYWASTRHTGVPS |
| | | DKSTSTAYMELSSLRSEDTAVYYCARDYRKGLYAMD | | RFSGSGSGTEFTLTISSLQPDDFATYYCQQ |
| | | YWGQGTTVTVSS | | YNSYPLTFGQGTKVEIK |
| US9988452B2 | 98 | EVQLVQSGAEVKKPGESLRISCKGSGYTFTSYWMYW | 99 | EIVLTQSPDFQSVTPKEKVTITCKASQDVG |
| | | VRQAPGQGLEWMGRIDPNSGSTKYNEKFKNRVTISV | | TAVAWYLQKPGQSPQLLIYWASTRHTGVPS |
| | | DTSKNQFSLKLSSVTAADTAVYYCARDYRKGLYAMD | | RFSGSGSGTDFTFTISSLQPEDIATYYCQQ |
| | | YWGQGTTVTVSS | | YNSYPLTFGQGTKVEIK |
| US9988452B2 | 100 | EVQLVQSGAEVKKPGATVKISCKVSGYTFTSYWMYW | 101 | AIQLTQSPSSLSASVGDRVTITCKASQDVG |
| | | VRQARGQRLEWIGRIDPNSGSTKYNEKFKNRFTISR | | TAVAWYLQKPGQSPQLLIYWASTRHTGVPS |
| | | DNSKNTLYLQMNSLRAEDTAVYYCARDYRKGLYAMD | | RFSGSGSGTDFTFTISSLEAEDAATYYCQQ |
| | | YWGQGTTVTVSS | | YNSYPLTFGQGTKVEIK |
| US8552154 | 102 | EVQLVQSGAEVKKPGASVKVSCKASGYTFTSYVMHW | 103 | DIVLTQSPATLSLSPGERATLSCRATESVE |
| | | VRQAPGQRLEWIGYVNPFNDGTKYNEMFKGRATLTS | | YYGTSLVQWYQQKPGQPPKLLIYAASSVDS |
| | | DKSTSTAYMELSSLRSEDTAVYYCARQAWGYPWGQG | | GVPSRFSGSGSGTDFTLTINSLEAEDAAMY |
| | | TLVTVSS | | FCQQSRRVPYTFGQGTKLEIK |
| US8552154 | 104 | EVQLVQSGAEVKKPGASVKMSCKASGYTFTSYVMHW | 105 | DIVLTQSPATLSLSPGERATLSCRATESVE |
| | | VKQAPGQRLEWIGYVNPFNDGTKYNEMFKGRATLTS | | YYGTSLVQWYQQKPGQPPKLLIYAASSVDS |
| | | DKSTSTAYMELSSLRSEDTAVYYCARQAWGYPWGQG | | GVPSRFSGSGSGTDFTLTINSLEAEDAATY |
| | | TLVTVSS | | FCQQSRRVPYTFGQGTKLEIK |
| US8552154 | 106 | EVQLVQSGAEVKKPGASVKMSCKASGYTFTSYVMHW | 107 | DIVLTQSPASLALSPGERATLSCRATESVE |
| | | VKQAPGQRLEWIGYVNPFNDGTKYNEMFKGRATLTS | | YYGTSLVQWYQQKPGQPPKLLIYAASSVDS |
| | | DKSTSTAYMELSSLRSEDTAVYYCARQAWGYPWGQG | | GVPSRFSGSGSGTDFTLTINSLEEEDAAMY |
| | | TLVTVSS | | FCQQSRRVPYTFGQGTKLEIK |
| US8552154 | 108 | EVQLVQSGAEVKKPGASVKMSCKASGYTFTSYVMHW | 109 | DIVLTQSPATLSLSPGERATLSCRATESVE |
| | | VKQAPGQRLEWIGYVNPFNDGTKYNEMFKGRATLTS | | YYGTSLVQWYQQKPGQPPKLLIYAASSVDS |
| | | DKSTSTAYMELSSLRSEDTAVYYCARQAWGYPWGQG | | GVPSRFSGSGSGTDFTLTINSLEAEDAAMY |
| | | TLVTVSS | | FCQQSRRVPYTFGQGTKLEIK |
| US8552154 | 110 | EVQLQQSGPELVKPGASVKMSCKASGYTFTSYVMHW | 111 | DIVLTQSPASLAVSLGQRATISCRATESVE |
| | | VKQKPGQGLEWIGYVNPFNDGTKYNEMFKGKATLTS | | YYGTSLVQWYQQKPGQPPKLLIYAASSVDS |
| | | DKSSSTAYMELSSLTSEDSAVYYCARQAWGYPWGQG | | GVPARFSGSGSGTDFSLTIHPVEEDDIAMY |
| | | TLVTVSA | | FCQQSRRVPYTFGGGTKLEIK |
| US10058609B2 | 112 | EVQLVQSGAEVKKPGASVKVSCKASGYTFTGYYMHW | 113 | DIVMTQSPSSLSASVGDRVTITCRASQSIS |
| | | VRQAPGQGLEWMGWINPNSDNTGSAQKFQGRVFMTK | | SYLNWYQQKPGKAPKLLIYAASSLQSGVPS |
| | | TTSLNTAYMELSGLRSEDTAIYYCARERSSGYFDFW | | RFSGSGSGTDFTLTISSLQPEDFATYYCQQ |
| | | GQGTLVTVSS | | SYSTPITFGQGTRLEIK |
| US10058609B2 | 114 | QVQLVQSGAEVKKPGASVKVSCKTSGNTFTNYYMHW | 115 | DIVMTQSPPSLSASVGDRVTITCRASQSIS |
| | | VRQAPGQGLEWMGIMNPSGGSTSYAQKFQGRVTMTR | | SYLNWYQQKPGKAPKLLIYAASSLQSGVPS |
| | | DKSTSTVYMELSSLTSEDTAVYYCARDLFPHIYGNY | | RFSGSGSGTDFTLTISSLQPEDFATYYCQQ |
| | | YGMDIWGQGTTVTVSS | | SYSTPYTFGQGTKVEIK |
| US10058609B2 | 116 | QMQLVQSGAEVKKPGSSVKVSCKASGGTFSSYAISW | 117 | SYELMQPPSVSVAPGKTATIACGGENIGRK |
| | | VRQAPGQGLEWMGGIIPIFGTANYAQKFQGRVTITA | | TVHWYQQKPGQAPVLVIYYDSDRPSGIPER |
| | | DESTSTAYMELSSLRSEDTAVYYCARGNIVATITPL | | FSGSNSGNTATLTISRVEAGDEADYYCQVW |
| | | DYWGQGTLVTVSS | | DSSSDHRIFGGGTKLTVL |
| US10058609B2 | 118 | EVQLVQSGGGLVQPGGSLRLSCAASGFTFSSYSMNW | 119 | EIVLTQSPSSLSASIGDRVTLTCRASQSIR |
| | | VRQAPGKGLEWVSYISSSSSTIYYADSVKGRFTISR | | RFLNWYQQKPGKAPELLIYTASSLQSGVPS |
| | | DNAKNSLYLQMNSLRDEDTAVYYCARGDYYYGMDVW | | RFSGSGSGTDFTLTINSLQPEDFATYYCQ |
| | | GQGTTVTVSS | | QSYAVSPYTFGQGTKVEIR |
| US10058609B2 | 120 | EVQLVESGAEVKKPGSSVKVSCKASGGTFSSYAISW | 121 | QSALTQPASVSGSLGQSVTISCTGSSSDVG |
| | | VRQAPGQGLEWMGGIIPIFGTANYAQKFQGRVTITA | | SYNLVSWYQQHPGKAPNLMIYDVSKRSGVS |
| | | DESTSTAYMELSSLRSEDTAVYYCARAPYYYYYMDV | | NRFSGSKSGNTASLTISGLQAEDEADYYCS |
| | | WGQGTTVTVSS | | SYTGISTVVFGGGTKLTVL |
| US10058609B2 | 122 | QMQLVQSGAEVKKPGSSVKVSCKASGGTFSSYAYSW | 123 | SYELMQPPSVSVAPGKTATIACGGENIGRK |
| | | VRQAPGQGLEWMGGIIPSFGTANYAQKFQGRVTITA | | TVHWYQQKPGQAPVLVIYYDSDRPSGIPER |
| | | DESTSTAYMELSSLRSEDTAVYYCARGPIVATITPL | | FSGSNSGNTATLTISRVEAGDEADYYCLVW |
| | | DYWGQGTLVTVSS | | DSSSDHRIFGGGTKLTVL |
| US10517949B2 | 124 | QVQLQQSGAEVKKPGSSVKVSCKASGGTFSSYAISW | 125 | QSVLTQPPSVSAAPGQRVSISCSGRSSNIA |
| | | VRQAPGQGLEWMGRIIPILGIANYAQKFQGRVTITA | | SHDVFWYQQLPGTAPKIVMYETNKRPWGIP |
| | | DKSTSTAYMELSSLRSEDTAVYYCARGGSYGSLYAF | | DRFSGSKSGTSATLDIAGLQTGDEADYYCG |
| | | DIWGQGTMVTVSS | | AWDSGLTGMLFGGGTKVTVL |
| US10517949B2 | 126 | QVQLQQSGAEVKKPGSSVKVSCKASGGTFSSYAISW | 127 | SYELTQPPSVSVAPGKTTRITCGGDNIGRK |
| | | VRQAPGQGLEWMGRIIPILGIANYAQKFQGRVTITA | | SVHWYQQRPGQAPLLLVYDDGDRPSGIPDR |
| | | DKSTSTAYMELSSLRSEDTAVYYCARGGSYGSLYAF | | FSGSNSGNTATLTISGTQAMDEADYYCQAW |
| | | DIWGQGTTVTVSS | | DSTVVFGGGTRLTVL |
| US10517949B2 | 128 | QVQLQQSGAEVKKPGSSVKVSCKASGGTFSSYAISW | 129 | QSVVTQPPSVSAAPGQKVTISCSGSSSNIG |
| | | VRQAPGQGLEWMGRIIPILGIANYAQKFQGRVTITA | | NNYVSWYQQLPGTAPKLLIYDNNERLSGIP |
| | | DKSTSTAYMELSSLRSEDTAVYYCARGGYGGNSLYA | | DRFSGSKSGTSATLGISGLQTGDEADYYCG |
| | | FDIWGQGTMVTVSS | | TWDSSLSVVVFGGGTKLTVL |
| US10517949B2 | 130 | EVQLVQSGAEVKKPGASVKVSCKASGGTFSSYAISW | 131 | QSVLTQPPSVSGAPGQRVTISCTGSSSNIG |
| | | VRQAPGQGLEWMGRIIPILGIANYAQKFQGRVTITA | | AGYDVHWYQQLPGTAPKLLIYGNSNRPSGV |
| | | DKSTSTAYMELSSLRSEDTAVYYCARSGHGYSYGAF | | PDRFSGSKSGTSASLAITGLQAEDEADYYC |
| | | DYWGQGTL | | QSYDSSLSGSYVVFGGGTKLTVL |
| US10517949B2 | 132 | QVQLVQSGAEVKKPGSSVKVSCKASGGTFSSYAISW | 133 | QSVLTQPPSVSGAPGQRVTISCTGSSSNIG |
| | | VRQAPGQGLEWMGRIIPILGIANYAQKFQGRVTITA | | AGYDVHWYQQLPGTAPKLLIYGNSNRPSGV |
| | | DKSTSTAYMELSSLRSEDTAVYYCARSGHGYSYGAF | | PDRFSGSKSGTSASLAITGLQAEDEADYYC |
| | | DYWGQGTLVTVSS | | QSYDSSLSGSYVVFGGGTKLTVL |
| US10544225B2 | 134 | QVQLKESGPGLVAPSQSLSITCTVSGFSLTSYGVHW | 135 | DILMTQSHKFMSTSVGDTVSITCKASQDVG |
| | | VRQPPGKGLEWLGVIWAGGSTNYNSALMSRLSISKD | | IVVAWYQQKPGQSPKLLIYWASIRHTGVPD |
| | | NSKSQVVLKMNSLQTDDTAMYYCAKPYGNSAMDYWG | | RFTGSGSGTDFTLTISNVQSEDLADYFCQQ |
| | | QGTSVTVSS | | YSNYPLYTFGGGTKLEIK |
| US10544225B2 | 136 | EVQLVESGGGLVQPGGSLRLSCAVSGFSLTSYGVHW | 137 | DIQMTQSPSTLSASVGDRVTITCKASQDVG |
| | | VRQAPGKGLEWVAVIWAGGSTNYNSALMSRLTISKD | | IVVAWYQQKPGKAPKLLIYWASIRHTGVPS |
| | | NAKNSVYLQMNSLRAEDTAVYYCAKPYGNSAMDYWG | | RFSGSGSGTEFTLTISSLQPDDFATYYCQQ |
| | | QGTLVTVSS | | YSNYPLYTFGQGTKLEIK |
| US10544225B2 | 138 | EVQLVESGGGLVQPGGSLRLSCAVSGFSLTSYGVHW | 139 | DIQMTQSPSTLSASVGDRVTITCKASQDVG |
| | | VRQAPGKGLEWVAVIWAGGSTNYVDSVKGRFTISKD | | IVVAWYQQKPGKAPKLLIYWASIRHTGVPS |
| | | NAKNSVYLQMNSLRAEDTAVYYCAKPYGNSAMDYWG | | RFSGSGSGTEFTLTISSLQPDDFATYYCQQ |
| | | QGTLVTVSS | | YSNYPLYTFGQGTKLEIK |
| US10544225B2 | 140 | EVQLVESGGGLVQPGGSLRLSCAVSGFSLTSYGVHW | 141 | DIQMTQSPSSLSASVGDRVTITCKASQDVG |
| | | VRQAPGKGLEWVAVIWAGGSTNYVDSVKGRLTISKD | | IVVAWYQQKPGKAPKLLIYWASIRHTGVPS |
| | | NAKNTVYLQMNSLRAEDTAVYYCAKPYGNSAMDYWG | | RFSGSGSGTEFTLTISSLQPDDFATYYCQQ |
| | | QGTLVTVSS | | YSNYPLYTFGQGTKVEIK |
| US10544225B2 | 142 | EVQLVESGGGLVQPGGSLRLSCAVSGFSLTSYGVHW | 143 | DIQMTQSPSSLSASVGDRVTITCKASQDVG |
| | | VRQAPGKGLEWVAVIWAGGSTNYADSVKGRFTISKD | | IVVAWYQQKPGKAPKLLIYWASIRHTGVPS |
| | | NSKNTVYLQMNSLRAEDTAVYYCAKPYGNSAMDYWG | | RFSGSGSGTEFTLTISSLQPDDFATYYCQQ |
| | | QGTLVTVSS | | YSNYPLYTFGQGTKVEIK |
| US10544225B2 | 144 | EVQLVESGGGLVQPGGSLRLSCAVSGFSLTSYGVHW | 145 | DIQMTQSPSSLSASVGDRVTITCKASQDVG |
| | | VRQAPGKGLEWVAVIWAGGSTNYADSVKGRFTISKD | | IVVAWYQQKPGKAPKLLIYWASIRHTGVPS |
| | | TSKNTVYLQMNSLRAEDTAVYYCAKPYGNSAMDYWG | | RFSGSGSGTEFTLTISSLQPDDFATYYCQQ |
| | | QGTLVTVSS | | YSNYPLYTFGQGTKVETK |
| US10544225B2 | 146 | EVQLVESGGGLVQPGGSLRLSCAVSGFSLTSYGVHW | 147 | DIQMTQSPSSLSASVGDRVTITCKASQDVG |
| | | VRQAPGKGLEWVAVIWAGGSTNYVDSVKGRFTISKD | | IVVAWYQQKPGKAPKLLIYWASIRHTGVPS |
| | | NAKNTVYLQMNSLRAEDTAVYYCAKPYGTSAMDYWG | | RFSGSGSGTEFTLTISSLQPDDFATYYCQQ |
| | | QGTLVTVSS | | YSNYPLYTFGQGTKVEIK |
| US10544225B2 | 148 | EVQLVESGGGLVQPGGSLRLSCAVSGFSLTSYGVHW | 149 | DIQMTQSPSSLSASVGDRVTITCKASQDVG |
| | | VRQAPGKGLEWVAVIWAGGSTNYADSVKGRFTISKD | | IVVAWYQQKPGKAPKLLIYWASIRHTGVPS |
| | | TSKNTVYLQMNSLRAEDTAVYYCAKPYGTSAMDYWG | | RFSGSGSGTEFTLTISSLQPDDFATYYCQQ |
| | | QGTLVTVSS | | YSNYPLYTFGQGTKVEIK |
| US10435470B2 | 150 | EVQLVESGGGLVKPGGSLRLSCAASGFIFRSYGMSW | 151 | DIVLTQSPASLAVSPGQRATITCRASQSVS |
| | | VRQAPGKGLEWVASISSGGSTYYPDSVKGRFTISRD | | TSSSSFMHWYQQKPGQPPKLLIKYASNLES |
| | | NAKNSLYLQMNSLRAEDTAVYDCARGYDSGFAYWGQ | | GVPARFSGSGSGTDFTLTINPVEANDTANY |
| | | GTLVTVSS | | YCQHSWEIPYTFGQGTKLEIK |
| US10435470B2 | 152 | QITLKESGPTLVKPTQTLTLTCTVSGFSLSTYGVHW | 153 | DIQMTQSPSSLSASVGDRVTITCKASQSVS |
| | | IRQPPGKALEWLGVIWRGVTTDYNAAFMSRLTITKD | | NDVAWYQQKPGKAPKLLIYYAANRYTGVPD |
| | | NSKNQWLTMNNMDPVDTATYYCARLGFYAMDYWGQG | | RFSGSGYGTDFTFTISSLQPEDIATYFCQQ |
| | | TLVTVSS | | DYTSPYTFGQGTKLEIK |
| US10336824B2 | 154 | EVQLLESGGGLVQPGGSLRLSCAASGFTFSSYAMSW | 155 | DIQMTQSPSSLSASVGDRVTITCRASQSIS |
| | | VRQAPGKGLEWVSSIWRNGIVTVYADSVKGRFTISR | | SYLNWYQQKPGKAPKLLIYAASSLQSGVPS |
| | | DNSKNTLYLQMNSLRAEDTAVYYCAKWSAAFDYWGQ | | RFSGSGSGTDFTLTISSLQPEDFATYYCQQ |
| | | GTLVTVSS | | DNGYPSTFGGGTKVEIKR |
| US20190077867A1 | 156 | EVQLVQSGAEVKKPGASVKVSCKASGYTFTKYIIHW | 157 | DIQLTQSPSFLSASVGDRVTITCRASSSVS |
| | | VRQAPGQGLEWMGWFYPGSGNIRYNEKIKGRVTMTR | | NIHWYQQKPGKAPKPWIYATSNLASGVPSR |
| | | DTSTSTVYMELSSLRSEDTAVYYCARHGELGGGYFF | | FSGSGSGTEFTLTISSLQPEDFATYYCQQW |
| | | DYWGQGTTVTVSS | | SSNPLTFGQGTKLEIKR |
| US20190077867A1 | 158 | EVQLVQSGAEVKKPGASVKVSCKASGYTFTKYIIHW | 159 | DIQLTQSPSFLSASVGDRVTITCRASSKTG |
| | | VRQAPGQGLEWMGWFYPGSGNIRYNEKIKGRVTMTR | | NIHWYQQKPGKAPKPWIYATSNLASGVPSR |
| | | DTSTSTVYMELSSLRSEDTAVYYCARHGELGGGYFF | | FSGSGSGTEFTLTISSLQPEDFATYYCQQW |
| | | DYWGQGTTVTVSS | | SSNPLTFGQGTKLEIKR |
| US20190077867A1 | 160 | EVQLVQSGAEVKKPGASVKVSCKASGYTFTKYIIHW | 161 | DIQLTQSPSFLSASVGDRVTITCRASSGAS |
| | | VRQAPGQGLEWMGWFYPGSGNIRYNEKIKGRVTMTR | | NIHWYQQKPGKAPKPWIYATSNLASGVPSR |
| | | DTSTSTVYMELSSLRSEDTAVYYCARHGELGGGYFF | | FSGSGSGTEFTLTISSLQPEDFATYYCQQW |
| | | DYWGQGTTVTVSS | | SSNPLTFGQGTKLEIKR |
| US9567399B1 | 162 | EVQLVESGGGLVQPGRSLRLSCAASGFTFDDYAMHW | 163 | DIQMTQSPSSLSASVGDRVTITCRASQSIS |
| | | VRQVPGKGLEWVSGISWIRTGIGYADSVKGRFTIFR | | SYLNWYQQKPGKAPKLLIYVASSLQSGVPS |
| | | DNAKNSLYLQMNSLRAEDTALYYCAKDMKGSGTYGG | | RFSGSGSGTQFTLTISSLQPEDFATYYCQQ |
| | | WFDTWGQGTLVTVSS | | SYSTPITFGQGTRLEIK |
| US10214586B2 | 164 | QVQLVQSGAEVKKPGSSVKVSCKASGGTFSSYAISW | 165 | QSVLTQPPSASGTPGQRVTISCSGSSSNIG |
| | | VRQAPGQGLEWMGGIIPIFGTANYAQKFQGRVTITA | | SNTVNWYQQLPGTAPKLLIYGNSNRPSGVP |
| | | DKSTSTAYMELSSLRSEDTAVYYCARSPDYSPYYYY | | DRFSGSKSGTSASLAISGLQSEDEADYYCQ |
| | | GMDVWGQGTTVTVSS | | SYDSSLSGSVFGGGIKLTVLG |
| US20180334504A1 | 166 | QVQLQESGPGLVKPSQTLSLTCTVSGGSISNDYWTW | 167 | DIVMTQSPDSLAVSLGERATINCKSSQSLF |
| | | IRQHPGKGLEYIGYISYTGSTYYNPSLKSRVTISRD | | YHSNQKHSLAWYQQKPGQPPKLLIYGASTR |
| | | TSKNQFSLKLSSVTAADTAVYYCARSGGWLAPFDYW | | ESGVPDRFSGSGSGTQFTLTISSLQAEDVA |
| | | GRGTLVTVSS | | VYYCQQYYGYPYTFGGGTKVEIK |
| US20180334504A1 | 168 | QVQLVQSGAEVKKPGASVKVSCKASGYTFTSYWMHW | 169 | DIVLTQSPASLAVSPGQRATITCRASESVS |
| | | VRQAPGQGLEWMGRIGPNSGFTSYNEKFKNRVTMTR | | IHGTHLMHWYQQKPGQPPKLLIYAASNLES |
| | | DTSTSTVYMELSSLRSEDTAVYYCARGGSSYDYFDY | | GVPARFSGSGSGTDFTLTINPVEAEDTANY |
| | | WGQGTTVTVSS | | YCQQSFEDPLTFGQGTKLEIK |
| US20180346571A1 | 170 | QVQLVQSGAEVKKPGASVKVSCKASGYTFTSYWMHW | 171 | DIQMTQSPSSLSASVGDRVTITCRASQDIG |
| | | VRQAPGQGLEWMGAIYPGNSDTSYNQKFKGRVTMTR | | SSLNWYQQKPGKAPKRLIYATSSLDSGVPS |
| | | DTSTSTVYMELSSLRSEDTAVYYCTRWGYGFDGAMD | | RFSGSGSGTEFTLTISSLQPEDFATYYCLQ |
| | | YWGQGTLVTVSS | | YASSPYTFGGGTKVEIKR |
| US20190010233A1 | 172 | EVQLVESGGGLVQPGGSLRLSCAASGFTFSSYWMSW | 173 | DIQMTQSPSTLSASVGDRVTITCRASQSIS |
| | | VRQAPGKGLEWVADIKQDGSEKYYVDSVKGRFTISR | | SWLAWYQQKPGKAPKLLIYKASSLESGVPS |
| | | DNAKNSLYLQMNSLRAEDTAVYYCARDRLWFGGVMD | | RFSGSGSGTEFTLTISSLQPDDFATYYCQQ |
| | | AWGQGASVTVSS | | YNSYSWTFGQGTKVEIK |
| US20190010233A1 | 174 | EVQLVESGGGLVQPGGSLRLSCAASGITFSSYWMSW | 175 | DIQMTQSPSTLSASVGDRVTITCRASQSIS |
| | | VRQTPGKGLEWVANIKQDGSEKYYVKSVKGRFTISR | | NWLAWYQQKPGKAPKLLIYKASSLESGVPS |
| | | DNAKNSLYLQMNSLRAEDTAVYYCVRDRAVPGTGAF | | RFSGSGSGTEFTLTISSLQPDDFATYYCQQ |
| | | DIWGQGTMVIVSS | | YDSSLFTFGQGTKLEIK |
| US20190010233A1 | 176 | EVQLVESGGGLVQPGGSLRLSCAASGFTFSSYWMSW | 177 | DIQMTQSPSSLSASVGDRVTITCRASQSIS |
| | | VRQAPGKGLEWVANIKQDGNEKYYVDSVKGRFTISR | | SYLNWYQQKPGKAPKLLIYAASSLQSGVPS |
| | | DNAKNSLYLQMNSLRAEDTAVYYCARNIKWGDAFDI | | RFSGSGSGTQFTLTISSLQPEDFAIYSCQQ |
| | | WGQGTMVTVSS | | SYSIPLTFGGGTKVEIK |
| US20190010233A1 | 178 | EVQLVESGGGLVQPGGSLRLSCAASGFTFNTYWMNW | 179 | DIQMTQSPSTLSASVGDRVTITCRASQSIS |
| | | VRQAPGRGLEWVANIKQDGGEKYYVDSVKGRFTISR | | TWLAWYQQKLGKAPKLLIYEVSILESGVPS |
| | | DNAKNSLYLQMNSLRAEDTAMYYCARDGRSWYPDAF | | RFSGSGSGTEFTLTISSLQPDDFATYYCQQ |
| | | DIWGQGTVVTVSS | | YHSFSSFGGGTRVEIK |
| US20190010233A1 | 180 | EVQLVESGGGLVQPGGSLRLSCVVSGFTFSSYWMSW | 181 | DIQMTQSPSTLSASVGDRVTITCRASQSIS |
| | | VRQTPGKGLEWVANIKQDGSEKYYVDSVKGRLTISR | | SWLAWYQQKPGKAPKLLIYKASSLESGVPS |
| | | DNAKNSLYLQMNSLRAEDTAVYYCVRDRAVPGTGAL | | RFSGSGSGTEFTLTISSLQPDDFATYYCQQ |
| | | DIWGQGTMVTVSS | | YNSYLFTFGQGTKLEIK |
| US20190010233A1 | 182 | EVQLVESGGGLVQPGGSLRLSCVASGFTFSSYWMSW | 183 | DIQMTQSPSSLSASVGDRVTITCRASQSIS |
| | | VRQAPGKGLEWVANIKQDGSEKYYVDSVKGRLTISR | | NYLNWYQQKPGKAPKLLIYAASSLQSGVPS |
| | | DNAKNSLYLQMKSLRAEDTAVYYCARDPIYSSYFDY | | RFSGSGSGTDFTLTISSLQPEDFAAYYCQQ |
| | | WGQGILVTVSS | | SYGIPFTFGPGTKVDIK |
| US20190010233A1 | 184 | EVQLVESGGGLVQPGGSLRLSCAASGFTFSSYWMSW | 185 | DIQMTQSPSTLSASEGDRVTITCRASQSIS |
| | | VRQAPGKGLEWVADIKQDGSEKYYVDSVKGRFTISR | | RWLAWYQQKPGKAPKLLIYKASSLESGVPS |
| | | DNAKNSLYLQMNSLRAEDTAVYYCTRDKPHWGGVMD | | RFSGSGSGTEFTLTISSLQPDDFATYYCQQ |
| | | AWGQGTSVTVSS | | YHSYSWTFGQGTKVEIK |
| US20190055312A1 | 186 | EVQLVESGGGLVQPGGSLRLSCTVSGIDLSSYTMGW | 187 | EIVMTQSPSTLSASVGDRVIITCQASEDIY |
| | | VRQAPGKGLEWVGIISSGGRTYYASWAKGRFTISRD | | SLLAWYQQKPGKAPKLLIYDASDLASGVPS |
| | | TSKNTVYLQMNSLRAEDTAVYYCARGRYTGYPYYFA | | RFSGSGSGAEFTLTISSLQPDDFATYYCQG |
| | | LWGQGTLVTVSS | | NYGSSSSSSYGAVFGQGTKLEIK |
| US10465014B2 | 188 | QVQLKESGPGLVAPSQNLSITCTVSGFSLSNYDISW | 189 | DILLTQSPAILSVSPGERVSLSCRASQSIG |
| | | IRQPPGKGLEWLGVIWTGGATNYNSAFMSRLSISRD | | TNIHWFQQRTNGSPRLLIKYASESISGIPS |
| | | NSKSQVFLKMNSLQTQDTAIYYCVRDSNYRYDEPFT | | RFSGSGSGTDFTLSINSVESEDIADYYCQQ |
| | | YWGQGTLVTVSA | | SNSWPYTFGGGTKLEI |
| US10465014B2 | 190 | QVQLQESGPGLVKPSENLSITCTVSGFSLSNYDISW | 191 | EIVLTQSPDTLSVTPKEKVTLTCRASQSIG |
| | | IRQPPGKGLEWLGVIWTGGATNYNPAFKSRLTISRD | | TNIHWFQQRPGQSPKLLIKYASESISGIPS |
| | | NSKSQVSLKMSSLQAADTAVYYCVRDSNYRYDEPFT | | RFSGSGSGTDFTLTINSVEAEDAATYYCQQ |
| | | YWGQGTLVTVSS | | SNSWPYTFGQGTKLEIK |
| US10465014B2 | 192 | QVQLQESGPGLVKPSETLSITCTVSGFSLSNYDISW | 193 | EIVLTQSPDTLSVTPKEKVTLTCRASQSIG |
| | | IRQPPGKGLEWLGVIWTGGATNYNPALKSRLTISRD | | TNIHWFQQKPGQSPKLLIKYASESISGVPS |
| | | NSKNQVSLKMSSVTAADTAVYYCVRDSNYRYDEPFT | | RFSGSGSGTDFTLTINSVEAEDAATYYCQQ |
| | | YWGQGTLVTVSS | | SNSWPYTFGQGTKLEIK |
| US10208119B2 | 194 | EVQLVESGGGLVQPGGSLRLSCAASGFTFSSYDMSW | 195 | DIQMTQSPSSLSASVGDRVTITCKASQDVT |
| | | VRQAPGKSLEWVATISDAGGYIYYSDSVKGRFTISR | | PAVAWYQQKPGKAPKLLIYSTSSRYTGVPS |
| | | DNAKNSLYLQMNSLRDEDTAVYICAREFGKRYALDY | | RFSGSGSGTDFTFTISSLQPEDIATYYCQQ |
| | | WGQGTTVTVSS | | HYTTPLTFGQGTKLEIK |
| US10590199B2 | 196 | EVQLVQSGAEVKKPGSSVKVSCKASGGTFSRSAISW | 197 | NFMLTQPHSVSESPGKTVTISCTRSSGSID |
| | | VRQAPGQGLEWMGVIIPAFGEANYAQKFQGRVTITA | | SNYVQWYQQRPGSAPTTVIYEDNQRPSGVP |
| | | DESTSTAYMELSSLRSEDTAVYYCARGRQMFGAGID | | DRFSGSIDSSSNSASLTISGLKTEDEADYY |
| | | FWGQGTLVTVSS | | CQSYDSNNRHVIFGGGTKLTVL |
| WO2018194496 | 198 | EVQLVESGGGVVRPGGSLRLSCAASGFTFDDYAMSW | 199 | QTVVTQEPSLSVSPGGTVTLTCGLSSGTVT |
| | | VRQAPGKGLEWVSDISWSGSNTNYADSVKGRFTISR | | AINYPGWYQQTPGQAPRTLIYNTNTRHSGV |
| | | DNAKNSLYLQMNSLRAEDTALYHCARAPLLLAMTFG | | PDRFSGSISGNKAALTITGAQAEDEADYYC |
| | | VGSWGQGTLVTVSS | | ALYMGNGGHMFGGGTK |
| US9580507B2 | 200 | QVQLVQSGAEVKKPGASVKVSCKASGYTFTDYGFSW | 201 | EIVLTQSPATLSLSPGERATLSCRASQSVS |
| | | VRQAPGQGLEWMGWITAYNGNTNYAQKLQGRVTMTT | | SYLVWYQQKPGQAPRLLIYDASNRATGIPA |
| | | DTSTSTVYMELRSLRSDDTAVYYCARDYFYGMDVWG | | RFSGSGSGTDFTLTISSLEPEDFAVYYCQQ |
| | | QGTTVTVSS | | RSNWPRTFGQGTKVEIK |
| US9580507B2 | 202 | QVQLVQSGAEVKKPGSSVKVSCKTSGDTFSTYAISW | 203 | EIVLTQSPATLSLSPGERATLSCRASQSVS |
| | | VRQAPGQGLEWMGGIIPIFGKAHYAQKFQGRVTITA | | SYLAWYQQKPGQAPRLLIYDASNRATGIPA |
| | | DESTSTAYMELSSLRSEDTAVYFCARKFHFVSGSPF | | RFSGSGSGTDFTLTISSLEPEDFAVYYCQQ |
| | | GMDVWGQGTTVTVSS | | RSNWPTFGQGTKVEIK |
| US9580507B2 | 204 | QVQLVQSGAEVKKPGASVKVSCKASGYTFTSYDVHW | 205 | DIQMTQSPSSLSASVGDRVTITCRASQGIS |
| | | VRQAPGQRLEWMGWLHADTGITKFSQKFQGRVTITR | | SWLAWYQQKPEKAPKSLIYAASSLQSGVPS |
| | | DTSASTAYMELSSLRSEDTAVYYCARERIQLWFDYW | | RFSGSGSGTDFTLTISSLQPEDFATYYCQQ |
| | | GQGTLVTVSS | | YNSYPYTFGQGTKLEIK |
| US9580507B2 | 206 | QVQLVQSGAEVKKPGSSVKVSCKVSGGIFSTYAINW | 207 | EIVLTQSPGTLSLSPGERATLSCRASQSVS |
| | | VRQAPGQGLEWMGGIIPIFGTANHAQKFQGRVTITA | | SSYLAWYQQKPGQAPRLLIYGASSRATGIP |
| | | DESTSTAYMELSSLRSEDTAVYYCARDQGIAAALFD | | DRFSGSGSGTDFTLTISRLEPEDFAVYYCQ |
| | | YWGQGTLVTVSS | | QYGSSPWTFGQGTKVEIK |
| US9580507B2 | 208 | EVQLVESGGGLVQPGRSLRLSCAVSGFTFDDYVVHW | 209 | DIQMTQSPSSLSASVGDRVTITCRASQGIS |
| | | VRQAPGKGLEWVSGISGNSGNIGYADSVKGRFTISR | | SWLAWYQQKPEKAPKSLIYAASSLQSGVPS |
| | | DNAKNSLYLQMNSLRAEDTALYYCAVPFDYWGQGTL | | RFSGSGSGTDFTLTISSLQPEDFATYYCQQ |
| | | VTVSS | | YNSYPYTFGQGTKLEIK |
| US9580507B2 | 210 | QVQLVQSGAEVKKPGSSVKVSCKTSGDTFSSYAISW | 211 | EIVLTQSPATLSLSPGERATLSCRASQSVS |
| | | VRQAPGQGLEWMGGIIPIFGRAHYAQKFQGRVTITA | | SYLAWYQQKPGQAPRLLIYDASNRATGIPA |
| | | DESTSTAYMELSSLRSEDTAVYFCARKFHFVSGSPF | | RFSGSGSGTDFTLTISSLEPEDFAVYYCQQ |
| | | GMDVWGQGTTVTVSS | | RSNWPTFGQGTKVEIK |
| US9580507B2 | 212 | QVQLVQSGAEVKKPGSSVKVSCKTSGGTFSSYAISW | 213 | EIVLTQSPATLSLSPGERATLSCRASQSVS |
| | | VRQAPGQGLEWMGGIIPIFGKAHYAQKFQGRVTITA | | SYLAWYQQKPGQAPRLLIYDASNRATGIPA |
| | | DESTTTAYMELSSLRSEDTAVYYCARKYDYVSGSPF | | RFSGSGSGTDFTLTISSLEPEDFAVYYCQQ |
| | | GMDVWGQGTTVTVSS | | RSNWPTFGQGTKVEIK |
| US9580507B2 | 214 | QVQLVQSGAEVKKPGSSVKVSCKASGGTFSSYAINW | 215 | EIVLTQSPGTLSLSPGERATLSCRASQSVS |
| | | VRQAPGQGLEWMGGIIPIFGSANYAQKFQDRVTITA | | SSYLAWYQQKPGQAPRLLIYGASSRATGIP |
| | | DESTSAAYMELSSLRSEDTAVYYCARDSSGWSRYYM | | DRFSGSGSGTDFTLTISRLEPEDFAVYYCQ |
| | | DVWGQGTTVTVSS | | QYGSSPFGGGTKVEIK |
| US9580507B2 | 216 | QVQLVQSGAEVKEPGSSVKVSCKASGGTFNSYAISW | 217 | EIVLTQSPATLSLSPGERATLSCRASQSVS |
| | | VRQAPGQGLEWMGGIIPLFGIAHYAQKFQGRVTITA | | SYLAWYQQKPGQAPRLLIYDASNRATGIPA |
| | | DESTNTAYMDLSSLRSEDTAVYYCARKYSYVSGSPF | | RFSGSGSGTDFTLTISSLEPEDFAVYYCQQ |
| | | GMDVWGQGTTVTVSS | | RSNWPTFGQGTRLEIK |
| US9580507B2 | 218 | EVQLVESGGGLVQPGRSLRLSCAASGITFDDYGMHW | 219 | AIQLTQSPSSLSASVGDRVTITCRASQGIS |
| | | VRQAPGKGLEWVSGISWNRGRIEYADSVKGRFTISR | | SALAWYQQKPGKAPKLLIYDASSLESGVPS |
| | | DNAKNSLYLQMNSLRAEDTALYYCAKGRFRYFDWFL | | RFSGSGSGTDFTLTISSLQPEDFATYYCQQ |
| | | DYWGQGTLVTVSS | | FNSYPFTFGPGTKVDIK |
| US8779108B2 | 220 | EVQLVESGGGLVQPGGSLRLSCAASGFTFSRYWMSW | 221 | EIVLTQSPGTLSLSPGERATLSCRASQRVS |
| | | VRQAPGKGLEWVANIKQDGSEKYYVDSVKGRFTISR | | SSYLAWYQQKPGQAPRLLIYDASSRATGIP |
| | | DNAKNSLYLQMNSLRAEDTAVYYCAREGGWFGELAF | | DRFSGSGSGTDFTLTISRLEPEDFAVYYCQ |
| | | DYWGQGTLVTVSS | | QYGSLPWTFGQGTKVEIK |
| US9624298B2 | 222 | EVQLLESGGGLVQPGGSLRLSCAASGFTFSSYIMMW | 223 | QSALTQPASVSGSPGQSITISCTGTSSDVG |
| | | VRQAPGKGLEWVSSIYPSGGITFYADTVKGRFTISR | | AYNYVSWYQQHPGKAPKLMIYDVSNRPSGV |
| | | DNSKNTLYLQMNSLRAEDTAVYYCARIKLGTVTTVD | | SNRFSGSKSGNTASLTISGLQAEDEADYYC |
| | | YWGQGTLVTVSS | | SSYTSSSTRVFGTGTKVTVL |

What is claimed is:

1. A multimeric binding molecule comprising two or five bivalent binding units or multimerizing variants or fragments thereof and a modified J-chain,
wherein each binding unit comprises two IgA or IgM heavy chain constant regions or multimerizing variants or fragments thereof, each associated with an antigen-binding domain for a total of four or ten antigen-binding domains,
wherein at least three of the antigen-binding domains of the binding molecule specifically bind to a target antigen, and
wherein the modified J-chain comprises (a) a J-chain or functional fragment or variant thereof (“J”), and (b) an immunostimulatory agent (“ISA”), wherein J and the ISA are associated as a fusion protein.

2. The multimeric binding molecule of claim 1, wherein the ISA comprises a cytokine or receptor-binding fragment or variant thereof.

3. The multimeric binding molecule of claim 2, wherein the cytokine or fragment or variant thereof comprises IL-15 or IL-2, or a receptor-binding fragment or variant thereof.

4. The multimeric binding molecule of any one of claims 1 to 3, wherein the ISA comprises (a) an interleukin-15 (IL-15) protein or receptor-binding fragment or variant thereof (“I”), and (b) an interleukin-15 receptor-α (IL-15Rα) fragment comprising the sushi domain or a variant thereof capable of associating with I (“R”), wherein J and at least one of I and R are associated as a fusion protein, and wherein I and R can associate to function as the ISA.

5. The multimeric binding molecule of any one of claims 1 to 4, wherein J is a wild-type human J-chain and comprises the amino acid sequence SEQ ID NO: 2 or a functional fragment or variant thereof.

6. The multimeric binding molecule of any one of claims 1 to 5, wherein J is a variant J-chain or fragment thereof comprising one or more single amino acid substitutions, deletions, or insertions relative to a wild-type J-chain that can affect serum half-life of the multimeric binding molecule; and wherein the multimeric binding molecule exhibits an increased serum half-life upon administration to an animal relative to a reference multimeric binding molecule that is identical except for the one or more single amino acid substitutions, deletions, r insertions, and is administered in the same way to the same animal species.

7. The multimeric binding molecule of claim 6, wherein the J comprises an amino acid substitution at the amino acid position corresponding to amino acid Y102 of the mature wild-type human J-chain (SEQ ID NO: 2).

8. The multimeric binding molecule of claim 7, wherein the amino acid corresponding to Y102 of SEQ ID NO: 2 is substituted with alanine (A), serine (S), or arginine (R).

9. The multimeric binding molecule of claim 8, wherein the amino acid corresponding to Y102 of SEQ ID NO: 2 is substituted with alanine (A).

10. The multimeric binding molecule of claim 9, wherein J is a variant human J-chain and comprises the amino acid sequence SEQ ID NO: 3 (“J*”) or amino acids 1-137 of SEQ ID NO: 86.

11. The multimeric binding molecule of any one of claims 1 to 10, wherein J is a variant J-chain or fragment thereof comprising one or more single amino acid substitutions, deletions, or insertions relative to a wild-type J-chain that reduces glycosylation of the J-chain.

12. The multimeric binding molecule of claim 11, wherein the J comprises an amino acid substitution at the amino acid position corresponding to amino acid N49 of the mature wild-type human J-chain (SEQ ID NO: 2).

13. The multimeric binding molecule of claim 12, wherein the amino acid corresponding to N49 of SEQ ID NO: 2 is substituted with aspartic acid (D).

14. The multimeric binding molecule of any one of claims 4 to 13, wherein I comprises the mature human IL-15 amino acid sequence of SEQ ID NO: 4 or a receptor-binding variant or fragment thereof.

15. The multimeric binding molecule of claim 14, wherein the receptor-binding variant comprises at least one, but no more than ten, single amino acid insertions, deletions, or substitutions, and wherein the single amino acid insertions, deletions, or substitutions reduce the affinity of the IL-15 variant for its receptor.

16. The multimeric binding molecule of claim 15, wherein I comprises one, two, three, four, five, six, seven, or eight amino acid substitutions.

17. The multimeric binding molecule of claim 16, wherein the amino acid substitutions are at one or more of positions corresponding to N1, N4, D8, D30, D61, E64, N65, N72, or Q108 of SEQ ID NO: 4.

18. The multimeric binding molecule of claim 17, wherein the amino acid substitutions comprise one or more of substitutions N1D, N4D, D8N, D30N, D61N, E64Q, N65D, N72D, or Q108E in SEQ ID NO: 4.

19. The multimeric binding molecule of claim 18, wherein I comprises SEQ ID NO: 4 except for:
(a) a single amino acid substitution at a position selected from the group consisting of N1D, N4D, D8N, D30N, D61N, E64Q, N65D, N72D, and Q108E;
(b) two amino acid substitutions at positions selected from the group consisting of N4D/N65D and N1D/N65D; or
(c) three amino acid substitutions at positions D30N/E64Q/N65D.

20. The multimeric binding molecule of claim 19, wherein I comprises the amino acid sequence SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, or SEQ ID NO: 68.

21. The multimeric binding molecule of claim 14, wherein the receptor-binding variant comprises at least one, but no more than ten, single amino acid insertions, deletions, or substitutions, and wherein the single amino acid insertions, deletions, or substitutions reduce the glycosylation of the IL-15 variant.

22. The multimeric binding molecule of claim 21, wherein I comprises one, two, three, four, five, six, seven, or eight amino acid substitutions.

23. The multimeric binding molecule of claim 22, wherein the amino acid substitutions are at one or more of positions corresponding to N71, S73, N79, or N112 of SEQ ID NO: 4.

24. The multimeric binding molecule of claim 23, wherein the amino acid substitutions comprise one or more of substitutions N71D, S73I, N79D, or N112D in SEQ ID NO: 4.

25. The multimeric binding molecule of claim 24, wherein I comprises SEQ ID NO: 4 except for one or more amino acid substitutions at a position selected from the group consisting of N71D, S73I, N79D, and N112D.

26. The multimeric binding molecule of claim 25, wherein I comprises the amino acid sequence of amino acids 246-361 of SEQ ID NO: 85, SEQ ID NO: 87, SEQ ID NO: 88, SEQ ID NO: 89, or SEQ ID NO: 90.

27. The multimeric binding molecule of any one of claims 4 to 26, wherein R comprises the amino acid sequence SEQ ID NO: 5 or a variant or fragment thereof that is capable of associating with human IL-15.

28. The multimeric binding molecule of any one of claims 4 to 20, wherein R consists essentially of or consists of the amino acid sequence SEQ ID NO: 5 or a variant thereof that is capable of associating with human IL-15.

29. The multimeric binding molecule of any one of claims 4 to 28, wherein J and I are associated as a fusion protein.

30. The multimeric binding molecule of any one of claims 4 to 28, wherein J and R are associated as a fusion protein.

31. The multimeric binding molecule of any one of claims 4 to 30, wherein J, I, and R are associated as a fusion protein.

32. The multimeric binding molecule of claim 31, wherein J, I, and R are fused via linkers.

33. The multimeric binding molecule of claim 32, wherein the linkers are the same or different.

34. The multimeric binding molecule of claim 32 or claim 33, wherein at least one linker comprises, consists essentially of, or consists of the amino acid sequence GGGGSGGGGSGGGGS (SEQ ID NO: 78).

35. The multimeric binding molecule of claim 32 or claim 33, wherein at least one linker comprises, consists essentially of, or consists of the amino acid sequence GGSGGGGSGGGSGGGGSLQ (SEQ ID NO: 79).

36. The multimeric binding molecule of any one of claims 6 to 35, wherein J is 1*, and wherein the modified J-chain is arranged from N-terminus to C-terminus as J*-R-I, J-I-R, I-R-J*, R-I-J*, R-J*-I, I-J*-R, I-J*, or J*-I, wherein “-” is a linker.

37. The multimeric binding molecule of claim 36, wherein the modified J-chain comprises the amino acid sequence SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 77, SEQ ID NO: 85, SEQ ID NO: 86, SEQ ID NO: 87, SEQ ID NO: 88, SEQ ID NO: 89, or SEQ ID NO: 90.

38. The multimeric binding molecule of claim 36, wherein the modified J-chain is arranged from N-terminus to C-terminus as J*-R-I.

39. The multimeric binding molecule of claim 38, wherein the modified J-chain comprises the amino acid sequence SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 77, SEQ ID NO: 85, SEQ ID NO: 86, SEQ ID NO: 87, SEQ ID NO: 88, SEQ ID NO: 89, or SEQ ID NO: 90.

40. The multimeric binding molecule of any one of claims 1 to 3, wherein the ISA comprises a variant of human IL-2 (“IL2v”) that does not bind to the α-subunit of the IL-2 receptor.

41. The multimeric binding molecule of claim 40, wherein IL2v comprises the amino acid sequence SEQ ID NO: 31.

42. The multimeric binding molecule of claim 41, wherein the modified J-chain comprises the amino acid sequence SEQ ID NO: 32.

43. The multimeric binding molecule of any one of claims 4 to 42, wherein the modified J-chain further comprises an antigen-binding domain of an antibody fused thereto.

44. The multimeric binding molecule of claim 43, wherein the antigen-binding domain binds to a target on an immune effector cell.

45. The multimeric binding molecule of claim 44, wherein the immune effector cell is a CD8+ T cell.

46. The multimeric binding molecule of claim 45, wherein the antigen binding domain is a single-chain Fv (scFv) antibody fragment that specifically binds to CD3epsilon (CD3ε).

47. The multimeric binding molecule of claim 46, wherein the modified J-chain comprises SEQ ID NO: 19.

48. The multimeric binding molecule of any one of claims 1 to 47, which is pentameric and comprises five binding units, wherein each binding unit comprises two IgM heavy chain constant regions or multimerizing variants or fragments thereof.

49. The multimeric binding molecule of any one of claims 1 to 47, which is dimeric and comprises two binding units or multimerizing variants or fragments thereof, wherein each binding unit comprises two IgA heavy chain constant regions or multimerizing variants or fragments thereof.

50. The multimeric binding molecule of any one of claims 1 to 49, wherein the target antigen comprises a tumor-associated antigen or a target that modulates a T cell response or NK cell response.

51. The multimeric binding molecule of claim 50, wherein the target antigen comprises a target that modulates a T cell response or an NK cell response.

52. The multimeric binding molecule of claim 51, wherein the target inhibits CD8+ T cell or NK cell activity.

53. The multimeric binding molecule of claim 52, wherein the target comprises an inhibitory immune checkpoint protein, and wherein the antigen-binding domains antagonize the target, thereby stimulating CD8+ T cells or NK cells.

54. The multimeric binding molecule of claim 53, wherein the inhibitory immune checkpoint protein comprises a programmed cell death-1 protein (PD-1), a programmed cell death ligand-1 protein (PD-L1), a lymphocyte-activation gene 3 protein (LAG3), a T-cell immunoglobulin and mucin domain 3 protein (TIM3), a cytotoxic T-lymphocyte-associated protein 4 (CTLA4), a B- and T-lymphocyte attenuator protein (BTLA), a V-domain Ig suppressor of T-cell activation protein (VISTA), a T-cell immunoreceptor with Ig and ITIM Domains protein (TIGIT), a Killer-cell Immunoglobulin-like Receptor protein (KIR), a B7-H3 protein, a B7-H4 protein, or any combination thereof.

55. The multimeric binding molecule of claim 54, wherein the inhibitory immune checkpoint protein comprises PD-L1, and wherein the antigen-binding domain comprises a heavy chain variable region (VH) comprising the amino acid sequence SEQ ID NO: 33, SEQ ID NO: 91, SEQ ID NO: 92, or SEQ ID NO: 93 and a light chain variable region (VL) comprising the amino acid sequence SEQ ID NO: 34 or SEQ ID NO: 94.

56. The multimeric binding molecule of claim 54, wherein the inhibitory immune checkpoint protein comprises PD-L1, and wherein the antigen-binding domain comprises a heavy chain variable region (VH) and a light chain variable region (VL) wherein the VH and VL comprise six immunoglobulin complementarity determining regions HCDR1, HCDR2, HCDR3, LCDR1, LCDR2, and LCDR3, wherein the HCDR1, HCDR2, HCDR3, LCDR1, LCDR2, and LCDR3 comprise the CDRs of an antibody comprising the VH and VL of SEQ ID NO: 75 and SEQ ID NO: 76, SEQ ID NO: 96 and SEQ ID NO: 97, SEQ ID NO: 98 and SEQ ID NO: 99, SEQ ID NO: 100 and SEQ ID NO: 101, SEQ ID NO: 102 and SEQ ID NO: 103, SEQ ID NO: 104 and SEQ ID NO: 105, SEQ ID NO: 106 and SEQ ID NO: 107, SEQ ID NO: 108 and SEQ ID NO: 109, SEQ ID NO: 110 and SEQ ID NO: 111, SEQ ID NO: 112 and SEQ ID NO: 113, SEQ ID NO: 114 and SEQ ID NO: 115, SEQ ID NO: 116 and SEQ ID NO: 117, SEQ ID NO: 118 and SEQ ID NO: 119, SEQ ID NO: 120 and SEQ ID NO: 121, SEQ ID NO: 122 and SEQ ID NO: 123, SEQ ID NO: 124 and SEQ ID NO: 125, SEQ ID NO: 126 and SEQ ID NO: 127, SEQ ID NO: 128 and SEQ ID NO: 129, SEQ ID NO: 130 and SEQ ID NO: 131, SEQ ID NO: 132 and SEQ ID NO: 133, SEQ ID NO: 134 and SEQ ID NO: 135, SEQ ID NO: 136 and SEQ ID NO: 137, SEQ ID NO: 138 and SEQ ID NO: 139, SEQ ID NO: 140 and SEQ ID NO: 141, SEQ ID NO: 142 and SEQ ID NO: 143, SEQ ID NO: 144 and SEQ ID NO: 145, SEQ ID NO: 146 and SEQ ID NO: 147, SEQ ID NO: 148 and SEQ ID NO: 149, SEQ ID NO: 150 and SEQ ID NO: 151, SEQ ID NO: 152 and SEQ ID NO: 153, SEQ ID NO: 154 and SEQ ID NO: 155, SEQ ID NO: 156 and SEQ ID NO: 157, SEQ ID NO: 158 and SEQ ID NO: 159, SEQ ID NO: 160 and SEQ ID NO: 161, SEQ ID NO: 162 and SEQ ID NO: 163, SEQ ID NO: 164 and SEQ ID NO: 165, SEQ ID NO: 166 and SEQ ID NO: 167, SEQ ID NO: 168 and SEQ ID NO: 169, SEQ ID NO: 170 and SEQ ID NO: 171, SEQ ID NO: 172 and SEQ ID NO: 173, SEQ ID NO: 174 and SEQ ID NO: 175, SEQ ID NO: 176 and SEQ ID NO: 177, SEQ ID NO: 178 and SEQ ID NO: 179, SEQ ID NO: 180 and SEQ ID NO: 181, SEQ ID NO: 182 and SEQ ID NO: 183, SEQ ID NO: 184 and SEQ ID NO: 185, SEQ ID NO: 186 and SEQ ID NO: 187, SEQ ID NO: 188 and SEQ ID NO: 189, SEQ ID NO: 190 and SEQ ID NO: 191, SEQ ID NO: 192 and SEQ ID NO: 193, SEQ ID NO: 194 and SEQ ID NO: 195, SEQ ID NO: 196 and SEQ ID NO: 197, SEQ ID NO: 198 and SEQ ID NO: 199, SEQ ID NO: 200 and SEQ ID NO: 201, SEQ ID NO: 202 and SEQ ID NO: 203, SEQ ID NO: 204 and SEQ ID NO: 205, SEQ ID NO: 206 and SEQ ID NO: 207, SEQ ID NO: 208 and SEQ ID NO: 209, SEQ ID NO: 210 and SEQ ID NO: 211, SEQ ID NO: 212 and SEQ ID NO: 213, SEQ ID NO: 214 and SEQ ID NO: 215, SEQ ID NO: 216 and SEQ ID NO: 217, SEQ ID NO: 218 and SEQ ID NO: 219, SEQ ID NO: 220 and SEQ ID NO: 221, or SEQ ID NO: 222 and SEQ ID NO: 223, respectively with zero, one, or two single amino acid substitutions in one or more of the HCDRs or LCDRs.

57. The multimeric binding molecule of claim 54 or claim 56, wherein the inhibitory immune checkpoint protein comprises PD-L1, and wherein the antigen-binding domain comprises a heavy chain variable region (VH) and a light chain variable region (VL) wherein the VH and VL comprise amino acid sequences at least 80%, at least 85%, at least 90%, at least 95% or 100% identical to the mature VH and VL amino acid sequences comprising SEQ ID NO: 75 and SEQ ID NO: 76, SEQ ID NO: 96 and SEQ ID NO: 97, SEQ ID NO: 98 and SEQ ID NO: 99, SEQ ID NO: 100 and SEQ ID NO: 101, SEQ ID NO: 102 and SEQ ID NO: 103, SEQ ID NO: 104 and SEQ ID NO: 105, SEQ ID NO: 106 and SEQ ID NO: 107, SEQ ID NO: 108 and SEQ ID NO: 109, SEQ ID NO: 110 and SEQ ID NO: 111, SEQ ID NO: 112 and SEQ ID NO: 113, SEQ ID NO: 114 and SEQ ID NO: 115, SEQ ID NO: 116 and SEQ ID NO: 117, SEQ ID NO: 118 and SEQ ID NO: 119, SEQ ID NO: 120 and SEQ ID NO: 121, SEQ ID NO: 122 and SEQ ID NO: 123, SEQ ID NO: 124 and SEQ ID NO: 125, SEQ ID NO: 126 and SEQ ID NO: 127, SEQ ID NO: 128 and SEQ ID NO: 129, SEQ ID NO: 130 and SEQ ID NO: 131, SEQ ID NO: 132 and SEQ ID NO: 133, SEQ ID NO: 134 and SEQ ID NO: 135, SEQ ID NO: 136 and SEQ ID NO: 137, SEQ ID NO: 138 and SEQ ID NO: 139, SEQ ID NO: 140 and SEQ ID NO: 141, SEQ ID NO: 142 and SEQ ID NO: 143, SEQ ID NO: 144 and SEQ ID NO: 145, SEQ ID NO: 146 and SEQ ID NO: 147, SEQ ID NO: 148 and SEQ ID NO: 149, SEQ ID NO: 150 and SEQ ID NO: 151, SEQ ID NO: 152 and SEQ ID NO: 153, SEQ ID NO: 154 and SEQ ID NO: 155, SEQ ID NO: 156 and SEQ ID NO: 157, SEQ ID NO: 158 and SEQ ID NO: 159, SEQ ID NO: 160 and SEQ ID NO: 161, SEQ ID NO: 162 and SEQ ID NO: 163, SEQ ID NO: 164 and SEQ ID NO: 165, SEQ ID NO: 166 and SEQ ID NO: 167, SEQ ID NO: 168 and SEQ ID NO: 169, SEQ ID NO: 170 and SEQ ID NO: 171, SEQ ID NO: 172 and SEQ ID NO: 173, SEQ ID NO: 174 and SEQ ID NO: 175, SEQ ID NO: 176 and SEQ ID NO: 177, SEQ ID NO: 178 and SEQ ID NO: 179, SEQ ID NO: 180 and SEQ ID NO: 181, SEQ ID NO: 182 and SEQ ID NO: 183, SEQ ID NO: 184 and SEQ ID NO: 185, SEQ ID NO: 186 and SEQ ID NO: 187, SEQ ID NO: 188 and SEQ ID NO: 189, SEQ ID NO: 190 and SEQ ID NO: 191, SEQ ID NO: 192 and SEQ ID NO: 193, SEQ ID NO: 194 and SEQ ID NO: 195, SEQ ID NO: 196 and SEQ ID NO: 197, SEQ ID NO: 198 and SEQ ID NO: 199, SEQ ID NO: 200 and SEQ ID NO: 201, SEQ ID NO: 202 and SEQ ID NO: 203, SEQ ID NO: 204 and SEQ ID NO: 205, SEQ ID NO: 206 and SEQ ID NO: 207, SEQ ID NO: 208 and SEQ ID NO: 209, SEQ ID NO: 210 and SEQ ID NO: 211, SEQ ID NO: 212 and SEQ ID NO: 213, SEQ ID NO: 214 and SEQ ID NO: 215, SEQ ID NO: 216 and SEQ ID NO: 217, SEQ ID NO: 218 and SEQ ID NO: 219, SEQ ID NO: 220 and SEQ ID NO: 221, or SEQ ID NO: 222 and SEQ ID NO: 223, respectively.

58. The multimeric binding molecule of claim 50 or claim 51, wherein the target comprises a TNF receptor superfamily target, and wherein the antigen-binding domains can agonize the target.

59. The multimeric binding molecule of claim 58, wherein the target antigen comprises GITR, OX40, or a combination thereof, and wherein the antigen-binding domains can agonize the target.

60. The multimeric binding molecule of claim 59, wherein the target antigen comprises GITR, and wherein the antigen-binding domain comprises a heavy chain variable region (VH) and a light chain variable region (VL) comprising, respectively, the amino acid sequences SEQ ID NO: 35 and SEQ ID NO: 36, SEQ ID NO: 37 and SEQ ID NO: 38, SEQ ID NO: 39 and SEQ ID NO: 40, SEQ ID NO: 41 and SEQ ID NO: 42, or SEQ ID NO: 43 and SEQ ID NO: 44.

61. The multimeric binding molecule of claim 59, wherein the target antigen comprises OX40, and wherein the antigen-binding domain comprises a heavy chain variable region (VH) and a light chain variable region (VL) comprising, respectively, the amino acid sequences SEQ ID NO: 45 and SEQ ID NO: 46 or SEQ ID NO: 47 and SEQ ID NO: 48.

62. The multimeric binding molecule of any one of claims 1 to 49, wherein the target antigen comprises a tumor-associated antigen.

63. The multimeric binding molecule of claim 62, wherein the tumor associated antigen comprises B-cell maturation antigen (BCMA), CD19, CD20, EGFR, HER2 (ErbB2), ErbB3, ErbB4, CTLA4, PD-1, PD-L1, VEGF, VEGFR1, VEGFR2, CD52, CD30, prostate-specific membrane antigen (PSMA), CD38, GD2, SLAMF7, platelet-derived growth factor receptor A (PDGFRA), CD22, FLT3 (CD135), CD123, MUC-16, carcinoembryonic antigen-related cell adhesion molecule 1 (CEACAM-1), mesothelin, tumor-associated calcium signal transducer 2 (Trop-2), glypican-3 (GPC-3), human blood group H type 1 trisaccharide (Globo-H), sialyl Tn antigen (STn antigen), CD33, or any combination thereof.

64. The multimeric binding molecule of claim 63, wherein the target antigen comprises CD20, and wherein the antigen-binding domain comprises a heavy chain variable region (VH) and a light chain variable region (VL) comprising, respectively, the amino acid sequences SEQ ID NO: 49 and SEQ ID NO: 50.

65. The multimeric binding molecule of any one of claims 1 to 64, wherein at least four, at least five, at least six, at least seven, at least eight, at least nine or ten of the antigen-binding domains of the binding molecule specifically bind to the same target antigen.

66. The multimeric binding molecule of any one of claims 1 to 47 or 49 to 65, wherein each binding unit comprises two IgA heavy chain constant regions or multimerizing fragments or variants thereof, each comprising an IgA Cal domain, an IgA hinge, an IgA Cα2 domain, IgA Cα3 domain, and an IgA tailpiece domain.

67. The multimeric binding molecule of claim 66, wherein the IgA heavy chain constant regions comprise the amino acid sequence SEQ ID NO: 53, SEQ ID NO: 54, or any multimerizing variant or fragment thereof.

68. The multimeric binding molecule of any one of claims 1 to 46 or 48 to 65, wherein each binding unit comprises two IgM heavy chain constant regions or multimerizing fragments or variants thereof, each comprising an IgM Cμ4 domain and an IgM tailpiece domain.

69. The multimeric binding molecule of claim 68, wherein each IgM heavy chain constant region or multimerizing fragment or variant thereof further comprises an IgM Cμ3 domain, an IgM Cμ2 domain, an IgM Cμ1 domain, or any combination thereof, situated N-terminal to the IgM Cμ4 and IgM tailpiece domains.

70. The multimeric binding molecule of claim 68 or claim 69, wherein each IgM heavy chain constant region is a human IgM constant region or multimerizing variant or fragment thereof, comprising the amino acid sequence SEQ ID NO: 51, SEQ ID NO: 52, or a multimerizing variant or fragment thereof.

71. The multimeric binding molecule of claim 69 or 70, comprising a variant human IgM constant region, wherein the multimeric binding molecule has reduced CDC activity relative to a multimeric binding molecule comprising IgM heavy chain constant regions comprising the amino acid sequence SEQ ID NO: 51, SEQ ID NO: 52, or a multimerizing variant or fragment thereof.

72. The multimeric binding molecule of claim 71, wherein each IgM heavy chain constant region comprises a variant of the amino acid sequence SEQ ID NO: 51 or SEQ ID NO: 52, wherein the variant comprises an amino acid substitution at position P311 of SEQ ID NO: 51 or SEQ ID NO: 52, an amino acid substitution at position P313 of SEQ ID NO: 51 or SEQ ID NO: 52, or amino acid substitutions at positions P311 and P313 of SEQ ID NO: 51 or SEQ ID NO: 52.

73. The multimeric binding molecule of claim 69 or 70, wherein each IgM heavy chain constant region is a variant human IgM constant region with one or more single amino acid substitutions, deletions, or insertions relative to a reference IgM heavy chain constant region identical to the variant IgM heavy chain constant regions except for the one or more single amino acid substitutions, deletions, or insertions, and wherein the multimeric binding molecule exhibits increased serum half-life upon administration to a subject animal relative to a multimeric binding molecule comprising the reference IgM heavy chain constant regions, and is administered in the same way to the same animal species.

74. The multimeric binding molecule of claim 73, wherein the variant IgM heavy chain constant regions comprise amino acid substitutions at one or more amino acid positions corresponding to amino acid, E345A, S401A, E402A, or E403A of the wild-type human IgM constant region SEQ ID NO: 51 or SEQ ID NO: 52.

75. An isolated polynucleotide comprising a nucleic acid encoding a subunit polypeptide of the multimeric binding molecule of any one of claims 1 to 74, wherein the subunit polypeptide comprises (a) an IgA or IgM heavy chain comprising an IgA or IgM heavy chain constant region or a multimerizing variant or fragment thereof associated with an antibody heavy chain variable region (VH), (b) an antibody light chain comprising an antibody light chain constant region associated with an antibody light chain variable region (VL), or (c) a modified J-chain comprising two or more of (i) a J-chain or functional fragment or variant thereof (“J”), (ii) an interleukin-15 (IL-15) protein or receptor-binding fragment or variant thereof (“I”), or (iii) an interleukin-15 receptor-α (IL-15Rα) fragment comprising the sushi domain or a variant thereof capable of associating with I (“R”), wherein J and at least one of I and R are associated as a fusion protein, and wherein I and R can associate to function as an immune stimulatory complex, or (d) any combination thereof.

76. The polynucleotide of claim 75, wherein the subunit polypeptide comprises an IgM heavy chain comprising an IgM constant region or multimerizing fragment or variant thereof.

77. The polynucleotide of claim 76, wherein the IgM constant region or fragment or variant thereof comprises the amino acid sequence SEQ ID NO: 51 or SEQ ID NO: 52.

78. The polynucleotide of claim 75, wherein the subunit polypeptide comprises the antibody light chain.

79. The polynucleotide of claim 75, wherein the subunit polypeptide comprises the modified J-chain.

80. The polynucleotide of claim 79, wherein the subunit comprises the amino acid sequence SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 15, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, or SEQ ID NO: 32.

81. The polynucleotide of any one of claims 75 to 80, which comprises two, three, or more nucleic acid sequences encoding two, three or more of the subunit polypeptides.

82. An expression vector comprising the polynucleotide of any one of claims 75 to 81.

83. A host cell comprising the polynucleotide of any of claims 75 to 81 or the expression vector of claim 82.

84. A method for producing the multimeric binding molecule of any one of claims 1 to 74, comprising culturing the host cell of claim 83, and recovering the multimeric binding molecule.

85. A method for treating cancer, comprising administering to a subject in need of treatment the multimeric binding molecule of any one of claims 1 to 74.
